SEQUENCE LISTING (114 sequences)

SEQ ID NO: 1 - RNA, 22 nt, Artificial Sequence
  oligonucleotide
gucauacacggcucuccucucu

SEQ ID NO: 2 - RNA, 7 nt, Artificial Sequence
  oligonucleotide
uguauga

SEQ ID NO: 3 - RNA, 8 nt, Artificial Sequence
  oligonucleotide
guguauga

SEQ ID NO: 4 - RNA, 9 nt, Artificial Sequence
  oligonucleotide
cguguauga

SEQ ID NO: 5 - RNA, 10 nt, Artificial Sequence
  oligonucleotide
ccguguauga

SEQ ID NO: 6 - RNA, 11 nt, Artificial Sequence
  oligonucleotide
gccguguauga

SEQ ID NO: 7 - RNA, 12 nt, Artificial Sequence
  oligonucleotide
agccguguauga

SEQ ID NO: 8 - RNA, 13 nt, Artificial Sequence
  oligonucleotide
gagccguguauga

SEQ ID NO: 9 - RNA, 14 nt, Artificial Sequence
  oligonucleotide
agagccguguauga

SEQ ID NO: 10 - RNA, 15 nt, Artificial Sequence
  oligonucleotide
gagagccguguauga

SEQ ID NO: 11 - RNA, 16 nt, Artificial Sequence
  oligonucleotide
ggagagccguguauga

SEQ ID NO: 12 - RNA, 17 nt, Artificial Sequence
  oligonucleotide
aggagagccguguauga

SEQ ID NO: 13 - RNA, 18 nt, Artificial Sequence
  oligonucleotide
gaggagagccguguauga

SEQ ID NO: 14 - RNA, 19 nt, Artificial Sequence
  oligonucleotide
agaggagagccguguauga

SEQ ID NO: 15 - RNA, 20 nt, Artificial Sequence
  oligonucleotide
gagaggagagccguguauga

SEQ ID NO: 16 - RNA, 8 nt, Artificial Sequence
  oligonucleotide
uguaugac

SEQ ID NO: 17 - RNA, 9 nt, Artificial Sequence
  oligonucleotide
guguaugac

SEQ ID NO: 18 - RNA, 10 nt, Artificial Sequence
  oligonucleotide
cguguaugac

SEQ ID NO: 19 - RNA, 11 nt, Artificial Sequence
  oligonucleotide
ccguguaugac

SEQ ID NO: 20 - RNA, 12 nt, Artificial Sequence
  oligonucleotide
gccguguaugac

SEQ ID NO: 21 - RNA, 13 nt, Artificial Sequence
  oligonucleotide
agccguguaugac

SEQ ID NO: 22 - RNA, 14 nt, Artificial Sequence
  oligonucleotide
gagccguguaugac

SEQ ID NO: 23 - RNA, 15 nt, Artificial Sequence
  oligonucleotide
agagccguguaugac

SEQ ID NO: 24 - RNA, 16 nt, Artificial Sequence
  oligonucleotide
gagagccguguaugac

SEQ ID NO: 25 - RNA, 17 nt, Artificial Sequence
  oligonucleotide
ggagagccguguaugac

SEQ ID NO: 26 - RNA, 18 nt, Artificial Sequence
  oligonucleotide
aggagagccguguaugac

SEQ ID NO: 27 - RNA, 19 nt, Artificial Sequence
  oligonucleotide
gaggagagccguguaugac

SEQ ID NO: 28 - RNA, 20 nt, Artificial Sequence
  oligonucleotide
agaggagagccguguaugac

SEQ ID NO: 29 - RNA, 21 nt, Artificial Sequence
  oligonucleotide
gagaggagagccguguaugac

SEQ ID NO: 30 - RNA, 22 nt, Artificial Sequence
  oligonucleotide
agagaggagagccguguaugac

SEQ ID NO: 31 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 32 - protein, 561 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 33 - RNA, 22 nt, Homo sapiens
agaggcuggccgugaugaauuc

SEQ ID NO: 34 - RNA, 22 nt, Artificial Sequence
  miR-485-3p
agucauacacggcucuccucuc

SEQ ID NO: 35 - RNA, 22 nt, Artificial Sequence
  miR-485-5p
agaggcuggccgugaugaauuc

SEQ ID NO: 36 - protein, 472 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 37 - protein, 288 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRETCVHFTSSFSVCKS

SEQ ID NO: 38 - protein, 433 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 39 - protein, 412 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 40 - protein, 798 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 41 - protein, 271 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNLFL

SEQ ID NO: 42 - protein, 803 aa, Homo sapiens
MDETSPRLEEDWKKVLQREAGWQCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 43 - protein, 786 aa, Homo sapiens
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 44 - protein, 289 aa, Homo sapiens
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGVKTNLISK

SEQ ID NO: 45 - protein, 276 aa, Homo sapiens
MDETSPRLEEDWKKVLQREAGWQCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNLFL

SEQ ID NO: 46 - protein, 138 aa, Homo sapiens
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLVRTLPTV

SEQ ID NO: 47 - protein, 301 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGVKTNLISK

SEQ ID NO: 48 - protein, 671 aa, Homo sapiens
MPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 49 - RNA, 7 nt, Artificial Sequence
  oligonucleotide
ucauaca

SEQ ID NO: 50 - RNA, 7 nt, Artificial Sequence
  oligonucleotide
gaggcug

SEQ ID NO: 51 - DNA, 18 bp, Artificial Sequence
  oligonucleotide
cgaggtcgacttcctaga

SEQ ID NO: 52 - DNA, 23 bp, Artificial Sequence
  oligonucleotide
catacacggctctcctctctaaa

SEQ ID NO: 53 - DNA, 20 bp, Artificial Sequence
  oligonucleotide
tcctgtggcatccatgaaac

SEQ ID NO: 54 - DNA, 20 bp, Artificial Sequence
  oligonucleotide
caatgcctgggtacatggtg

SEQ ID NO: 55 - DNA, 18 bp, Artificial Sequence
  oligonucleotide
ccaagtggaggagcagct

SEQ ID NO: 56 - DNA, 20 bp, Artificial Sequence
  oligonucleotide
gacaaggtacaacccatcgg

SEQ ID NO: 57 - DNA, 22 bp, Artificial Sequence
  oligonucleotide
ttcgacacatgggataacgagg

SEQ ID NO: 58 - DNA, 21 bp, Artificial Sequence
  oligonucleotide
tttttgctgtgagtcccggag

SEQ ID NO: 59 - DNA, 20 bp, Artificial Sequence
  oligonucleotide
cagcctagcagcacgtaaat

SEQ ID NO: 60 - DNA, 19 bp, Artificial Sequence
  oligonucleotide
gaatcgagcaccagttacg

SEQ ID NO: 61 - DNA, 22 bp, Artificial Sequence
  oligonucleotide
ccttccctgaaggttcctcctt

SEQ ID NO: 62 - DNA, 7 bp, artificial Sequence
  oligonucleotide
tgtatga

SEQ ID NO: 63 - DNA, 8 bp, artificial Sequence
  oligonucleotide
gtgtatga

SEQ ID NO: 64 - DNA, 9 bp, artificial Sequence
  oligonucleotide
cgtgtatga

SEQ ID NO: 65 - DNA, 10 bp, artificial Sequence
  oligonucleotide
ccgtgtatga

SEQ ID NO: 66 - DNA, 11 bp, artificial Sequence
  oligonucleotide
gccgtgtatga

SEQ ID NO: 67 - DNA, 12 bp, artificial Sequence
  oligonucleotide
agccgtgtatga

SEQ ID NO: 68 - DNA, 13 bp, artificial Sequence
  oligonucleotide
gagccgtgtatga

SEQ ID NO: 69 - DNA, 14 bp, artificial Sequence
  oligonucleotide
agagccgtgtatga

SEQ ID NO: 70 - DNA, 15 bp, artificial Sequence
  oligonucleotide
gagagccgtgtatga

SEQ ID NO: 71 - DNA, 16 bp, artificial Sequence
  oligonucleotide
ggagagccgtgtatga

SEQ ID NO: 72 - DNA, 17 bp, artificial Sequence
  oligonucleotide
aggagagccgtgtatga

SEQ ID NO: 73 - DNA, 18 bp, artificial Sequence
  oligonucleotide
gaggagagccgtgtatga

SEQ ID NO: 74 - DNA, 19 bp, artificial Sequence
  oligonucleotide
agaggagagccgtgtatga

SEQ ID NO: 75 - DNA, 20 bp, artificial Sequence
  oligonucleotide
gagaggagagccgtgtatga

SEQ ID NO: 76 - DNA, 8 bp, artificial Sequence
  oligonucleotide
tgtatgac

SEQ ID NO: 77 - DNA, 9 bp, artificial Sequence
  oligonucleotide
gtgtatgac

SEQ ID NO: 78 - DNA, 10 bp, artificial Sequence
  oligonucleotide
cgtgtatgac

SEQ ID NO: 79 - DNA, 11 bp, artificial Sequence
  oligonucleotide
ccgtgtatgac

SEQ ID NO: 80 - DNA, 12 bp, artificial Sequence
  oligonucleotide
gccgtgtatgac

SEQ ID NO: 81 - DNA, 13 bp, artificial Sequence
  oligonucleotide
agccgtgtatgac

SEQ ID NO: 82 - DNA, 14 bp, artificial Sequence
  oligonucleotide
gagccgtgtatgac

SEQ ID NO: 83 - DNA, 15 bp, artificial Sequence
  oligonucleotide
agagccgtgtatgac

SEQ ID NO: 84 - DNA, 16 bp, artificial Sequence
  oligonucleotide
gagagccgtgtatgac

SEQ ID NO: 85 - DNA, 17 bp, artificial Sequence
  oligonucleotide
ggagagccgtgtatgac

SEQ ID NO: 86 - DNA, 18 bp, artificial Sequence
  oligonucleotide
aggagagccgtgtatgac

SEQ ID NO: 87 - DNA, 19 bp, artificial Sequence
  oligonucleotide
gaggagagccgtgtatgac

SEQ ID NO: 88 - DNA, 20 bp, artificial Sequence
  oligonucleotide
agaggagagccgtgtatgac

SEQ ID NO: 89 - DNA, 21 bp, artificial Sequence
  oligonucleotide
gagaggagagccgtgtatgac

SEQ ID NO: 90 - DNA, 22 bp, artificial Sequence
  oligonucleotide
agagaggagagccgtgtatgac

SEQ ID NO: 91 - DNA, 22 bp, Artificial Sequence
  Oligonucleotide
ucucuccucucggcacauacug

SEQ ID NO: 92 - DNA, 22 bp, Artificial Sequence
  Oligonucleotide
uuuaaagguucauuuguaugau

SEQ ID NO: 93 - DNA, 22 bp, Artificial Sequence
  Oligonucleotide
uuuaaagguucauuagatagtu

SEQ ID NO: 94 - DNA, 22 bp, artificial sequence
  miR485-3p_FW1 primer
gtcatacacggctctcctctct

SEQ ID NO: 95 - DNA, 20 bp, Artificial Sequence
  miR485-3p_FW2 primer
tcatacacggctctcctctc

SEQ ID NO: 96 - DNA, 19 bp, Artificial Sequence
  miR485-3p_FW3 primer
catacacggctctcctctc

SEQ ID NO: 97 - DNA, 21 bp, Artificial Sequence
  miR485-3p_FW4 primer
catacacggctctcctctcta

SEQ ID NO: 98 - DNA, 19 bp, Artificial Sequence
  miR485-3p_FW5 primer
catacacggctctcgtctc

SEQ ID NO: 99 - DNA, 22 bp, Artificial Sequence
  miR485-3p_FW6 primer
catacacggctctcgtctctaa

SEQ ID NO: 100 - DNA, 24 bp, Artificial Sequence
  miR485-3p_FW7 primer
gtcatacacggctctcctctctaa

SEQ ID NO: 101 - DNA, 19 bp, Artificial Sequence
  miR485-3p_FW8 primer
gtcatacacggctctcctc

SEQ ID NO: 102 - DNA, 21 bp, Artificial Sequence
  miR485-3p_FW10 primer
gtcatacacggctctcctctg

SEQ ID NO: 103 - DNA, 21 bp, Artificial Sequence
  miR485-3p_FW11 primer
tcatacacggctctcctctct

SEQ ID NO: 104 - DNA, 18 bp, Artificial Sequence
  miR485-3p_FW12 primer
tcatacacggctctcctc

SEQ ID NO: 105 - DNA, 23 bp, Artificial Sequence
  miR485-3p_FW13 primer
tcatacacggctctcctctctaa

SEQ ID NO: 106 - DNA, 22 bp, Artificial Sequence
  miR485-3p_FW14 primer
catacacggctctcctctctaa

SEQ ID NO: 107 - DNA, 21 bp, Artificial Sequence
  miR485-3p_FW15 primer
atacacggctctcctctctaa

SEQ ID NO: 108 - DNA, 18 bp, Artificial Sequence
  Fluorescent probe
cgaggtcgacttcctaga

SEQ ID NO: 109 - DNA, 19 bp, Artificial Sequence
  E2-Forward primer
gcggacatggaggacgtgt

SEQ ID NO: 110 - DNA, 19 bp, Artificial Sequence
  E2-Reverse primer
cctggtacactgccaggca

SEQ ID NO: 111 - DNA, 18 bp, Artificial Sequence
  E3-Forward primer
cggacatggaggacgtgt

SEQ ID NO: 112 - DNA, 18 bp, Artificial Sequence
  E3/E4-Reverse primer
ctggtacactgccaggcg

SEQ ID NO: 113 - DNA, 18 bp, Artificial Sequence
  E4-Forward primer
cggacatggaggacgtgc

SEQ ID NO: 114 - DNA, 20 bp, Artificial Sequence
  ApoE flouorescent probe
cagctcctcggtgctctggc

(19) United States
(12) Patent Application Publication
(10) Pub. No.: US 2023/0167502 A1
(43) Pub. Date: Jun. 1, 2023

(54) DIAGNOSTIC METHODS USING MIR-485-3P EXPRESSION

(71) Applicant: BIORCHESTRA CO., LTD., Daejeon (KR)

(72) Inventors: Jin-Hyeob RYU, Daejeon (KR); Dae Hoon KIM, Daejeon (KR); Jae Woong MIN, Daejeon (KR); Byung Gyu PARK, Daejeon (KR); Hyun Su MIN, Daejeon (KR); Yu Na LIM, Daejeon (KR)

(73) Assignee: BIORCHESTRA CO., LTD., Daejeon (KR)

(21) Appl. No.: 17/997,000

(22) PCT Filed: Apr. 23, 2021
(86) PCT No.: PCT/IB2021/053353

§ 371 Date: Oct. 24, 2022

Related U.S. Application Data
(60) Provisional application No. 63/064,305, filed on Aug. 11, 2020.
(60) Provisional application No. 63/047,206, filed on Jul. 1, 2020.
(60) Provisional application No. 63/014,633, filed on Apr. 23, 2020.

(51) Int. Cl. C12Q 1/6883; C12Q 1/686

(52) U.S. Cl. CPC C12Q 1/6883; C12Q 1/686; C12Q 2600/158; C12Q 2600/178

(57) ABSTRACT

The present disclosure relates to the use of miR-485-3p expression to identify a subject that is afflicted with a cognitive disorder. In some aspects, the methods disclosed herein further comprises administering a miR-485-3p inhibitor to the subject, wherein the miR-485-3p inhibitor is capable of treating the cognitive disorder.

### CROSS-REFERENCE TO RELATED APPLICATIONS

This PCT application claims the priority benefit of U.S. Provisional Application Nos. 63/014,633, filed Apr. 23, 2020; 63/047,206, filed Jul. 1, 2020; and 63/064,305, filed Aug. 11, 2020; each of which is incorporated herein by reference in its entirety.

### REFERENCE TO SEQUENCE LISTING SUBMITTED ELECTRONICALLY VIA EFS-WEB

The content of the electronically submitted sequence listing in ASCII text file (Name: 4366_025PC03_Seglisting_ST25.txt; Size: 81,709 bytes; and Date of Creation: Apr. 22, 2021) filed with the application is herein incorporated by reference in its entirety.

### FIELD OF THE DISCLOSURE

The present disclosure provides methods of identifying a subject afflicted with a cognitive disorder (e.g., Alzheimer's disease) comprising measuring the miR-485-3p level of a subject (e.g., in a biological sample derived from the subject). The present disclosure further provides methods for treating a cognitive disorder in a subject identified as having an increase in miR-485-3p level.

### BACKGROUND OF THE DISCLOSURE

Cognitive disorders, such as Alzheimer's disease (AD), are common and growing cause of mortality and morbidity worldwide. It is estimated that by 2050, more than 100 million people worldwide will be affected by AD. Gaugler et al., Alzheimer's Dement 12(4): 459-509 (2016); Pan et al., Sci Adv 5(2) (2019). The costs of AD are estimated at more than 800 billion USD globally. To date, researchers have largely been unsuccessful in developing compounds (e.g., antibodies) that can effectively inhibit the production and/or aggregation of amyloid-β, and/or promote their clearance in a human subject.

Accordingly, there is still no known cure for AD and similar cognitive disorders. Available treatment options are generally limited to alleviating the various symptoms, as opposed to addressing the underlying causes of the disorders. In addition, no effective early diagnostic system is available, and therefore, any treatment options to help alleviate some of the symptoms associated with cognitive disorders are not made available until long after the onset of the disorder. And, the available methods of diagnosing cognitive disorders are often subjective (e.g., questionnaires), potentially harmful (e.g., use of radioactive isotopes for nuclear brain imaging), and/or expensive. Therefore, new and more effective approaches to treating and/or diagnosing cognitive disorders are highly desirable.

### BRIEF SUMMARY OF THE DISCLOSURE

Provided herein is a method of identifying a human subject afflicted with a cognitive disorder comprising measuring a level of miR-485-3p in a biological sample derived from an epithelial cell or serum of the subject. In some aspects, the biological sample is an extracellular vesicle.

Also provided herein is a method of identifying a subject afflicted with a cognitive disorder comprising measuring a level of miR-485-3p in a biological sample obtained from the subject, wherein the biological sample comprises an extracellular vesicle.

In some aspects, the extracellular vesicle is obtained from an epithelial cell of the subject. In some aspects, the epithelial cell is an oral mucosal epithelial cell. In some aspects, the extracellular vesicle is obtained from serum of the subject. In certain aspects, the extracellular vesicle comprises a microvesicle. In some aspects, the extracellular vesicle comprises an exosome.

In some aspects, the level of miR-485-3p is increased in the subject compared to a reference level (e.g., a miR-485-3p expression level in a subject without a cognitive disorder or a miR-485-3p level prior to having a cognitive disorder in the subject). In certain aspects, the level of miR-485-3p is increased in the subject by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more, compared to the reference level.

In some aspects, the methods provided above further comprises administering a therapy to treat the cognitive disorder.

Provided herein is a method of treating a cognitive disorder in a human subject in need thereof comprising administering a therapy to treat the cognitive disorder to a human subject identified as having an increased level of miR-485-3p in a biological sample derived from an epithelial cell or serum of the subject, compared to a reference level (e.g., a miR-485-3p expression level in a subject without a cognitive disorder or a miR-485-3p level prior to having a cognitive disorder in the subject).

In some aspects, the biological sample is an extracellular vesicle. In certain aspects, the extracellular vesicle is obtained from an epithelial cell of the subject. In some aspects, the epithelial cell is an oral mucosal epithelial cell. In some aspects, the extracellular vesicle is obtained from serum of the subject. In some aspects, the extracellular vesicle comprises a microvesicle. In some aspects, the extracellular vesicle comprises an exosome.

In some aspects, the level of miR-485-3p in the biological sample is measured using a polymerase chain reaction (PCR) assay. In certain aspects, the PCR assay comprises a real time PCR. In some aspects, the measuring comprises determining a cycle threshold (Ct) value of miR-485-3p.

In some aspects, the methods described above further comprises measuring an additional factor regarding the subject, wherein the additional factor is selected from age, gender, education year (EDU), apolipoprotein E (APOE) genotype, Mini Mental State Examination (MMSE) score, or any combination thereof.

In some aspects, the additional factors are gender and education year. In certain aspects, the additional factor is gender. In some aspects, the gender comprises male or female, and wherein male is associated with a value of 1 and female is associated with a value of 2. In some aspects, the APOE genotype comprises (i) E2/E3, which is associated with a value of 1, (ii) E3/E3, which is associated with a value of 1, (iii) E2/E4, which is associated with a value of 2, (iv) E3/E4, which is associated with a value of 2, or (v) E4/E4. In some aspects, the education year comprises a value between 0 and 16.

In some aspects, the method which comprises measuring an additional factor regarding the subject, further comprises calculating a diagnostic score of the subject using the following formula: (Naïve Ct×(Gender×V1_{Gender}+V2_{Gender}))×(Education year×V1_{EDU}+V2_{EDU}), wherein V1 and V2 are regression coefficient values associated with the specific additional factor. In some aspects, the method further comprises calculating a diagnostic score of the subject using the following formula: (Naïve CT×(Age×V1_{Age}+V2_{Age}))×(Gender×V1_{Gender}+V2_{Gender})×(APOE×V1_{APOE}+V2_{APOE})×(MMSE×V1_{MMSE}+V2_{MMSE})×(Education year×V1_{EDU}+V2_{EDU}), wherein V1 and V2 are regression coefficient values associated with the specific additional factor. In some aspects, the method further comprises calculating a diagnostic score of the subject using the following formula: (Naïve CT×(Gender×V1_{Gender}+V2_{Gender})), wherein V1 and V2 are regression coefficient values associated with the specific additional factor.

In some aspects, measuring the level of miR-485-3p in the biological sample of a subject comprises using one or more miR-485-3p primers to amplify the miR-485-3p present in the biological sample.

Also provided herein is a method of determining a level of miR-485-3p in a subject afflicted with a cognitive disorder, comprising detecting whether the level of miR-485-3p in a biological sample obtained from the subject is increased compared to a reference level (e.g., a miR-485-3p expression level in a subject without a cognitive disorder or a miR-485-3p level prior to having a cognitive disorder in the subject) by amplifying the miR-485-3p present in the biological sample with one or more miR-485-3p primers.

In some aspects, the level of miR-485-3p is increased in the subject by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more, compared to the reference level.

In some aspects, the biological sample comprises a tissue, cell, blood, serum, saliva, or combinations thereof. In certain aspects, the biological sample comprises an extracellular vesicle. In some aspects, the extracellular vesicle is obtained from an epithelial cell of the subject. In some aspects, the epithelial cell is an oral mucosal epithelial cell. In some aspects, the extracellular vesicle is obtained from serum of the subject. In certain aspects, the extracellular vesicle comprises a microvesicle. In some aspects, the extracellular vesicle comprises an exosome.

In some aspects, the miR-485-3p primers comprise miR-485-3p_FW1 (GTCATACACGGCTCTCCTCTCT) (SEQ ID NO: 94), miR-485-3p_FW2 (TCATACACGGCTCTCCTCTC) (SEQ ID NO: 95), miR-485-3p_FW3 (CATACACGGCTCTCCTCTC) (SEQ ID NO: 96), miR-485-3p_FW4 (CATACACGGCTCTCCTCTCTA) (SEQ ID NO: 97), miR-485-3p_FW5 (CATACACGGCTCTCGTCTC) (SEQ ID NO: 98), miR-485-3p_FW6 (CATACACGGCTCTCGTCTCTAA) (SEQ ID NO: 99), miR-485-3p_FW7 (GTCATACACGGCTCTCCTCTCTAA) (SEQ ID NO: 100), miR-485-3p_FW8 (GTCATACACGGCTCTCCTC) (SEQ ID NO: 101), miR-485-3p_FW9 (CATACACGGCTCTCCTCTCTAAA) (SEQ ID NO: 52), miR-485-3p_FW10 (GTCATACACGGCTCTCCTCTG) (SEQ ID NO: 102), miR-485-3p_FW11 (TCATACACGGCTCTCCTCTCT) (SEQ ID NO: 103), miR-485-3p_FW12 (TCATACACGGCTCTCCTC) (SEQ ID NO: 104), miR-485-3p_FW13 (TCATACACGGCTCTCCTCTCTAA) (SEQ ID NO: 105), miR-485-3p_FW14 (CATACACGGCTCTCCTCTCTAA) (SEQ ID NO: 106), miR-485-3p_FW15 (ATACACGGCTCTCCTCTCTAA) (SEQ ID NO: 107), or any combination thereof. In certain aspects, the miR-485-3p primers comprise miR-485-3p_FW7. In certain aspects, the miR-485-3p primers comprise miR-485-3p_FW2. In some aspects, the miR-485-3p primers comprise miR-485-3p_FW1. In some aspects, the miR-485-3p primers comprise miR-485-3p_FW9.

In some aspects, the method of determining a level of miR-485-3p in a subject afflicted with a cognitive disorder disclosed herein further comprises administering a therapy capable of treating the cognitive disorder.

In some aspects, a therapy that can be used in combination with the methods disclosed herein comprises a miR-485-3p inhibitor (also referred to herein as “miRNA inhibitor”).

In some aspects, the miR-485-3p inhibitor comprises a nucleotide sequence comprising 5′-UGUAUGA-3′ (SEQ ID NO: 2) and wherein the miR-485-3p inhibitor comprises about 6 to about 30 nucleotides in length. In some aspects, the miR-485-3p inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 5′ of the nucleotide sequence; and/or the miR-485-3p inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 3′ of the nucleotide sequence.

In some aspects, the miR-485-3p inhibitor comprises a nucleotide sequence selected from the group consisting of: 5′-UGUAUGA-3′ (SEQ ID NO: 2), 5′-GUGUAUGA-3′ (SEQ ID NO: 3), 5′-CGUGUAUGA-3′ (SEQ ID NO: 4), 5′-CCGUGUAUGA-3′ (SEQ ID NO: 5), 5′-GCCGUGUAUGA-3′ (SEQ ID NO: 6), 5′-AGCCGUGUAUGA-3′ (SEQ ID NO: 7), 5′-GAGCCGUGUAUGA-3′ (SEQ ID NO: 8), 5′-AGAGCCGUGUAUGA-3′ (SEQ ID NO: 9), 5′-GAGAGCCGUGUAUGA-3′ (SEQ ID NO: 10), 5′-GGAGAGCCGUGUAUGA-3′ (SEQ ID NO: 11), 5′-AGGAGAGCCGUGUAUGA-3′ (SEQ ID NO: 12), 5′-GAGGAGAGCCGUGUAUGA-3′ (SEQ ID NO: 13), 5′-AGAGGAGAGCCGUGUAUGA-3′ (SEQ ID NO: 14), 5′-GAGAGGAGAGCCGUGUAUGA-3′ (SEQ ID NO: 15); 5′-UGUAUGAC-3′ (SEQ ID NO: 16), 5′-GUGUAUGAC-3′ (SEQ ID NO: 17), 5′-CGUGUAUGAC-3′ (SEQ ID NO: 18), 5′-CCGUGUAUGAC-3′ (SEQ ID NO: 19), 5′-GCCGUGUAUGAC-3′ (SEQ ID NO: 20), 5′-AGCCGUGUAUGAC-3′ (SEQ ID NO: 21), 5′-GAGCCGUGUAUGAC-3′ (SEQ ID NO: 22), 5′-AGAGCCGUGUAUGAC-3′ (SEQ ID NO: 23), 5′-GAGAGCCGUGUAUGAC-3′ (SEQ ID NO: 24), 5′-GGAGAGCCGUGUAUGAC-3′ (SEQ ID NO: 25), 5′-AGGAGAGCCGUGUAUGAC-3′ (SEQ ID NO: 26), 5′-GAGGAGAGCCGUGUAUGAC-3′ (SEQ ID NO: 27), 5′-AGAGGAGAGCCGUGUAUGAC-3′ (SEQ ID NO: 28), 5′-GAGAGGAGAGCCGUGUAUGAC-3′ (SEQ ID NO: 29), and 5′-AGAGAGGAGAGCCGUGUAUGAC-3′ (SEQ ID NO: 30).

In some aspects, the miR-485-3p inhibitor has a sequence selected from the group consisting of: 5′-TGTATGA-3′ (SEQ ID NO: 62), 5′-GTGTATGA-3′ (SEQ ID NO: 63), 5′-CGTGTATGA-3′ (SEQ ID NO: 64), 5′-CCGTGTATGA-3′ (SEQ ID NO: 65), 5′-GCCGTGTATGA-3′ (SEQ ID NO: 66), 5′-AGCCGTGTATGA-3′ (SEQ ID NO: 67), 5′-GAGCCGTGTATGA-3′ (SEQ ID NO: 68), 5′-AGAGCCGTGTATGA-3′ (SEQ ID NO: 69), 5′-GAGAGCCGTGTATGA-3′ (SEQ ID NO: 70), 5′-GGAGAGCCGTGTATGA-3′ (SEQ ID NO: 71), 5′-AGGAGAGCCGTGTATGA-3′ (SEQ ID NO: 72), 5′-GAGGAGAGCCGTGTATGA-3′ (SEQ ID NO: 73), 5′-AGAGGAGAGCCGTGTATGA-3′ (SEQ ID NO: 74), 5′-GAGAGGAGAGCCGTGTATGA-3′ (SEQ ID NO: 75); 5′-TGTATGAC-3′ (SEQ ID NO: 76), 5′-GTGTATGAC-3′ (SEQ ID NO: 77), 5′-CGTGTATGAC-3′ (SEQ ID NO: 78), 5′-CCGTGTATGAC-3′ (SEQ ID NO: 79), 5′-GCCGTGTATGAC-3′ (SEQ ID NO: 80), 5′-AGCCGTGTATGAC-3′ (SEQ ID NO: 81), 5′-GAGCCGTGTATGAC-3′ (SEQ ID NO: 82), 5′-AGAGCCGTGTATGAC-3′ (SEQ ID NO: 83), 5′-GAGAGCCGTGTATGAC-3′ (SEQ ID NO: 84), 5′-GGAGAGCCGTGTATGAC-3′ (SEQ ID NO: 85), 5′-AGGAGAGCCGTGTATGAC-3′ (SEQ ID NO: 86), 5′-GAGGAGAGCCGTGTATGAC-3′ (SEQ ID NO: 87), 5′-AGAGGAGAGCCGTGTATGAC-3′ (SEQ ID NO: 88), 5′-GAGAGGAGAGCCGTGTATGAC-3′ (SEQ ID NO: 89), and 5′-AGAGAGGAGAGCCGTGTATGAC-3′ (SEQ ID NO: 90).

In some aspects, the sequence of miR-485-3p inhibitor is at least about 50%, at least about 55%, at least about 60%, at least about 65%, at least about 70%, at least about 75%, at least about 80%, at least about 85%, at least about 90%, or at least about 95% sequence identity to 5′-AGAGAGGAGAGCCGUGUAUGAC-3′ (SEQ ID NO: 30) or 5′-AGAGAGGAGAGCCGTGTATGAC-3′ (SEQ ID NO: 90). In certain aspects, the miRNA inhibitor has a sequence that has at least 90% similarity to 5′-AGAGAGGAGAGCCGUGUAUGAC-3′ (SEQ ID NO: 30) or 5′-AGAGAGGAGAGCCGTGTATGAC-3′ (SEQ ID NO: 90). In some aspects, the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90) with one substitution or two substitutions. In some aspects, the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90). In some aspects, the miRNA inhibitor comprises the nucleotide sequence 5′-AGAGAGGAGAGCCGUGUAUGAC-3′ (SEQ ID NO: 30).

In some aspects, the miR-485-3p inhibitor comprises at least one modified nucleotide. In some aspects, the at least one modified nucleotide comprises a locked nucleic acid (LNA), unlocked nucleic acid (UNA), arabino nucleic acid (ABA), bridged nucleic acid (BNA), peptide nucleic acid (PNA), or any combination thereof.

In some aspects, the miR-485-3p inhibitor comprises a backbone modification. In some aspects, the backbone modification comprises a phosphorodiamidate morpholino oligomer (PMO) and/or phosphorothioate (PS) modification.

In some aspects, the miR-485-3p inhibitor is delivered by a viral vector. In some aspects, the viral vector is an AAV, an adenovirus, a retrovirus, or a lentivirus. In certain aspects, the viral vector is an AAV that has a serotype of AAV2, AAV3, AAV4, AAV5, AAV6, AAV7, AAV8, AAV9, AAV10, or any combination thereof.

In some aspects, the miR-485-3p inhibitor is delivered with a delivery agent. In some aspects, the delivery agent comprises a micelle, exosome, lipid nanoparticle, extracellular vesicle, synthetic vesicle, lipidoid, liposome, lipoplex, polymeric compound, peptide, protein, cell, nanoparticle mimic, nanotube, conjugate, viral vector, or any combination thereof. In some aspects, the delivery agent comprises a cationic carrier unit comprising

[WP]-L1-[CC]-L2-[AM] (formula I)

or

[WP]-L1-[AM]-L2-[CC] (formula II)

wherein
WP is a water-soluble biopolymer moiety;
CC is a positively charged (i.e., cationic) carrier moiety;
AM is an adjuvant moiety; and,
L1 and L2 are independently optional linkers

In some aspects, the miRNA inhibitor and the cationic carrier unit are capable of associating with each other to form a micelle when mixed together. In certain aspects, the association is via a covalent bond. In some aspects, the association is via a non-covalent bond. In some aspects, the miRNA inhibitor interacts with the cationic carrier unit via an ionic bond. In some aspects, the cationic carrier unit is capable of protecting the miRNA inhibitor from enzymatic degradation.

In some aspects, the water-soluble polymer moiety comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof. In some aspects, the water-soluble polymer comprises polyethylene glycol (“PEG”), polyglycerol, or poly(propylene glycol) (“PPG”).

In some aspects, the water-soluble polymer moiety comprises:

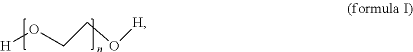

wherein n is 1-1000.

In some aspects, the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In certain aspects, the n is about 80 to about 90, about 90 to about 100, about 100 to about 110, about 110 to about 120, about 120 to about 130, about 140 to about 150, about 150 to about 160.

In some aspects, the water-soluble polymer moiety is linear, branched, or dendritic.

In some aspects, the cationic carrier moiety comprises one or more basic amino acids. In certain aspects, the cationic carrier moiety comprises at least about three, at least about four, at least about five, at least about six, at least about seven, at least about eight, at least about nine, at least about ten, at least about 11, at least about 12, at least about 13, at least about 14, at last about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, or at least about 50 basic amino acids.

In some aspects, the basic amino acid comprises arginine, lysine, histidine, or any combination thereof. In some aspects, the cationic carrier moiety comprises about 40 lysine monomers.

In some aspects, the adjuvant moiety is capable of modulating an immune response, an inflammatory response, and/or a tissue microenvironment. In some aspects, the adjuvant moiety comprises an imidazole derivative, an amino acid, a vitamin, or any combination thereof.

In some aspects, the adjuvant moiety comprises:

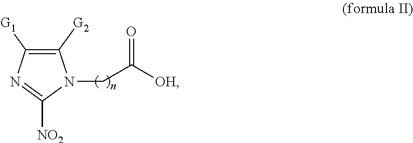

wherein each of G1 and G2 is H, an aromatic ring, or 1-10 alkyl, or G1 and G2 together form an aromatic ring, and wherein n is 1-10.

In some aspects, the adjuvant moiety comprises nitroimidazole. In certain aspects, the adjuvant moiety comprises metronidazole, tinidazole, nimorazole, dimetridazole, pretomanid, ornidazole, megazol, azanidazole, benznidazole, or any combination thereof.

In some aspects, the adjuvant moiety comprises an amino acid. In some aspects, the adjuvant moiety comprises

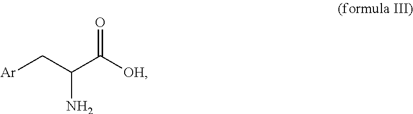

wherein Ar is

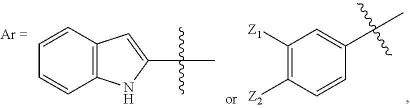

and
wherein each of Z1 and Z2 is H or OH.

In some aspects, the adjuvant moiety comprises a vitamin. In certain aspects, the vitamin comprises a cyclic ring or cyclic hetero atom ring and a carboxyl group or hydroxyl group.

In some aspects, the vitamin comprises:

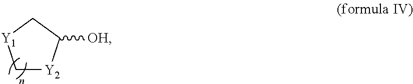

wherein each of Y1 and Y2 is C, N, O, or S, and wherein n is 1 or 2.

In some aspects, the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof. In certain aspects, the vitamin is vitamin B3.

In some aspects, the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3. In certain aspects, the adjuvant moiety comprises about 10 vitamin B3.

In some aspects, the delivery agent comprises about a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

In some aspects, the delivery agent is associated with the miR-485-3p inhibitor, thereby forming a micelle. In certain aspects, the association is a covalent bond, a non-covalent bond, or an ionic bond.

In some aspects, the cationic carrier unit and the miR-485-3p inhibitor in the micelle is mixed in a solution so that the ionic ratio of the positive charges of the cationic carrier unit and the negative charges of the miR-485-3p inhibitor is about 1:1. In some aspects, the cationic carrier unit is capable of protecting the miR-485-3p inhibitor from enzymatic degradation.

In some aspects, the cognitive disorder is associated with an increase in amyloid-beta accumulation within a region of the central nervous system (CNS) of the subject. In certain aspects, the region of the CNS comprises a brain. In some aspects, the cognitive disorder comprises an Alzheimer's Disease.

Also provided herein is a composition comprising a miR-485-3p primer which comprises miR485-3p_FW1 (GTCATACACGGCTCTCCTCTCT) (SEQ ID NO: 94), miR485-3p_FW2 (TCATACACGGCTCTCCTCTC) (SEQ ID NO: 95), miR485-3p_FW3 (CATACACGGCTCTCCTCTC) (SEQ ID NO: 96), miR485-3p_FW4 (CATACACGGCTCTCCTCTCTA) (SEQ ID NO: 97), miR485-3p_FW5 (CATACACGGCTCTCGTCTC) (SEQ ID NO: 98), miR485-3p_FW6 (CATACACGGCTCTCGTCTCTAA) (SEQ ID NO: 99), miR485-3p_FW7 (GTCATACACGGCTCTCCTCTCTAA) (SEQ ID NO: 100), miR-485-3p_FW8 (GTCATACACGGCTCTCCTC) (SEQ ID NO: 101), miR-485-3p_FW9 (CATACACGGCTCTCCTCTCTAAA) (SEQ ID NO: 52), miR-485-3p_FW10 (GTCATACACGGCTCTCCTCTG) (SEQ ID NO: 102), miR-485-3p_FW11 (TCATACACGGCTCTCCTCTCT) (SEQ ID NO: 103), miR-485-3p_FW12 (TCATACACGGCTCTCCTC) (SEQ ID NO: 104), miR-485-3p_FW13 (TCATACACGGCTCTCCTCTCTAA) (SEQ ID NO: 105), miR-485-3p_FW14 (CATACACGGCTCTCCTCTCTAA) (SEQ ID NO: 106), miR-485-3p_FW15 (ATACACGGCTCTCCTCTCTAA) (SEQ ID NO: 107), or any combination thereof.

In some aspects, the miR-485-3p primer comprises miR-485-3p_FW7. In some aspects, the miR-485-3p primer comprises miR-485-3p_FW2. In some aspects, the miR-485-3p primer comprises miR-485-3p_FW1. In some aspects, the miR-485-3p primer comprises miR-485-3p_FW9.

### BRIEF DESCRIPTION OF THE DRAWINGS/FIGURES

FIGS. 1A and 1B provide the diagnostic accuracy of using miR-485-3p expression (as measured with real-time PCR assay) in detecting amyloid-β accumulation in human clinical swab samples. FIG. 1A shows a comparison of the real-time PCR naïve cycle threshold (Ct) value of miR-485-3p in amyloid-β negative (i.e., without amyloid-β accumulation) (left) and amyloid-β positive (i.e., with amyloid-β accumulation) (right) samples. As explained in Example 1, the naïve Ct value is inversely related to expression value (i.e., higher miR-485-3p expression results in lower naïve Ct value). Amyloid-β accumulation was measured using amyloid PET scan. The horizontal dashed line represents the cutoff value between the amyloid PET negative and positive groups. FIG. 1B shows the specificity and sensitivity of using miR-485-3p expression in identifying clinical swab samples from patients with amyloid-β accumulation. Receiver Operating Characteristic (ROC) analysis was used to measure the following values: (i) area under the curve (AUC), (ii) sensitivity, (iii) specificity, and (iv) accuracy. The circular point identified by the arrow represents the specificity and sensitivity for the cutoff value shown in FIG. 1A. The numerical values for the AUC, accuracy, sensitivity, and specificity associated with the circular point is also provided in FIG. 1B.

FIGS. 2A and 2B demonstrate the effect that gender and education year has on the diagnostic accuracy of using miR-485-3p expression (as measured with real-time PCR assay) to detect amyloid-β accumulation in human clinical swab samples. FIG. 2A provides a comparison of scores for clinical swab samples from patients with (right) or without (left) amyloid-β accumulation. The scores were established using regression modeling based on a combination of miR-485-3p's naïve Ct value (see FIG. 1A), patient's gender, and patient's education level. See, e.g., Example 2 for the specific formula used to calculate the scores. The horizontal dashed line represents the cutoff value between the amyloid PET negative and positive groups. FIG. 2B shows the specificity and sensitivity of using a combination of miR-485-3p expression, gender, and education year in identifying clinical swab samples from patients with amyloid-β accumulation. Receiver Operating Characteristic (ROC) analysis was used to measure the following values: (i) area under the curve (AUC), (ii) sensitivity, (iii) specificity, and (iv) accuracy. The circular point identified by the arrow represents the specificity and sensitivity for the cutoff value shown in FIG. 2A. The numerical values for the AUC, accuracy, sensitivity, and specificity associated with the circular point is also provided in FIG. 2B.

FIGS. 3A and 3B demonstrate the effect that gender, age, mini mental stage examination (MMSE) score, APOE genotype, and education year have on the diagnostic accuracy of using miR-485-3p expression (as measured with real-time PCR assay) to detect amyloid-β accumulation in human clinical swab samples. FIG. 3A provides a comparison of scores for clinical swab samples from patients with (right) or without (left) amyloid-β accumulation. The scores were established using regression modeling based on a combination of miR-485-3p's naïve Ct value (see FIG. 1A), gender, age, MMSE score, APOE genotype, and education year. See, e.g., Example 2 for the specific formula used to calculate the scores. The horizontal dashed line represents the cutoff value between the amyloid PET negative and positive groups. FIG. 3B shows the specificity and sensitivity of using a combination of miR-485-3p expression (i.e., naïve Ct value), gender, age, MMSE score, APOE genotype, and education year in identifying clinical swab samples from patients with amyloid-β accumulation. Receiver Operating Characteristic (ROC) analysis was used to measure the following values: (i) area under the curve (AUC), (ii) sensitivity, (iii) specificity, and (iv) accuracy. The circular point identified by the arrow represents the specificity and sensitivity for the cutoff value shown in FIG. 3A. The numerical values for the AUC, accuracy, sensitivity, and specificity associated with the circular point is also provided in FIG. 3B.

FIG. 4 provides comparison of the results of regression modeling (i.e., % accuracy) based on miR-485-3p expression (i.e., naïve Ct value as determined using real-time PCR assay) in clinical swab samples in combination with one or more of the following clinical factors of the patients: age, gender, education year, APOE genotype, and MMSE score.

FIGS. 5A and 5B provide the diagnostic accuracy of using miR-485-3p expression (as measured with real-time PCR assay) in detecting amyloid-β accumulation in human clinical plasma samples. FIG. 5A shows a comparison of the real-time PCR naïve cycle threshold (Ct) value of miR-485-3p in amyloid-β negative (i.e., without amyloid-β accumulation) (left) and amyloid-β positive (i.e., with amyloid-β accumulation) (right) samples. As explained in Example 1, the naïve Ct value is inversely related to expression value (i.e., higher miR-485-3p expression results in lower naïve Ct value). Amyloid-β accumulation was measured using amyloid PET scan. The horizontal dashed line represents the cutoff value between the amyloid PET negative and positive groups. FIG. 5B shows the specificity and sensitivity of using miR-485-3p expression in identifying clinical plasma samples from patients with amyloid-β accumulation. Receiver Operating Characteristic (ROC) analysis was used to measure the following values: (i) area under the curve (AUC), (ii) sensitivity, (iii) specificity, and (iv) accuracy. The circular point identified by the arrow represents the specificity and sensitivity for the cutoff value shown in FIG. 5A. The numerical values for the AUC, accuracy, sensitivity, and specificity associated with the circular point is also provided in FIG. 5B.

FIGS. 6A and 6B demonstrate the effect that gender has on the diagnostic accuracy of using miR-485-3p expression (as measured with real-time PCR assay) to detect amyloid-β accumulation in human clinical plasma samples. FIG. 6A provides a comparison of scores for clinical plasma samples from patients with (right) or without (left) amyloid-β accumulation. The scores were established using regression modeling based on a combination of miR-485-3p's naïve Ct value (see FIG. 5A) and patient's gender. See, e.g., Example 4 for the specific formula used to calculate the scores. The horizontal dashed line represents the cutoff value between the amyloid PET negative and positive groups. FIG. 6B shows the specificity and sensitivity of using a combination of miR-485-3p expression (i.e., naïve Ct value) and gender in identifying clinical plasma samples from patients with amyloid-β accumulation. Receiver Operating Characteristic (ROC) analysis was used to measure the following values: (i) area under the curve (AUC), (ii) sensitivity, (iii) specificity, and (iv) accuracy. The circular point identified by the arrow represents the specificity and sensitivity for the cutoff value shown in FIG. 6A. The numerical values for the AUC, accuracy, sensitivity, and specificity associated with the circular point is also provided in FIG. 6B. FIG. 6C shows normalized expression of the data shown in FIGS. 6A and 6B. The normalized expression was calculated by a formula: 2^{-(Ct value of Real-Time PCR)}×10¹⁰. FIG. 6D shows the gender fitting score based on the data shown in FIGS. 6A and 6B. The score is a fitting value derived from regression modeling method using normalized expression value and patient-specific clinical information. In FIG. 6D, modeling was done with gender and normalized expression values.

FIG. 7 provides comparison of the results of regression modeling (i.e., % accuracy) based on miR-485-3p expression (i.e., naïve Ct value as determined using real-time PCR assay) in clinical plasma samples in combination with one or more of the following clinical factors of the patients: age, gender, education year, APOE genotype, and MMSE score.

FIGS. 8A, 8B, 8C, and 8D show the expression of miR-485-3p in human-derived oral epithelial cells treated with varying doses (i.e., 0.1, 0.5, or 1 μM) of amyloid-β monomer and oligomer, respectively. The expression level of miR-485-3p is shown normalized to the control (i.e., untreated cells) (see bar graphs shown on the left of each of the figures). In each of the figures, the graph provided on the right shows the positive correlation between miR-485-3p expression and the dose of amyloid-β treatment. The p values provided represent the p value of Pearson's correlation. “CC” represents the correlation coefficient of Pearson's correlation. In FIGS. 8A and 8B, the following primer was used in a real-time PCR assay to measure the miR-485-3p expression: 5′-GTCATACACGGCTCTCCTCTCT-3′ (referred to herein as “miR-485-3p_FW1”; SEQ ID NO: 94). In FIGS. 8C and 8D, the following primer was used: 5′-CATACACGGCTCTCCTCTCTAAA-3′ (referred to herein as “miR-485-3p_FW9”; SEQ ID NO: 52).

FIGS. 9A and 9B show the expression of miR-485-3p in the supernatant of human-derived oral epithelial cells treated with varying doses (i.e., 0.1 or 0.5 μM) of amyloid-β monomer and oligomer, respectively. The expression level of miR-485-3p is shown normalized to the control (i.e., untreated cells) (see bar graphs shown on the left of each of the figures). In each of FIGS. 9A and 9B, the graph provided on the right shows the positive correlation between miR-485-3p expression and the dose of amyloid-β treatment. The p-values provided represent the p value of Pearson's correlation. “CC” represents the correlation coefficient of Pearson's correlation.

FIG. 10A shows a comparison of scores for clinical plasma samples from 61 years old or higher patients with (right) (“Amyloid PET Positive”) or without (left) (“Amyloid PET Negative”) amyloid-β accumulation. p-value was measure by unpaired t test. ROC analysis based on target microRNA score of two group. Quantity value is the result of using the regression equation derived from the standard curve. FIG. 10B shows the specificity and sensitivity of using a combination of miR-485-3p expression (i.e., naïve Ct value) and gender in identifying clinical plasma samples from patients with amyloid-β accumulation. Receiver Operating Characteristic (ROC) analysis was used to measure the following values: (i) area under the curve (AUC), (ii) sensitivity, (iii) specificity, and (iv) accuracy. The circular point identified by the arrow represents the specificity and sensitivity for the cutoff value shown in FIG. 10A.

FIGS. 11A and 11B show miR-485-3p expression by Alzheimer's diagnosis. Of the patients diagnosed with Alzheimer's, the analysis was performed only on patients over 61 years old. NC (n=10) refers to a patient with a diagnosis of Alzheimer's diagnosis of “NC”, PET negative, and MMSE 25 or higher. MCI (n=4) refers to a patient with a diagnosis of Alzheimer's diagnosis of “MCI”, PET positive, and less than MMSE 25. AD (n=4) refers to a patient with a diagnosis of Alzheimer's diagnosis of “AD”, PET positive, and less than MMSE 25. P refers to p-value of Student's t Test result.

FIGS. 12A, 12B, and 12C show the identification of miR-485-3p as a candidate marker for the diagnosis of amyloid-β accumulation in human subjects. FIG. 12A provides a comparison of the expression of different miRNAs in AD patients and normal control subjects (i.e., subjects without AD). The miRNA expression is shown as fold change between the AD patients and normal control subjects (x-axis). The y-axis provides the p-value on a minus log 10 scale. Each circle represents an individual miRNA. The horizontal dotted line indicates a p-value of 0.05. The vertical dotted lines indicate ±1-fold change. FIG. 12B provides a comparison of miR-485-3p expression in both plasma and human oral-derived cell free exosomes (HOCFE) from individuals with amyloid-β accumulation (i.e., AD patients; “(1)”) and without amyloid-β accumulation (i.e., subjects without AD; “(2)”). miR-485-3p expression was calculated as 2^{-cycle threshold}×10¹³. “Q” refers to the log 10 (p-value). FIG. 12C provides a comparison of the specificity (y-axis) and sensitivity (x-axis) of the results provided in FIG. 12B. “AUC” refers to the area under the curve by ROC analysis.

FIGS. 13A, 13B, and 13C provides a comparison of AUCs for the different diagnostic methods disclosed in the present disclosure. In FIG. 13A, the AUC was generated using algorithms involving only clinical information. In FIG. 13B, the AUC was generated using algorithms involving both clinical information and the cycle threshold (Ct) value for miR-485-3p expression. In FIG. 13C, the AUCs was generated using algorithms involving the combination of clinical information and quantified miR-485-3p expression. Exemplary method of quantifying miR-485-3p expression is provided in Example 1 (see “Quantification of microRNA”). By comparing the AUCs, the algorithms involving the different diagnostic parameters were ranked as either 1^{st}, 2^{nd} or 3^{rd}. In each of the figures, the y-axis provides the number of algorithms that ranked 1^{st}, 2^{nd} or 3^{rd} for each of the different diagnostic parameters.

FIGS. 14A, 14B, and 14C show the relationship between miR-485-3p expression (measured in HOCFE) and patient's age. In FIG. 14A, patient samples were divided based on amyloid-O accumulation (i.e., amyloid PET positive or amyloid PET negative), then the expression of miR-485-3p is provided as a function of patient's age. Regression lines for both the amyloid PET positive and amyloid PET negative patients are provided. Regression line for the total patient population is also provided. Each of the circles represents an individual patient. FIG. 14B provides a comparison of the accuracy and AUC values for patients from each age group. As shown, the age group ranged from 53 years old to 86 years old. The red dotted lines represent the age group in which both the accuracy and AUC values were significantly high. The bar graph shown at the top shows the number of amyloid PET negative (light gray portion of the bar) and amyloid PET positive (dark gray portion of the bar) patients in each of the age groups. The box plot shown in FIG. 14C provides a comparison of miR-485-3p expression in amyloid PET negative and amyloid PET positive patients that are 61-73 years in age. The graph to the right provides the specificity and sensitivity values. AUC was measured by ROC analysis.

FIGS. 15A and 15B show exemplary methods that are useful in diagnosing amyloid-β accumulation based on miR-485-3p expression. FIG. 15A provides western blot analysis confirming the effectiveness of two exemplary methods used for RNA preparation from swab samples: (1) from cell pellets (“swab's pellet”); and (i) from human oral-derived cell free exosome (“swab's sup exosome”). For preparation from the cell pellets, calnexin was used as a marker for endoplasmic reticulum (ER). For preparation from human oral-derived cell free exosome, CD81 was used as marker. FIG. 15B provides a schematic of the different steps involved in using miR-485-3p expression in human oral-derived cell free exosomes (HOCFE) to diagnose amyloid-β accumulation. Briefly, HOCFE was collected using the swab method. Then, the miR-485-3p expression was quantified using a standard material and PCR analysis. Next, the miR-485-3p expression level was analyzed in combination with one or more of the patient's clinical information provided herein (e.g., age, gender, education year, MMSE score, APOE genotype, and CDR value). The analysis was confirmed by cross-validation methods.

FIGS. 16A and 16B show the effect of patient's clinical information on the accuracy of using miR-485-3p expression to diagnose amyloid-β accumulation in human subjects. FIG. 16A provides a comparison of AUC (area under the curve) value when patient's clinical information are considered alone or in combination with the miR-485-3p expression. “CFO” refers to clinical information only. “Ct value” refers to results using real-time PCR analysis that have not been quantified using standard materials (i.e., naïve cycle threshold values). “Quantity” refers to results that have been quantified using a standard material. “AUC” refers to the area under curve resulting from a comparison of the specificity and sensitivity values. AUC was measured by ROC analysis. FIG. 16B show the effect of patient's age on the predictive power of using miR-485-3p expression on diagnosing amyloid-β accumulation. The different age groups shown include: (i) 60 years old or less (“˜60”); (ii) 61-70 years old (“61˜70”); (iii) 71-80 years old (“71˜80”); and (iv) 81 years old or greater (“81˜”). The bar graphs at the top provide the accuracy data for the different age groups. Accuracy can be determined as follows: (total number of patients−number of false positives−number of false negatives)/(total number of patients). The bar graphs at the bottom provide a comparison of the miR-485-3p expression (quantity) in patients without amyloid-β accumulation (“1”; i.e., amyloid PET negative) and with amyloid-β accumulation (“2”; i.e., amyloid PET positive) from each of the age groups. The Q values provided refer to the minus log 10 of the p-value as measured using student's t-test. The “Count” along the x-axis refers to the number of samples from amyloid PET negative or amyloid PET positive patients from each age group.

FIGS. 17A, 17B, 17C, and 17D show the ability of miR-485-3p expression to accurately diagnose amyloid-β accumulation in patients within specific age groups. In FIGS. 17A and 17B, the age group ranged from 60 years old to about 90 years in age. In FIGS. 17C and 17D, the age group ranged from about 50 years old to about 85 years old. In FIGS. 17A and 17C, both the accuracy and AUC values are provided for patients as a function of age. The age group in which both the accuracy and AUC values are at significantly high levels are noted. The bar graph shown at the top shows the number of amyloid PET negative (light gray portion of the bar) and amyloid PET positive (dark gray portion of the bar) patients in each of the age groups. In FIGS. 17B and 17D provide comparison of miR-485-3p expression in amyloid PET negative and amyloid PET positive patients that are less than 73 years old (FIG. 17B) or above 68 years in age (FIG. 17D). The graph to the right provides the specificity and sensitivity values. AUC was measured by ROC analysis.

FIGS. 18A, 18B, 18C, 18D, 18E, and 18F show the number of significant model (left graph), AUC (middle graph), and error rate (right graph) for the following clinical information: age, education, MMSE score, gender, APOE score, and CDR score, respectively. The results are based on samples from patients who are 61 years old or older. As described further in Example 8, to construct the algorithms described in the present disclosure, the AUC and error rates were determined using regression modeling, in which simulation was repeated 100 times using random sampling up to 11^{th} dimension (i.e., also referred to in the art as order, degree, or polynomial). The results shown in the figures were used to determine the best regression dimension (i.e., white bar shown in the figures) for constructing the algorithms. The number of significant model value shown in FIG. 18A refer to the number of simulations (or tests) that were statistically significant (i.e., p value<0.05).

FIGS. 19A, 19B, 19C, 19D, 19E, and 19F show the same results provided in FIGS. 18A, 18B, 18C, 18D, 18E, and 18F except that the results are based on samples from all patients (i.e., not restricted to any age group).

FIGS. 20A, 20B, 20C, and 20D show the effect that the order in which the different patient's clinical information are applied has on the diagnostic accuracy of the algorithms disclosed herein for diagnosing amyloid-β accumulation based on miR-485-3p expression in human clinical swab samples from patients over 60 years old. FIG. 20A provides a comparison of the average AUC (area under the curve) values generated for the following algorithms constructed using various combinations of different diagnostic parameters: (i) miR-485-3p expression, age, gender, and education year (“pre-DX”); (ii) mir-485-3p expression, age, gender, education year, APOE genotype, and MMSE score (“pro-DX1”); and (iii) miR-485-3p expression, age, gender, education year, APOE genotype, MMSE score, and CDR score (“pro-DX2”). “AUC” refers to the area under curve resulting from a comparison of the specificity and sensitivity values. AUC was measured by ROC analysis. FIGS. 20B, 20C, and 20D provide the accuracy data for the different algorithms shown in FIG. 20A, i.e., pre-DX, pro-DX1, and pro-DX2, respectively. In each of FIGS. 20B, 20C, and 20D, the y-axis provides the different types of clinical information that were combined with the miR-485-3p expression. The arrow represents the most accurate combination. The information provided within the boxed region represents the specific order in which the different clinical information of the most accurate combination (i.e., represented by the red arrow) was applied to the algorithms. The y-axis in FIGS. 20B, 20C, and 20D provides the type of clinical information applied to the algorithm along with the quantitative value of miR-485-3p expression. The applied clinical information was divided into underbars.

FIGS. 21A, 21B, and 21C show the effect that the order in which the different patient's clinical information are applied has on the diagnostic accuracy of the algorithms disclosed herein for diagnosing amyloid-β accumulation based on miR-485-3p expression in human clinical swab samples from patients less than 61 years old. FIG. 21A provides the accuracy data determined using the pre-DX algorithm. FIG. 21B provides the accuracy data determined using the pro-DX1 algorithm. FIG. 21C provides the accuracy data determined using the pro-DX2 algorithm. In each of the figures, the y-axis provides the different types of clinical information that were combined with the miR-485-3p expression. The arrow represents the most accurate combination. The information provided within the boxed region represents the specific order in which the different clinical information of the most accurate combination (i.e., represented by the red arrow) was applied to the algorithms.

FIGS. 22A and 22B provide comparison of AUC and accuracy values, respectively, for the following algorithms: (i) pre-DX, (ii) pro-DX1, and (iii) pro-DX2. FIG. 22C provides a comparison of how the different clinical information affects the accuracy of the algorithms. The effect on accuracy is shown as the accuracy correction rate, which compares the accuracy of the algorithms with and without the specific clinical information indicated along the x-axis. The results shown in FIGS. 22A, 22B, and 22C are based on samples from patients who are 61 years old and older. FIGS. 22D and 22E provide the same results shown in FIGS. 22A and 22B, respectively, except the results are based on samples from all patients (i.e., not limited to certain age group). FIG. 22F provide the same results shown in FIG. 22C except the results are based on samples from all patients (i.e., not limited to certain age group).

FIGS. 23A and 23B show the sensitivity, specificity, and AUC values in patient's oral swab samples after K-fold cross validation. The values were determined using one of the following algorithms: (i) pre-DX (left graph), (ii) pro-DX1 (middle graph), and (iii) pro-DX2 (right graph). The results provides in FIGS. 23A and 23B are from two independent experiments.

FIGS. 24A, 24B, 24C, and 24D show the diagnostic score (bar graph to the left) for clinical swab samples from patients at least 61 years in age. The score was generated using regression modeling based on a combination of the following diagnostic parameters: (i) miR-485-3p expression and age (FIG. 24A); (ii) miR-485-3p expression, age, gender, and education year (referred to herein as “pre-DX”) (FIG. 24B); (iii) mir-485-3p expression, age, gender, education year, APOE genotype, and MMSE score (referred to herein as “pro-DX1”) (FIG. 24C); and (iv) miR-485-3p expression, age, gender, education year, APOE genotype, MMSE score, and CDR score (referred to herein as “pro-DX2”) (FIG. 24D). Each of the patient samples were categorized based on amyloid-β accumulation: (i) without amyloid-β accumulation (left bar, i.e., amyloid PET negative) (n=19); or (ii) with amyloid-β accumulation (right bar, i.e., amyloid PET positive) (n=20). To quantify the miR-485-3p expression, the real-time PCR was repeated 4.3 times on average. The horizontal gray box crossing the box plot represents the gray zone. The gray zone was the section that adds and subtracts the half of the standard deviation of the score for each sample to the first cutoff value. In each of FIGS. 24A, 24B, 24C, and 24D, the ROC graph to the right shows the specificity (y-axis) and sensitivity (x-axis) of using the different combinations of diagnostic parameters described above. Receiver Operating Characteristic (ROC) analysis was used to measure the following values: (i) area under the curve (AUC), (ii) sensitivity, (iii) specificity, and (iv) accuracy. The statistical values of the ROC analysis were generated by excluding the gray zone results. Drop out ratio was the ratio of the results not included in the gray zone among the total results. The circular point identified by the arrow represents the specificity and sensitivity at which the accuracy was the highest among the results excluding the gray zone.

FIGS. 25A, 25B, 25C, and 25D are the same results shown in FIGS. 24A, 24B, 24C, and 24D, respectively, except that the results are based on samples from all patients (i.e., not restricted to any specific age group.)

FIGS. 26A, 26B, and 26C show the ability of combining miR-485 expression from human clinical swab samples with various clinical information to diagnose patients with the following cognitive impairments: (i) normal cognitive (“NC”); (ii) mild cognitive impairment (“MCI”), and (iii) Alzheimer's disease (“AD”). FIG. 26A provides a comparison of the diagnostic scores generated using regression modeling based on a combination of the following parameters: (i) miR-485-3p expression and age (first bar graph from the left; “quantity”); (ii) miR-485-3p expression, age, gender, and education year (second bar graph from the left; “pre-DX”); (iii) mir-485-3p expression, age, gender, education year, APOE genotype, and MMSE score (third bar graph from the left; “pro-DX1”); and (iv) miR-485-3p expression, age, gender, education year, APOE genotype, MMSE score, and CDR score (fourth bar graph from the left; “pro-DX2”). In each of the “NC” and “MCI” groups shown, the box to the left represents samples from patients without amyloid-β accumulation (i.e., amyloid PET negative), and the box to the right represents samples from patients with amyloid-β accumulation (i.e., amyloid PET positive). In the “AD” groups, all patients were positive for amyloid-β accumulation (i.e., amyloid PET positive). The Q values provided refer to the minus log 10 of the p-value as measured using student's t-test. The horizontal gray box crossing the box plot represents the gray zone. The gray zone was the section that adds and subtracts the half of the standard deviation of the score for each sample to the first cutoff value (see method). Each circle represents an individual sample. FIG. 26B provides a comparison of the following values for prediction of amyloid-β accumulation based on cognitive impairment diagnosis (i.e., diagnosed as either having normal impairment (“NC”) or mild cognitive impairment (“MCI”): (i) accuracy, (ii) AUC, (iii) sensitivity, and (iv) specificity. FIG. 26C provides the specificity (y-axis) and sensitivity (x-axis) values generated based on the combination of diagnostic parameters described in FIG. 26A, i.e., (i) “quantity” (first column); (ii) “pre-DX” (second column); (iii) “pro-DX1” (third column); and (iv) “pro-DX2” (fourth column). The top row shows the results for clinical swab samples from patients diagnosed as having normal impairment (“NC”), and the bottom row shows the results for clinical swab samples from patients diagnosed as having mild cognitive impairment (“MCI”). The shaded area in each of the graphs shown in FIG. 26C represents the AUC as measured by ROC analysis. The circular point identified by the arrow represents the specificity and sensitivity at which the accuracy was the highest among the results excluding the gray zone.

### DETAILED DESCRIPTION OF THE DISCLOSURE

The present disclosure is generally directed to identifying a subject (e.g., human subject) afflicted with a cognitive disorder, comprising measuring the subject's miR-485-3p level (e.g., in a biological sample, e.g., extracellular vesicles, derived from the subject). In some aspects, the methods disclosed herein further comprises administering a miR-485-3p inhibiter therapy to a subject identified as being afflicted with a cognitive disorder. The miR-485 inhibitor comprises a nucleotide sequence encoding a nucleotide molecule that comprises at least one miR-485 binding site, wherein the nucleotide molecule does not encode a protein. In some aspects, the miRNA binding site or sites can bind to endogenous miR-485, which inhibits and/or reduces the expression level and/or activity of miR-485-3p in the subject.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV” includes a derivative of a known AAV. In some aspects, an “AAV” includes a modified or an artificial AAV.

The terms “administration,” “administering,” and grammatical variants thereof refer to introducing a composition, such as a miRNA inhibitor of the present disclosure, into a subject via a pharmaceutically acceptable route. The introduction of a composition, such as a micelle comprising a miRNA inhibitor of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “afflicted with” can be used interchangeably with the term “suffering from” and refers to the state of having a cognitive disorder disclosed herein. In some aspects, a subject afflicted with a cognitive disorder exhibits one or more symptoms associated with the cognitive disorder (e.g., loss of memory for Alzheimer's disease patients). However, as will be apparent to those skilled in the art, a subject does not need to exhibit one or more symptoms to be afflicted with a disease or disorder disclosed herein (e.g., can have a genetic predisposition to the disease or disorder).

As used herein, the term “associated with” refers to a close relationship between two or more entities or properties. For instance, when used to describe a disease or condition that can be diagnosed with the present disclosure (e.g., disease or condition associated with an abnormal level of a miRNA, e.g., miR-485-3p), the term “associated with” refers to an increased likelihood that a subject suffers from (i.e., afflicted with) the disease or condition when the subject exhibits an abnormal miRNA (e.g., miR-485-3p) expression level. In some aspects, the abnormal expression causes the disease or condition. In some aspects, the abnormal expression does not necessarily cause but is correlated with the disease or condition. Non-limiting examples of suitable methods that can be used to determine whether a subject exhibits an abnormal expression of a protein and/or gene associated with a disease or condition are provided elsewhere in the present disclosure.

As used herein, the term “abnormal level” refers to a level (expression and/or activity) that differs (e.g., increased) from a reference subject who does not suffer from a disease or condition described herein (e.g., cognitive disorder). In some aspects, an abnormal level (e.g., of miR-485-3p) refers to a level that is increased by at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 2-fold, at least about 3-fold, at least about 4-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, at least about 750-fold, or at least about 1,000-fold or more compared to the corresponding level in a reference subject (e.g., subject who does not suffer from a disease or condition described herein).

As used herein, the term “cognitive disorder” refers to any disorder that affects mental processes, including, but not limited to, impairments in memory, learning, awareness, attention, communication, motor coordination, and/or intellectual capacity. In some aspects, the cognitive disorder is Alzheimer's disease (AD) and/or Mild Cognitive Impairment (MCI). In some aspects, a “cognitive disorder” refers to AD, MCI, amnesia, corticobasal syndrome, dementia, lewy body dementia, frontotemporal dementia, primary progressive aphasia, progressive nonfluent aphasia, progressive supranuclear palsy, pseudosenility, semantic dementia, severe cognitive impairment, subcortical dementia, vascular dementia, amyotrophic lateral sclerosis (ALS), and/or logopenic progressive aphasia. In some aspects, the cognitive disorder is associated with amyloid-β accumulation.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence can apply to the entire length of a polynucleotide or polypeptide or can apply to a portion, region or feature thereof.

The term “derived from,” as used herein, refers to a component that is isolated from or made using a specified molecule or organism, or information (e.g., amino acid or nucleic acid sequence) from the specified molecule or organism. For example, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. In some aspects, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′->3′),” is complementary to the nucleobase sequence “A-C-T (3′->5′).” Complementarity can be “partial,” in which less than all of the nucleobases of a given nucleobase sequence are matched to the other nucleobase sequence according to base pairing rules. For example, in some aspects, complementarity between a given nucleobase sequence and the other nucleobase sequence can be about 70%, about 75%, about 80%, about 85%, about 90%, or about 95%. Accordingly, in certain aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a target nucleic acid sequence (e.g., miR-485 nucleic acid sequence). Or, there can be “complete” or “perfect” (100%) complementarity between a given nucleobase sequence and the other nucleobase sequence to continue the example. In some aspects, the degree of complementarity between nucleobase sequences has significant effects on the efficiency and strength of hybridization between the sequences.

As used herein, the term “diagnosing” (and derivatives thereof) refers to methods that can be used to determine or predict whether a subject is afflicted with, suffering from, or at a risk (e.g., genetically predisposed) for a given disease or condition, thereby identifying a subject who is suitable for a treatment. In some aspects, the treatment can be therapeutic (e.g., administered to a subject exhibiting one or more symptoms associated with the disease or disorder). In some aspects, the treatment can be prophylactic (e.g., administered to an at-risk subject to prevent and/or reduce the onset of the disease or disorder). As described herein, a skilled artisan can make a diagnosis on the basis of one or more diagnostic marker (e.g., miR-485-3p), where the presence, absence, amount, or change in the amount of the diagnostic marker is indicative of the presence, severity, or absence of the condition. In some aspects, an increase in miR-485-3p expression (e.g., in a biological sample from the subject) is indicative of a cognitive disorder (e.g., Alzheimer's disease). The term “diagnosis” does not refer to the ability to determine the presence or absence of a particular disease or disorder with 100% accuracy, or even that a given course or outcome is more likely to occur than not. Instead, the skilled artisan will understand that the term “diagnosis” refers to an increased probability that a certain disease or disorder is present in the subject. In some aspects, the term “diagnosis” includes one or more diagnostic methods of identifying a subject who has a cognitive disorder (e.g., those described herein).

The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription.

The terms “excipient” and “carrier” are used interchangeably and refer to an inert substance added to a pharmaceutical composition to further facilitate administration of a compound, e.g., a miRNA inhibitor of the present disclosure.

The term “expression,” as used herein, refers to a process by which a polynucleotide produces a gene product, e.g., RNA or a polypeptide. It includes without limitation transcription of the polynucleotide into micro RNA binding site, small hairpin RNA (shRNA), small interfering RNA (siRNA), or any other RNA product. It includes, without limitation, transcription of the polynucleotide into messenger RNA (mRNA), and the translation of mRNA into a polypeptide. Expression produces a “gene product.” As used herein, a gene product can be, e.g., a nucleic acid, such as an RNA produced by transcription of a gene. As used herein, a gene product can be either a nucleic acid, RNA or miRNA produced by the transcription of a gene, or a polypeptide which is translated from a transcript. Gene products described herein further include nucleic acids with post transcriptional modifications, e.g., polyadenylation or splicing, or polypeptides with post translational modifications, e.g., phosphorylation, methylation, glycosylation, the addition of lipids, association with other protein subunits, or proteolytic cleavage. As used herein, the term “expression” can be used interchangeable with the term “level.” For instance, in some aspects, the term “miR-485-3p expression” can be synonymous with the term “miR-485-3p level.”

As used herein, the term “extracellular vesicle” (EV) refers to a cell-derived vesicle comprising a membrane that encloses an internal space. Extracellular vesicles comprise all membrane-bound vesicles (e.g., exosomes, nanovesicles, microvesicles) that have a smaller diameter than the cell from which they are derived. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are derived from oral epithelial cells (e.g., from human clinical swab samples). In some aspects, the extracellular vesicles are derived from serum/plasma (e.g., from human plasma samples). In some aspects, an extracellular vesicle comprises an exosome, microvesicle, nanovesicle, or combinations thereof. In certain aspects, an extracellular vesicle is an exosome.

As used herein, the term “exosome” refers to a cell-derived vesicle having a diameter of between about 20 nm to about 300 nm. Exosomes include specific surface markers not present in other vesicles, including surface markers such as tetraspanins, e.g. CD9, CD37, CD44, CD53, CD63, CD81, CD82 and CD151; targeting or adhesion markers such as integrins, ICAM-1, EpCAM and CD31; membrane fusion markers such as annexins, TSG101, ALIX; and other exosome transmembrane proteins such as Rab5b, HLA-G, HSP70, LAMP2 (lysosome-associated membrane protein) and LIMP (lysosomal integral membrane protein).

As used herein, the term “microvesicle” (MV) refers to a type of EV (i.e., cell-derived vesicle) with a diameter larger than exosomes. In some aspects, microvesicles comprise a diameter of between about 10 nm to about 5,000 nm (e.g., between about 50 nm and 1500 nm, between about 75 nm and 1500 nm, between about 75 nm and 1250 nm, between about 50 nm and 1250 nm, between about 30 nm and 1000 nm, between about 50 nm and 1000 nm, between about 100 nm and 1000 nm, between about 50 nm and 750 nm, etc.).

As used herein, the term “nanovesicle” refers to a cell-derived vesicle having a diameter of between about 20 nm to about 250 nm (e.g., between about 30 nm to about 150 nm).

As used herein, the term “human oral-derived cell free exosome” (HOCFE) refer to nanosized bodies (e.g., exosomes) isolated from human oral bio-fluid. Exemplary methods of isolating HOCFE from human swab samples are provided elsewhere in the present disclosure.

As used herein, the term “homology” refers to the overall relatedness between polymeric molecules, e.g. between nucleic acid molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the monomers in the molecule are identical (exactly the same monomer) or are similar (conservative substitutions). The term “homologous” necessarily refers to a comparison between at least two sequences (e.g., polynucleotide sequences).

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “identity” refers to the overall monomer conservation between polymeric molecules, e.g., between polynucleotide molecules. The term “identical” without any additional qualifiers, e.g., polynucleotide A is identical to polynucleotide B, implies the polynucleotide sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions, or bases in the case of polynucleotides, are then compared.

When a position in the first sequence is occupied by the same amino acid or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs that can be used to align different sequences (e.g., polynucleotide sequences) are available from various sources. One suitable program to determine percent sequence identity is bl2seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at worldwideweb.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure, e.g., a miRNA inhibitor of the present disclosure, that has undergone one or more processes of purification. In some aspects, isolating or purifying as used herein is the process of removing, partially removing (e.g., a fraction) of a composition of the present disclosure, e.g., a miRNA inhibitor of the present disclosure from a sample containing contaminants.

In some aspects, an isolated composition has no detectable undesired activity or, alternatively, the level or amount of the undesired activity is at or below an acceptable level or amount. In other aspects, an isolated composition has an amount and/or concentration of desired composition of the present disclosure, at or above an acceptable amount and/or concentration and/or activity. In other aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.99990%, or greater than 99.9999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

A “miRNA inhibitor,” as used herein, refers to a compound that can decrease, alter, and/or modulate miRNA expression, function, and/or activity. The miRNA inhibitor can be a polynucleotide sequence that is at least partially complementary to the target miRNA nucleic acid sequence, such that the miRNA inhibitor hybridizes to the target miRNA sequence. For instance, in some aspects, a miR-485-3p inhibitor comprises a nucleotide sequence encoding a nucleotide molecule that is at least partially complementary to the target miR-485-3p nucleic acid sequence, such that the miR-485-3p inhibitor hybridizes to the miR-485-3p sequence. In some aspects, the hybridization of the miR-485-3p inhibitor to the miR-485-3p sequence decreases, alters, and/or modulates the expression, function, and/or activity of miR-485-3p.

The terms “miRNA,” “miR,” and “microRNA” are used interchangeably and refer to a microRNA molecule found in eukaryotes that is involved in RNA-based gene regulation. The term will be used to refer to the single-stranded RNA molecule processed from a precursor. In some aspects, the term “antisense oligomers” can also be used to describe the microRNA molecules of the present disclosure. Names of miRNAs and their sequences related to the present disclosure are provided herein. MicroRNAs recognize and bind to target mRNAs through imperfect base pairing leading to destabilization or translational inhibition of the target mRNA and thereby downregulate target gene expression. Conversely, targeting miRNAs via molecules comprising a miRNA binding site (generally a molecule comprising a sequence complementary to the seed region of the miRNA) can reduce or inhibit the miRNA-induced translational inhibition leading to an upregulation of the target gene.

The terms “mismatch” or “mismatches” refer to one or more nucleobases (whether contiguous or separate) in an oligomer nucleobase sequence (e.g., miR-485-3p inhibitor) that are not matched to a target nucleic acid sequence (e.g., miR-485-3p) according to base pairing rules. While perfect complementarity is often desired, in some aspects, one or more (e.g., 6, 5, 4, 3, 2, or 1 mismatches) can occur with respect to the target nucleic acid sequence. Variations at any location within the oligomer are included. In certain aspects, antisense oligomers of the disclosure (e.g., miR-485-3p inhibitor) include variations in nucleobase sequence near the termini, variations in the interior, and if present are typically within about 6, 5, 4, 3, 2, or 1 subunits of the 5′ and/or 3′ terminus. In some aspects, one, two, or three nucleobases can be removed and still provide on-target binding.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances, a modulator can increase and/or decrease a certain concentration, level, activity or function relative to a control, or relative to the average level of activity that would generally be expected or relative to a control level of activity. In some aspects, a miRNA-485-3p inhibitor disclosed herein can modulate (e.g., decrease, alter, or abolish) miR-485-3p expression, function, and/or activity.

The term “naïve,” as used herein to describe cycle threshold (Ct) refers to raw Ct values (i.e., as measured directly from the PCR assay and without any further calculation).

“Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

The terms “pharmaceutically acceptable carrier,” “pharmaceutically acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., a miRNA inhibitor of the present disclosure, mixed or intermingled with, or suspended in one or more other chemical components, such as pharmaceutically acceptable carriers and excipients. One purpose of a pharmaceutical composition is to facilitate administration of preparations comprising a miRNA inhibitor of the present disclosure to a subject.

The term “polynucleotide,” as used herein, refers to polymers of nucleotides of any length, including ribonucleotides, deoxyribonucleotides, analogs thereof, or mixtures thereof.

In some aspects, the term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide.

In some aspects, the term “polynucleotide” includes polydeoxyribonucleotides (containing 2-deoxy-D-ribose), polyribonucleotides (containing D-ribose), including tRNA, rRNA, shRNA, siRNA, miRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some aspects of the present disclosure, a polynucleotide can be, e.g., an oligonucleotide, such as an antisense oligonucleotide. In some aspects, the oligonucleotide is an RNA. In some aspects, the RNA is a synthetic RNA. In some aspects, the synthetic RNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine).

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function.

Polypeptides include gene products, naturally occurring polypeptides, synthetic polypeptides, homologs, orthologs, paralogs, fragments and other equivalents, variants, and analogs of the foregoing.

A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to about 50 amino acids long, e.g., about 5, about 10, about 15, about 20, about 25, about 30, about 35, about 40, about 45, or about 50 amino acids long.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, the terms “promoter” and “promoter sequence” are interchangeable and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. In some aspects, a promoter that can be used with the present disclosure includes a tissue specific promoter.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent the onset of a disease or condition, or to prevent or delay a symptom associated with a disease or condition.

As used herein, the term “gene regulatory region” or “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, or stem-loop structures. If a coding region is intended for expression in a eukaryotic cell, a polyadenylation signal and transcription termination sequence will usually be located 3′ to the coding sequence.

In some aspects, a miR-485-3p inhibitor disclosed herein (e.g., a polynucleotide encoding a RNA comprising one or more miR-485-3p binding site) can include a promoter and/or other expression (e.g., transcription) control elements operably associated with one or more coding regions. In an operable association a coding region for a gene product is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control elements, besides a promoter, for example enhancers, operators, repressors, and transcription termination signals, can also be operably associated with a coding region to direct gene product expression.

As used herein, the term “similarity” refers to the overall relatedness between polymeric molecules, e.g. between polynucleotide molecules (e.g. miRNA molecules). Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the nucleic acids are compared, e.g., according to their evolutionary proximity, charge, volume, flexibility, polarity, hydrophobicity, aromaticity, isoelectric point, antigenicity, or combinations thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the term “therapeutically effective amount” is the amount of reagent or pharmaceutical compound comprising a miRNA inhibitor of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refers to, e.g., the reduction in severity of a disease or condition; the reduction in the duration of a disease course; the amelioration or elimination of one or more symptoms associated with a disease or condition (e.g., diabetes); the provision of beneficial effects to a subject with a disease or condition, without necessarily curing the disease or condition. The term also includes prophylaxis or prevention of a disease or condition or its symptoms thereof.

The term “upstream” refers to a nucleotide sequence that is located 5′ to a reference nucleotide sequence.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

### II. Diagnostic Methods

Disclosed herein are methods of diagnosing a cognitive disorder in a subject in need thereof. In some aspects, such methods comprise identifying a subject afflicted with a cognitive disorder. Not to be bound by any one theory, Applicant has identified that subjects with certain cognitive disorders have higher level of miR-485-3p compared to those subjects not suffering from a cognitive disorder. Accordingly, in some aspects, the present disclosure provides a method of identifying a subject (e.g., human subject) afflicted with a cognitive disorder, wherein the method comprises measuring a subject's miR-485-3p level, wherein an increase in the subject's miR-485-3p level compared to a reference (e.g., corresponding value in a subject not suffering from a cognitive disorder or corresponding value in the subject prior to the onset of the cognitive disorder) suggests that the subject is afflicted with the cognitive disorder.

In some aspects, the level of miR-485-3p in the subject is increased by at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, at least about 300%, at least about 400%, at least about 500%, at least about 1,000% or more, compared to the reference (e.g., corresponding value in a subject not suffering from a cognitive disorder or corresponding value in the subject prior to the onset of the cognitive disorder).

As described herein, in some aspects, the miR-485-3p level is measured in a biological sample of the subject. Accordingly, in some aspects, a method of identifying a subject afflicted with a cognitive disorder comprises obtaining a biological sample from the subject prior to measuring the miR-485-3p expression. In some aspects, a biological sample comprises any cell, tissue, and/or fluid of a subject that can be used to measure the expression level of a molecule of interest (e.g., miR-485-3p). In some aspects, a biological sample comprises a tissue, cell, blood, serum, plasma, saliva, cerebrospinal fluid, intravitreal fluid, urine, or combinations thereof. In some aspects, the biological sample is derived from an epithelial cell of the subject. In certain aspects, the epithelial cell comprises oral epithelial cells, e.g., such as those that can be obtained through a swab sample. In some aspects, the biological sample is derived from a subject's serum and/or plasma.

MicroRNAs (miRNAs) are present not only in many mammalian cell types (e.g., oral epithelial cells) but can also be transported through bodily fluids within extracellular vesicles (e.g., exosomes). Once released into the extracellular fluid, exosomes fuse with other cells and can transfer their cargo to the recipient cells. Accordingly, in some aspects, the biological sample in which the miR-485-3p expression can be measured comprises an extracellular vesicle. In certain aspects, the extracellular vesicle comprises a microvesicle. In certain aspects, the extracellular vesicle comprises an exosome. In some aspects, the extracellular vesicle comprises a nanovesicle.

As described herein, Applicant has shown a positive correlation between miR-485-3p expression level and one or more characteristics of a cognitive disorder. For instance, in some aspects, an increase in miR-485-3p expression is associated with an increase in amyloid-β accumulation, which can lead to the formation of amyloid-β plaques in the subject. In some aspects, the greater the miR-485-3p expression level, the greater the amyloid-β accumulation in the subject.

In some aspects, a method of diagnosing a cognitive disorder can comprise assessing the presence of (e.g., measuring) one or more characteristics of a cognitive disorder in a subject. Accordingly, in some aspects, the present disclosure provides a method of measuring one or more characteristics of a cognitive disorder in a subject in need thereof, comprising measuring the subject's miR-485-3p level, wherein the subject's miR-485-3p level is positively correlated with the one or more characteristics of the cognitive disorder. In certain aspects, the presence of one or more characteristics of a cognitive disorder indicates that the subject is afflicted with the cognitive disorder. In some aspects, the one or more characteristics of a cognitive disorder comprises an accumulation of amyloid-β. Accordingly, in some aspects, the diagnostic methods disclosed herein can be useful in identifying subjects afflicted with a cognitive disorder that is associated with amyloid-β accumulation. Non-limiting examples of such cognitive disorders include Alzheimer's Disease (AD), frontotemporal dementia (FTD), cerebrovascular dementia (CVD), mild cognitive impairment (MCI), dementia with Lewy Bodies (DLB), and combinations thereof.

In some aspects, the methods disclosed herein can be used to identify a subject afflicted with an Alzheimer's disease. In certain aspects, Alzheimer's disease comprises pre-dementia Alzheimer's disease, early Alzheimer's disease, moderate Alzheimer's disease, advanced Alzheimer's disease, early onset familial Alzheimer's disease, inflammatory Alzheimer's disease, non-inflammatory Alzheimer's disease, cortical Alzheimer's disease, early-onset Alzheimer's disease, late-onset Alzheimer's disease, or any combination thereof.

As will be apparent to those skilled in the art, a subject's miR-485-3p expression can be measured by various means known in the art. Non-limiting examples of assays that can be used to measure miR-485-3p expression include PCR (e.g., real-time PCR), Northern blot, liquid chromatography-mass spectrometry (LC-MS), mass spectrometry (MS), next-generation sequencing (NGS) (e.g., Ion Torrent), nanostring, microarray, ELISA (aptamer), RNA immunoprecipitation (RIP), RNA in situ hybridization, RNA fluorescence in situ hybridization (FISH), and combinations thereof. In some aspects, miR-485-3p expression can be measured using a polymerase chain reaction (PCR) assay. When using a PCR assay, in some aspects, any of the primers provided in Table 1 (below) can be used to measure the miR-485-3p expression. In some aspects, the miR-485-3p primer comprises miR-485-3p_FW7. In some aspects, the miR-485-3p primer comprises miR-485-3p_FW2. In some aspects, the miR-485-3p primer comprises miR-485-3p_FW1. In some aspects, the miR-485-3p primer comprises miR-485-3p_FW9.

TABLE 1
miR-485-3p primer sequences

| Oligonucleotide Name | Sequence (5′ to 3′) |
|---|---|
| miR-485-3p_FW1 | GTCATACACGGCTCTCCTCTCT |
| | (SEQ ID NO: 94) |
| miR-485-3p_FW2 | TCATACACGGCTCTCCTCTC |
| | (SEQ ID NO: 95) |
| miR-485-3p_FW3 | CATACACGGCTCTCCTCTC |
| | (SEQ ID NO: 96) |
| miR-485-3p_FW4 | CATACACGGCTCTCCTCTCTA |
| | (SEQ ID NO: 97) |
| miR-485-3p_FW5 | CATACACGGCTCTCGTCTC |
| | (SEQ ID NO: 98) |
| miR-485-3p_FW6 | CATACACGGCTCTCGTCTCTAA |
| | (SEQ ID NO: 99) |
| miR-485-3p_FW7 | GTCATACACGGCTCTCCTCTCTAA |
| | (SEQ ID NO: 100) |
| miR-485-3p_FW8 | GTCATACACGGCTCTCCTC |
| | (SEQ ID NO: 101) |
| miR-485-3p_FW9 | CATACACGGCTCTCCTCTCTAAA |
| | (SEQ ID NO: 52) |
| miR-485-3p_FW10 | GTCATACACGGCTCTCCTCTG |
| | (SEQ ID NO: 102) |
| miR-485-3p_FW11 | TCATACACGGCTCTCCTCTCT |
| | (SEQ ID NO: 103) |
| miR-485-3p_FW12 | TCATACACGGCTCTCCTC |
| | (SEQ ID NO: 104) |
| miR-485-3p_FW13 | TCATACACGGCTCTCCTCTCTAA |
| | (SEQ ID NO: 105) |
| miR-485-3p_FW14 | CATACACGGCTCTCCTCTCTAA |
| | (SEQ ID NO: 106) |
| miR-485-3p_FW15 | ATACACGGCTCTCCTCTCTAA |
| | (SEQ ID NO: 107) |

In some aspects, the expression of miR-485-3p (e.g., in a biological sample of a subject) is measured using a real-time PCR assay. In such aspects, miR-485-3p expression can be assessed by determining the cycle threshold (Ct) number for miR-485-3p. As used herein, the term “cycle threshold” (Ct) refers to the cycle number (i.e., number of amplifications) during thermal cycling of the real-time PCR assay at which the amount of fluorescence due to product formation reaches a fixed threshold value above a baseline value (i.e., exceeds background level). Ct levels are inversely proportional to the level of miR-485-3p present in the sample (i.e., the lower the Ct level, the greater the level of miR-485-3p present in the sample).

In some aspects, the Ct number in a subject afflicted with a cognitive disorder (e.g., those described herein) is decreased by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% or more, compared to a reference (e.g., corresponding value in a subject not suffering from a cognitive disorder or corresponding value in the subject prior to the onset of the cognitive disorder).

Not to be bound by any one theory, in some aspects, the diagnostic methods disclosed herein can identify a subject afflicted with a cognitive disorder based on an association between miR-485-3p expression and the presence of one or more characteristics of a cognitive disorder (e.g., amyloid-β accumulation) in the subject. In some aspects, the level of miR-485-3p and the presence of one or more characteristics of a cognitive disorder is positively correlated. In certain aspects, the higher presence of the one or more characteristics is indicative of the severity of the cognitive disorder. Accordingly, in some aspects, the present disclosure relates to a method of determining the severity of a cognitive disorder in a subject in need thereof, comprising measuring the subject's miR-485-3p level, wherein the miR-485-3p level is positively correlated with severity.

As described herein, in some aspects, combining a subject's miR-485-3p level with one or more additional clinical information about the subject can improve the diagnostic accuracy of using miR-485-3p expression to identify a subject afflicted with a cognitive disorder. Non-limiting examples of additional clinical information that can be used include age, gender, education year (or level), apoliprotein E (APOE) genotype, mini mental state examination (MMSE) score, cognitive impairment, clinical dementia rating (CDR) score, and combinations thereof.

To combine one or more of the additional clinical information with a subject's miR-485-3p level, a numerical value is established for the different clinical information (see, e.g., Example 3). In some aspects, the additional clinical information is age, and the value associated with age is the age of the subject at the time of measuring the miR-485-3p expression. In some aspects, the additional clinical information is the patient's gender, wherein male is associated with a value of “1” and female is associated with a value of “2.” In some aspects, the additional clinical information is the patient's total educational year, which is associated with a value ranging from 0-16. For each year of school (i.e., elementary/primary school, middle school, high school, and college) completed, the patient receives a value of “1” for educational year. For elementary/primary school, a patient can receive a value from 1 to 6 (i.e., 1^{st} to 6^{th} grade). For middle school, a patient can receive an additional value from 1 to 3 (i.e., 7^{th} to 9^{th} grade). For high school, a patient can receive an additional value from 1 to 3 (i.e., 10^{th} to 11^{th} grade). For college, a patient can receive an additional value from 1 to 4. For instance, a patient who graduated from a 4-year college would receive the maximum value of 16 for educational year. A patient who graduated from high school but did not attend college would receive a value of 12 for education year. A patient who did not attend elementary/primary school and beyond would receive a value of 0 for educational year.

In some aspects, the additional clinical information is the patient's APOE genotype, wherein (i) E2/E3=1; (ii) E3/E3=1; (iii) E2/E4=2; (iv) E3/E4=2; and (iv) E4/E4=4. As used herein, the term “apoliprotein E” (APOE) refers to one of the five main types of blood lipoproteins (A-E). The APOE gene exists in three different forms (alleles)—E2, E3, and E4. All human subjects inherit a pair of APOE genes that is some combination of these three. APOE e4 has been described as being associated with an increased risk of late onset Alzheimer's disease (i.e., that develop after the age of 65). Liu et al., Nat Rev Neurol 9(2): 106-118 (February 2013).

In some aspects, the additional clinical information is the subject's Mini-Mental State Examination (MMSE) (also known as the Folstein test) score. The term “Mini-Mental State Examination” (MMSE) refers to a 30-point questionnaire that is used extensively in clinical and research settings to measure cognitive impairment. Arevalo-Rodriguez et al., Cochrane Database Syst Rev (3): CD010783 (March 2015). In some aspects, the MMSE score is based on the categories shown in Table 2 (below). In certain aspects, MMSE score of 24 points or higher indicates normal cognition. In some aspects, MMSE score of <9 points indicates severe cognitive impairment. In some aspects, MMSE score of 10-18 points indicates moderate cognitive impairment. In some aspects, MMSE score of 19-23 points indicates mild cognitive impairment.

TABLE 2
Mini-Mental State Examination (MMSE) Categories

| | Possible | |
|---|---|---|
| Category | Points | Description |
| Orientation to | 5 | From broadest to most narrow. Orientation to time has |
| time | | been correlated with future decline. |
| Orientation to | 5 | From broadest to most narrow. This is sometimes |
| place | | narrowed down to streets, and sometimes to floor. |
| Registration | 3 | Repeating named prompts |
| Attention and | 5 | Serial sevens, or spelling “world” backwards. It has |
| calculation | | been suggested that serial sevens can be more |
| | | appropriate in a population where English is |
| | | not the first language |
| Recall | 3 | Registration recall |
| Language | 2 | Naming a pencil and a watch |
| Repetition | 1 | Speaking back a phrase |
| Complex | 6 | Varies. Can involve drawing figure shown |
| commands | | |

In some aspects, to identify a subject afflicted with a cognitive disorder, a subject's miR-485-3p expression level is used in combination with one, two, three, four, or all five of the additional clinical information described above (i.e., age, gender, education year, APOE genotype, and MMSE score).

In some aspects, a subject's miR-485-3p expression is used in combination with all five of the additional clinical information described above. In such aspects, the diagnostic accuracy of the combination can be assessed by calculating a score for a given biological sample using the following formula: (Naïve CT×(Age×V1_{Age}+V2_{Age}))×(Gender×V1_{Gender}+V2_{Gender})×(APOE×V1_{APOE}+V2_{APOE})×(MMSE×V1_{MMSE}+V2_{MMSE})×(Education year×V1_{EDU}+V2_{EDU}), wherein V1 and V2 are regression coefficient values (i.e., slope and intercept of the regression curve, respectively) associated with the specific additional clinical information.

In some aspects, a subject's miR-485-3p expression is used in combination with two of the additional clinical information described above. In certain aspects, the additional clinical information include gender and education year. In such aspects, the diagnostic accuracy of the combination can be assessed by calculating a score for a given biological sample using the following formula: (Naïve Ct×(Gender×V1_{Gender}+V2_{Gender}))×(Education year×V1_{EDU}+V2_{EDU}), wherein V1 and V2 are regression coefficient values associated with the specific additional clinical information.

In some aspects, a subject's miR-485-3p expression is used in combination with one additional clinical information described above. In certain aspects, the additional clinical information is gender. In such aspects, the diagnostic accuracy of the combination can be assessed by calculating a score for a given biological sample using the following formula: (Naïve CT×(Gender×V1_{Gender}+V2_{Gender})), wherein V1 and V2 are regression coefficient values associated with the specific additional clinical information.

In some aspects, the diagnostic accuracy of the combination of miR-485-3p expression and one or more of the clinical information described herein can be assessed using any of the equations (also referred to herein as a formula or an algorithm) provided in Table 9.

In some aspects, the score for a biological sample derived from a subject afflicted with a cognitive disorder in any one of the combinations described above is less than the corresponding score of a reference sample (e.g., from a subject not suffering from a cognitive disorder or from the subject prior to the onset of the cognitive disorder). In certain aspects, the score for a biological sample from a subject afflicted with a cognitive disorder is less than at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% or more, compared to the corresponding score of the reference sample.

In some aspects, the diagnostic methods disclosed herein can be used in combination with other methods for diagnosing a cognitive disorder. Non-limiting examples of such additional methods include brain scans, such as computed tomography (CT), magnetic resonance imaging (MRI), or positron emission tomography (PET). In some aspects, the diagnostic methods disclosed herein can be used to rule out other medical conditions that can cause similar symptoms as the cognitive disorders described herein, e.g., stroke, tumor, Parkinson's disease, sleep disturbances, side effects of medication, an infection, mild cognitive impairment, or a non-Alzheimer's dementia, including vascular dementia.

### III. Methods of Treatment

Disclosed herein are also methods of treating, controlling, ameliorating, or reducing a cognitive disorder (e.g., those described herein) in a subject in need thereof based on the diagnosis described herein. Accordingly, in some aspects, methods disclosed herein comprise administering a therapy to a subject identified as being afflicted with a cognitive disorder. In some aspects, the therapy is capable of treating, controlling, ameliorating, or reducing the cognitive disorder.

In some aspects, the therapy can comprise any agent (e.g., therapeutic agent) that can treat, control, ameliorate, or reduce one or more symptoms associated with a cognitive disorder disclosed herein. Non-limiting examples of symptoms associated with a cognitive disorder described herein include: memory loss, frequently asking the same question or repeating the same story over and over, difficulty recognizing familiar people and places, having trouble exercising judgment (e.g., knowing what to do in an emergency), change in mood or behavior, vision problems, difficulty planning and carrying out tasks (e.g., following a recipe or keeping track of monthly bills), and combinations thereof.

In some aspects, the therapy comprises a compound that inhibits miR-485-3p activity (“miR-485-3p inhibitor”). Additional disclosures relating to miR-485-3p inhibitors that can be used with the methods disclosed herein are provided elsewhere in the present disclosure (see, e.g., Section IV).

In some aspects, administering a miR-485-3p inhibitor to a subject (e.g., identified as having a cognitive disorder) decreases miR-485-3p activity in the subject by at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% or more, compared to a reference (e.g., miR-485-3p activity in a corresponding subject not treated with the miR-485-3p inhibitor).

In some aspects, administering a miR-485-3p inhibitor to a subject described herein decreases the expression and/or level of miR-485-3p in the subject by at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% or more, compared to a reference (e.g., miR-485-3p expression and/or level in a corresponding subject not treated with the miR-485-3p inhibitor).

In some aspects, the decreased activity and/or expression of miR-485-3p can reduce an amyloid beta (A3) plaque load in the subject identified as being afflicted with a cognitive disorder, compared to a reference (e.g., amyloid beta (A3) plaque load in the subject prior to the administering or amyloid beta (A3) plaque load in a corresponding subject not treated with the miR-485-3p inhibitor). As used herein, “amyloid beta plaque” refers to all forms of aberrant deposition of amyloid beta including large aggregates and small associations of a few amyloid beta peptides and can contain any variation of the amyloid beta peptides. Amyloid beta (A3) plaque is known to cause neuronal changes, e.g., aberrations in synapse composition, synapse shape, synapse density, loss of synaptic conductivity, changes in dendrite diameter, changes in dendrite length, changes in spine density, changes in spine area, changes in spine length, or changes in spine head diameter. In some aspects, administering a miR-485-3p inhibitor described herein reduces an amyloid beta plaque load in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 85%, at least about 90%, at least about 95%, or about 100% compared to a reference (e.g., subjects that did not receive an administration of the miR-485 inhibitor).

In some aspects, administering a miR-485-3p inhibitor to a subject (e.g., identified as having a cognitive disorder) reduces the occurrence or risk of occurrence of one or more symptoms of a cognitive disorder by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., corresponding subjects that did not receive an administration of the miR-485 inhibitor).

In some aspects, administering a miR-485-3p inhibitor to a subject (e.g., identified as having a cognitive disorder) reduces memory loss compared to a reference (e.g., memory loss in the subject prior to the administering or memory loss in a corresponding subject not treated with the miR-485-3p inhibitor). In some aspects, administering a miR-485-3p inhibitor reduces memory loss or the risk of occurrence of memory loss by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., corresponding subjects that did not receive an administration of the miR-485 inhibitor).

In some aspects, administering a miR-485-3p inhibitor to a subject (e.g., identified as having a cognitive disorder) improves memory retention compared to a reference (e.g., memory retention in the subject prior to the administering or memory retention in a corresponding subject that was not treated with the miR-485 inhibitor). In some aspects, administering a miR-485-3p inhibitor of the present disclosure improves and/or increases memory retention by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., corresponding subjects that did not receive an administration of the miR-485 inhibitor).

In some aspects, administering a miR-485-3p inhibitor to a subject (e.g., identified as having a cognitive disorder) improves spatial working memory compared to a reference (e.g., spatial working memory in the subject prior to the administering or spatial working memory in a corresponding subject that was not treated with the miR-485 inhibitor). As used herein, the term “spatial working memory” refers to the ability to keep spatial information activity in working memory over a short period of time. In some aspects, spatial working memory is improved and/or increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., corresponding subjects that did not receive an administration of the miR-485 inhibitor).

In some aspects, administering a miR-485-3p inhibitor to a subject (e.g., identified as having a cognitive disorder) increases the phagocytic activity of scavenger cells (e.g., glial cells) in the subject compared to a reference (e.g., phagocytic activity in the subject prior to the administering or phagocytic activity in a corresponding subject not treated with the miR-485-3p inhibitor). In some aspects, administering a miR-485-3p inhibitor increases dendritic spine density of a neuron in the subject (e.g., identified as having a cognitive disorder) by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., corresponding subjects that did not receive an administration of the miR-485 inhibitor).

In some aspects, administering a miR-485-3p inhibitor to a subject (e.g., identified as having a cognitive disorder) increases neurogenesis compared to a reference (e.g., neurogenesis in the subject prior to the administering or neurogenesis in a corresponding subject not treated with the miR-485 inhibitor). As used herein, the term “neurogenesis” refers to the process by which neurons are created. Neurogenesis encompasses proliferation of neural stem and progenitor cells, differentiation of these cells into new neural cell types, as well as migration and survival of the new cells. The term is intended to cover neurogenesis as it occurs during normal development, predominantly during pre-natal and peri-natal development, as well as neural cells regeneration that occurs following disease, damage or therapeutic intervention. Adult neurogenesis is also termed “nerve” or “neural” regeneration. In some aspects, administering a miR-485-3p inhibitor increases neurogenesis in the subject (e.g., identified as having a cognitive disorder) by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., corresponding subjects that did not receive an administration of the miR-485 inhibitor).

In some aspects, increasing and/or inducing neurogenesis is associated with increased proliferation, differentiation, migration, and/or survival of neural stem cells and/or progenitor cells. Accordingly, in some aspects, administering a miR-485-3p inhibitor to a subject (e.g., identified as having a cognitive disorder) can increase the proliferation of neural stem cells and/or progenitor cells in the subject. In certain aspects, the proliferation of neural stem cells and/or progenitor cells is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., corresponding subjects that did not receive an administration of the miR-485 inhibitor). In some aspects, the survival of neural stem cells and/or progenitor cells is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., corresponding subjects that did not receive an administration of the miR-485 inhibitor).

In some aspects, increasing and/or inducing neurogenesis is associated with an increased number of neural stem cells and/or progenitor cells. In certain aspects, the number of neural stem cells and/or progenitor cells is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., corresponding subjects that did not receive an administration of the miR-485 inhibitor).

In some aspects, increasing and/or inducing neurogenesis is associated with increased axon, dendrite, and/or synapse development. In certain aspects, axon, dendrite, and/or synapse development is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., corresponding subjects that did not receive an administration of the miR-485 inhibitor).

In some aspects, administering a miR-485-3p inhibitor to a subject (e.g., identified as having a cognitive disorder) prevents and/or inhibits the development of an amyloid beta plaque load in the subject. In some aspects, administering a miR-485-3p inhibitor to a subject (e.g., identified as having a cognitive disorder) delays the onset of the development of an amyloid beta plaque load in the subject. In some aspects, administering a miR-485-3p inhibitor to a subject (e.g., identified as having a cognitive disorder) lowers the risk of developing an amyloid beta plaque load.

In some aspects, administering a miR-485-3p inhibitor to a subject (e.g., identified as having a cognitive disorder) increases dendritic spine density of a neuron in the subject compared to a reference (e.g., dendritic spine density of a neuron in the subject prior to the administering or dendritic spine density of a neuron in a corresponding subject that was not treated with the miR-485-3p inhibitor). In some aspects, administering a miR-485-3p inhibitor increases dendritic spine density of a neuron in a subject (e.g., identified as having a cognitive disorder) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., corresponding subjects that did not receive an administration of the miR-485 inhibitor).

In some aspects, administering a miR-485-3p inhibitor to a subject (e.g., identified as having a cognitive disorder) decreases the loss of dendritic spines of a neuron in the subject compared to a reference (e.g., loss of dendritic spines of a neuron in the subject prior to the administering or loss of dendritic spines of a neuron in a corresponding subject that was not treated with the miR-485-3p inhibitor). In certain aspects, administering a miR-485-3p inhibitor decreases the loss of dendritic spines of a neuron in a subject (e.g., identified as having a cognitive disorder) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., corresponding subjects that did not receive an administration of the miR-485-3p inhibitor).

In some aspects, administering a miR-485-3p inhibitor to a subject (e.g., identified as having a cognitive disorder) decreases neuroinflammation in the subject compared to a reference (e.g., neuroinflammation in the subject prior to the administering or neuroinflammation in a corresponding subject that was not treated with the miR-485-3p inhibitor). In certain aspects, administering a miR-485-3p inhibitor decreases neuroinflammation by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., corresponding subjects that did not receive an administration of the miR-485-3p inhibitor). In some aspects, decreased neuroinflammation comprises glial cells producing decreased amounts of inflammatory mediators. Accordingly, in certain aspects, administering a miR-485-3p inhibitor to a subject (e.g., identified as having a cognitive disorder) decreases the amount of inflammatory mediators produced by glial cells by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., corresponding subjects that did not receive an administration of the miR-485 inhibitor). In some aspects, an inflammatory mediator produced by glial cells comprises TNF-α. In some aspects, the inflammatory mediator comprises IL-1β. In some aspects, an inflammatory mediator produced by glial cells comprises both TNF-α and IL-1β.

In some aspects, administering a miR-485-3p inhibitor to a subject (e.g., identified as having a cognitive disorder) increases autophagy in the subject. As used herein, the term “autophagy” refers to cellular stress response and a survival pathway that is responsible for the degradation of long-lived proteins, protein aggregates, as well as damaged organelles in order to maintain cellular homeostasis. In some aspects, administering a miR-485-3p inhibitor increases autophagy by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, or at least about 300% or more, compared to a reference (e.g., corresponding subjects that did not receive an administration of the miR-485-3p inhibitor).

In some aspects, administering a miR-485-3p inhibitor to a subject (e.g., identified as having a cognitive disorder) improves synaptic function in the subject compared to a reference (e.g., synaptic function in the subject prior to the administering). As used herein, the term “synaptic function,” refers to the ability of the synapse of a cell (e.g., a neuron) to pass an electrical or chemical signal to another cell (e.g., a neuron). In some aspects, administering a miR-485-3p inhibitor improves synaptic function in a subject (e.g., identified as having a cognitive disorder) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., corresponding subjects that did not receive an administration of the miR-485 inhibitor).

In some aspects, administering a miR-485-3p inhibitor to a subject (e.g., identified as having a cognitive disorder) can prevent, delay, and/or ameliorate the loss of synaptic function in the subject compared to a reference (e.g., loss of synaptic function in the subject prior to the administering or loss of synaptic function in a corresponding subject that was not treated with the miR-485-3p inhibitor). In some aspects, administering a miR-485-3p inhibitor prevents, delays, and/or ameliorates the loss of synaptic function in a subject (e.g., identified as having a cognitive disorder) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., corresponding

In some aspects, a miR-485-3p inhibitor disclosed herein can be administered by any suitable route known in the art. In certain aspects, a miR-485-3p inhibitor is administered parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intracistemally, intracapsularly, intratumorally, or any combination thereof.

In some aspects, a miR-485-3p inhibitor can be used in combination with one or more additional therapeutic agents. In some aspects, the additional therapeutic agent and the miR-485-3p inhibitor are administered concurrently. In certain aspects, the additional therapeutic agent and the miR-485-3p inhibitor are administered sequentially.

In some aspects, the administration of a miR-485-3p inhibitor disclosed herein does not result in any adverse effects. In certain aspects, miR-485-3p inhibitors of the present disclosure does not adversely affect body weight when administered to a subject. In some aspects, miR-485-3p inhibitors disclosed herein do not result in increased mortality or cause pathological abnormalities when administered to a subject.

### IV. miRNA-485-3p Inhibitors Useful for the Present Disclosure

Disclosed herein are compounds that can inhibit miR-485-3p activity (miR-485-3p inhibitor). In some aspects, a miR-485-3p inhibitor of the present disclosure comprises a nucleotide sequence encoding a nucleotide molecule that comprises at least one miR-485-3p binding site, wherein the nucleotide molecule does not encode a protein. As described herein, in some aspects, the miR-485-3p binding site is at least partially complementary to the target miRNA nucleic acid sequence (i.e., miR-485-3p), such that the miR-485-3p inhibitor hybridizes to the miR-485-3p nucleic acid sequence.

In some aspects, the miR-485-3p binding site of a miR-485-3p inhibitor disclosed herein has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence of a miR-485-3p. In certain aspects, the miR-485-3p binding site is fully complementary to the nucleic acid sequence of a miR-485-3p.

The miR-485-3p hairpin precursor can generate miR-485-3p. The human mature miR-485-3p has the sequence 5′-GUCAUACACGGCUCUCCUCUCU-3′ (SEQ ID NO: 1; miRBase Acc. No. MIMAT0002176). A 5′ terminal subsequence of miR-485-3p 5′-UCAUACA-3′ (SEQ ID NO: 49) is the seed sequence.

As will be apparent to those in the art, the human mature miR-485-3p has significant sequence similarity to that of other species. For instance, the mouse mature miR-485-3p differs from the human mature miR-485-3p by a single amino acid at each of the 5′- and 3′-ends (i.e., has an extra “A” at the 5′-end and missing “C” at the 3′-end). The mouse mature miR-485-3p has the following sequence: 5′-AGUCAUACACGGCUCUCCUCUC-3′ (SEQ ID NO: 34; miRBase Acc. No. MIMAT0003129; underlined portion corresponds to overlap to human mature miR-485-3p). Because of the similarity in sequences, in some aspects, a miR-485-3p inhibitor disclosed herein is capable of binding miR-485-3p from one or more species, e.g., human and mouse.

In some aspects, the miR-485-3p binding site is a single-stranded polynucleotide sequence that is complementary (e.g., fully complementary) to a sequence of a miR-485-3p (or a subsequence thereof). In some aspects, the miR-485-3p subsequence comprises the seed sequence. Accordingly, in certain aspects, the miR-485-3p binding site has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence set forth in SEQ ID NO: 49. In certain aspects, the miR-485-3p binding site is complementary to miR-485-3p except for 1, 2, 3, 4, 5, 6, 7, 8, 9, or 10 mismatches. In further aspects, the miR-485-3p binding site is fully complementary to the nucleic acid sequence set forth in SEQ ID NO: 1.

The seed region of a miRNA forms a tight duplex with the target mRNA. Most miRNAs imperfectly base-pair with the 3′ untranslated region (UTR) of target mRNAs, and the 5′ proximal “seed” region of miRNAs provides most of the pairing specificity. Without being bound to any theory, it is believed that the first nine miRNA nucleotides (encompassing the seed sequence) provide greater specificity whereas the miRNA ribonucleotides 3′ of this region allow for lower sequence specificity and thus tolerate a higher degree of mismatched base pairing, with positions 2-7 being the most important. Accordingly, in specific aspects of the present disclosure, the miR-485-3p binding site comprises a subsequence that is fully complementary (i.e., 100% complementary) over the entire length of the seed sequence of miR-485-3p.

miRNA sequences and miRNA binding sequences that can be used in the context of the disclosure include, but are not limited to, all or a portion of those sequences in the sequence listing provided herein, as well as the miRNA precursor sequence, or complement of one or more of these miRNAs. Any aspects of the disclosure involving specific miRNAs or miRNA binding sites by name is contemplated also to cover miRNAs or complementary sequences thereof whose sequences are at least about at least about 50%, at least about 55%, at least about 60%, at least about 65%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the mature sequence of the specified miRNA sequence or complementary sequence thereof.

In some aspects, miRNA binding sequences of the present disclosure can include additional nucleotides at the 5′, 3′, or both 5′ and 3′ ends of those sequences in the sequence listing provided herein, as long as the modified sequence is still capable of specifically binding to miR-485-3p. In some aspects, miRNA binding sequences of the present disclosure can differ in at least 1, 2, 3, 4, 5, 6, 7, 8, 9, 10 or more nucleotides with respect to those sequence in the sequence listing provided, as long as the modified sequence is still capable of specifically binding to miR-485-3p.

It is also specifically contemplated that any methods and compositions discussed herein with respect to miRNA binding molecules or miRNA can be implemented with respect to synthetic miRNAs binding molecules. It is also understood that the disclosures related to RNA sequences in the present disclosure are equally applicable to corresponding DNA sequences.

In some aspects, a miRNA-485 inhibitor of the present disclosure comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 5′ of the nucleotide sequence. In some aspects, a miRNA-485 inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 3′ of the nucleotide sequence.

In some aspects, a miR-485-3p inhibitor disclosed herein is about 6 to about 30 nucleotides in length. In certain aspects, a miR-485-3p inhibitor disclosed herein is 7 nucleotides in length. In further aspects, a miR-485-3p inhibitor disclosed herein is 8 nucleotides in length. In some aspects, a miR-485-3p inhibitor is 9 nucleotides in length. In some aspects, a miR-485-3p inhibitor of the present disclosure is 10 nucleotides in length. In certain aspects, a miR-485-3p inhibitor is 11 nucleotides in length. In further aspects, a miR-485-3p inhibitor is 12 nucleotides in length. In some aspects, a miR-485-3p inhibitor disclosed herein is 13 nucleotides in length. In certain aspects, a miR-485-3p inhibitor disclosed herein is 14 nucleotides in length. In some aspects, a miR-485-3p inhibitor disclosed herein is 15 nucleotides in length. In further aspects, a miR-485-3p inhibitor is 16 nucleotides in length. In certain aspects, a miR-485-3p inhibitor of the present disclosure is 17 nucleotides in length. In some aspects, a miR-485-3p inhibitor is 18 nucleotides in length. In some aspects, a miR-485-3p inhibitor is 19 nucleotides in length. In certain aspects, a miR-485-3p inhibitor is 20 nucleotides in length. In further aspects, a miR-485-3p inhibitor of the present disclosure is 21 nucleotides in length. In some aspects, a miR-485-3p inhibitor is 22 nucleotides in length.

In some aspects, a miR-485-3p inhibitor disclosed herein comprises a nucleotide sequence that is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence selected from SEQ ID NOs: 2 to 30. In certain aspects, a miR-485-3p inhibitor comprises a nucleotide sequence selected from the group consisting of SEQ ID NOs: 2 to 30, wherein the nucleotide sequence can optionally comprise 1, 2, 3, 4, 5, 6, 7, 8, 9, or 10 mismatches.

In some aspects, a miRNA inhibitor comprises 5′-UGUAUGA-3′ (SEQ ID NO: 2), 5′-GUGUAUGA-3′ (SEQ ID NO: 3), 5′-CGUGUAUGA-3′ (SEQ ID NO: 4), 5′-CCGUGUAUGA-3′ (SEQ ID NO: 5), 5′-GCCGUGUAUGA-3′ (SEQ ID NO: 6), 5′-AGCCGUGUAUGA-3′ (SEQ ID NO: 7), 5′-GAGCCGUGUAUGA-3′ (SEQ ID NO: 8), 5′-AGAGCCGUGUAUGA-3′ (SEQ ID NO: 9), 5′-GAGAGCCGUGUAUGA-3′ (SEQ ID NO: 10), 5′-GGAGAGCCGUGUAUGA-3′ (SEQ ID NO: 11), 5′-AGGAGAGCCGUGUAUGA-3′ (SEQ ID NO: 12), 5′-GAGGAGAGCCGUGUAUGA-3′ (SEQ ID NO: 13), 5′-AGAGGAGAGCCGUGUAUGA-3′ (SEQ ID NO: 14), or 5′-GAGAGGAGAGCCGUGUAUGA-3′ (SEQ ID NO: 15).

In some aspects, the miRNA inhibitor has 5′-UGUAUGAC-3′ (SEQ ID NO: 16), 5′-GUGUAUGAC-3′ (SEQ ID NO: 17), 5′-CGUGUAUGAC-3′ (SEQ ID NO: 18), 5′-CCGUGUAUGAC-3′ (SEQ ID NO: 19), 5′-GCCGUGUAUGAC-3′ (SEQ ID NO: 20), 5′-AGCCGUGUAUGAC-3′ (SEQ ID NO: 21), 5′-GAGCCGUGUAUGAC-3′ (SEQ ID NO: 22), 5′-AGAGCCGUGUAUGAC-3′ (SEQ ID NO: 23), 5′-GAGAGCCGUGUAUGAC-3′ (SEQ ID NO: 24), 5′-GGAGAGCCGUGUAUGAC-3′ (SEQ ID NO: 25), 5′-AGGAGAGCCGUGUAUGAC-3′ (SEQ ID NO: 26), 5′-GAGGAGAGCCGUGUAUGAC-3′ (SEQ ID NO: 27), 5′-AGAGGAGAGCCGUGUAUGAC-3′ (SEQ ID NO: 28), 5′-GAGAGGAGAGCCGUGUAUGAC-3′ (SEQ ID NO: 29), or 5′-AGAGAGGAGAGCCGUGUAUGAC-3′ (SEQ ID NO: 30).

In some aspects, the miRNA inhibitor has a sequence selected from the group consisting of: 5′-TGTATGA-3′ (SEQ ID NO: 62), 5′-GTGTATGA-3′ (SEQ ID NO: 63), 5′-CGTGTATGA-3′ (SEQ ID NO: 64), 5′-CCGTGTATGA-3′ (SEQ ID NO: 65), 5′-GCCGTGTATGA-3′ (SEQ ID NO: 66), 5′-AGCCGTGTATGA-3′ (SEQ ID NO: 67), 5′-GAGCCGTGTATGA-3′ (SEQ ID NO: 68), 5′-AGAGCCGTGTATGA-3′ (SEQ ID NO: 69), 5′-GAGAGCCGTGTATGA-3′ (SEQ ID NO: 70), 5′-GGAGAGCCGTGTATGA-3′ (SEQ ID NO: 71), 5′-AGGAGAGCCGTGTATGA-3′ (SEQ ID NO: 72), 5′-GAGGAGAGCCGTGTATGA-3′ (SEQ ID NO: 73), 5′-AGAGGAGAGCCGTGTATGA-3′ (SEQ ID NO: 74), 5′-GAGAGGAGAGCCGTGTATGA-3′ (SEQ ID NO: 75); 5′-TGTATGAC-3′ (SEQ ID NO: 76), 5′-GTGTATGAC-3′ (SEQ ID NO: 77), 5′-CGTGTATGAC-3′ (SEQ ID NO: 78), 5′-CCGTGTATGAC-3′ (SEQ ID NO: 79), 5′-GCCGTGTATGAC-3′ (SEQ ID NO: 80), 5′-AGCCGTGTATGAC-3′ (SEQ ID NO: 81), 5′-GAGCCGTGTATGAC-3′ (SEQ ID NO: 82), 5′-AGAGCCGTGTATGAC-3′ (SEQ ID NO: 83), 5′-GAGAGCCGTGTATGAC-3′ (SEQ ID NO: 84), 5′-GGAGAGCCGTGTATGAC-3′ (SEQ ID NO: 85), 5′-AGGAGAGCCGTGTATGAC-3′ (SEQ ID NO: 86), 5′-GAGGAGAGCCGTGTATGAC-3′ (SEQ ID NO: 87), 5′-AGAGGAGAGCCGTGTATGAC-3′ (SEQ ID NO: 88), 5′-GAGAGGAGAGCCGTGTATGAC-3′ (SEQ ID NO: 89); and 5′-AGAGAGGAGAGCCGTGTATGAC-3′ (SEQ ID NO: 90).

In some aspects, a miRNA inhibitor disclosed herein (i.e., miR-485-3p inhibitor) comprises a nucleotide sequence that is at least about 50%, at least about 55%, at least about 60%, at least about 65%, at least about 70%, at least about 75%, at least about 80%, at least about 85%, at least about 90%, or at least about 95% identical to 5′-AGAGGAGAGCCGUGUAUGAC-3′ (SEQ ID NO: 28) or 5′-AGAGGAGAGCCGTGTATGAC-3′ (SEQ ID NO: 88). In some aspects, the miRNA inhibitor comprises a nucleotide sequence that has at least 90% similarity to 5′-AGAGGAGAGCCGUGUAUGAC-3′ (SEQ ID NO: 28) or 5′-AGAGGAGAGCCGTGTATGAC-3′ (SEQ ID NO: 88). In some aspects, the miRNA inhibitor comprises the nucleotide sequence 5′-AGAGGAGAGCCGUGUAUGAC-3′ (SEQ ID NO: 28) or 5′-AGAGGAGAGCCGTGTATGAC-3′ (SEQ ID NO: 88) with one substitution or two substitutions. In certain aspects, the miRNA inhibitor comprises the nucleotide sequence 5′-AGAGGAGAGCCGUGUAUGAC-3′ (SEQ ID NO: 28) or 5′-AGAGGAGAGCCGTGTATGAC-3′ (SEQ ID NO: 88).

In some aspects, the sequence of miR-485-3p inhibitor is at least about 50%, at least about 55%, at least about 60%, at least about 65%, at least about 70%, at least about 75%, at least about 80%, at least about 85%, at least about 90%, or at least about 95% sequence identity to 5′-AGAGAGGAGAGCCGUGUAUGAC-3′ (SEQ ID NO: 30) or 5′-AGAGAGGAGAGCCGTGTATGAC-3′ (SEQ ID NO: 90). In certain aspects, the miRNA inhibitor has a sequence that has at least 90% similarity to 5′-AGAGAGGAGAGCCGUGUAUGAC-3′ (SEQ ID NO: 30) or 5′-AGAGAGGAGAGCCGTGTATGAC-3′ (SEQ ID NO: 90). In some aspects, the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90) with one substitution or two substitutions. In some aspects, the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90). In some aspects, the miRNA inhibitor comprises the nucleotide sequence 5′-AGAGAGGAGAGCCGUGUAUGAC-3′ (SEQ ID NO: 30).

In some aspects, a miR-485-3p inhibitor of the present disclosure comprises the sequence disclosed herein, e.g., any one of SEQ ID NOs: 2 to 30, and at least one, at least two, at least three, at least four or at least five additional nucleic acid at the N terminus, at least one, at least two, at least three, at least four, or at least five additional nucleic acid at the C terminus, or both. In some aspects, a miR-485-3p inhibitor of the present disclosure comprises the sequence disclosed herein, e.g., any one of SEQ ID NOs: 2 to 30, and one additional nucleic acid at the N terminus and/or one additional nucleic acid at the C terminus. In some aspects, a miR-485-3p inhibitor of the present disclosure comprises the sequence disclosed herein, e.g., any one of SEQ ID NOs: 2 to 30, and one or two additional nucleic acids at the N terminus and/or one or two additional nucleic acids at the C terminus. In some aspects, a miR-485-3p inhibitor of the present disclosure comprises the sequence disclosed herein, e.g., any one of SEQ ID NOs: 2 to 30, and one to three additional nucleic acids at the N terminus and/or one to three additional nucleic acids at the C terminus. In some aspects, a miR-485-3p inhibitor comprises 5′-GAGAGGAGAGCCGUGUAUGAC-3′ (SEQ ID NO: 29). In certain aspects, a miR-485 inhibitor comprises 5′-AGAGAGGAGAGCCGUGUAUGAC-3′ (SEQ ID NO: 30).

In some aspects, a miR-485-3p inhibitor of the present disclosure comprises one miR-485-3p binding site. In further aspects, a miR-485-3p inhibitor disclosed herein comprises at least two miR-485-3p binding sites. In certain aspects, a miR-485-3p inhibitor comprises three miR-485-3p binding sites. In some aspects, a miR-485-3p inhibitor comprises four miR-485-3p binding sites. In some aspects, a miR-485-3p inhibitor comprises five miR-485-3p binding sites. In certain aspects, a miR-485-3p inhibitor comprises six or more miR-485-3p binding sites. In some aspects, all the miR-485-3p binding sites are identical. In some aspects, all the miR-485-3p binding sites are different. In some aspects, at least one of the miR-485-3p binding sites is different.

IV.a. Chemically Modified Polynucleotides

In some aspects, a miR-485-3p inhibitor disclosed herein comprises a polynucleotide which includes at least one chemically modified nucleoside and/or nucleotide. When the polynucleotides of the present disclosure are chemically modified the polynucleotides can be referred to as “modified polynucleotides.”

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can be synthesized by any useful method, such as, for example, chemically, enzymatically, or recombinantly, to include one or more modified or non-natural nucleosides.

Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In some aspects, the modified polynucleotides contain one, two, or more (optionally different) nucleoside or nucleotide modifications. In some aspects, a modified polynucleotide can exhibit one or more desirable properties, e.g., improved thermal or chemical stability, reduced immunogenicity, reduced degradation, increased binding to the target microRNA, reduced non-specific binding to other microRNA or other molecules, as compared to an unmodified polynucleotide.

In some aspects, a polynucleotide of the present disclosure (e.g., a miR-485-3p inhibitor) is chemically modified. As used herein, in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof.

In some aspects, a polynucleotide of the present disclosure (e.g., a miR-485-3p inhibitor) can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation In further aspects, the polynucleotide of the present disclosure (e.g., a miR-485-3p inhibitor) can have a uniform chemical modification of two, three, or four of the same nucleoside type throughout the entire polynucleotide (such as all uridines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s. For example, TD's of the present disclosure can be administered as RNAs, as DNAs, or as hybrid molecules comprising both RNA and DNA units.

In some aspects, the polynucleotide (e.g., a miR-485-3p inhibitor) includes a combination of at least two (e.g., 2, 3, 4, 5, 6, 7, 8, 8, 10, 11, 12, 13, 14, 15, 16, 17, 18, 18, 20 or more) modified nucleobases.

In some aspects, the nucleobases, sugar, backbone linkages, or any combination thereof in a polynucleotide are modified by at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100%.

(i) Base Modification

In certain aspects, the chemical modification is at nucleobases in a polynucleotide of the present disclosure (e.g., a miR-485-3p inhibitor). In some aspects, the at least one chemically modified nucleoside is a modified uridine (e.g., pseudouridine (ψ), 2-thiouridine (s2U), 1-methyl-pseudouridine (m1ψ), 1-ethyl-pseudouridine (e1ψ), or 5-methoxy-uridine (mo5U)), a modified cytosine (e.g., 5-methyl-cytidine (m5C)) a modified adenosine (e.g, 1-methyl-adenosine (m1A), N6-methyl-adenosine (m6A), or 2-methyl-adenine (m2A)), a modified guanosine (e.g., 7-methyl-guanosine (m7G) or 1-methyl-guanosine (m1G)), or a combination thereof.

In some aspects, the polynucleotide of the present disclosure (e.g., a miR-485-3p inhibitor) is uniformly modified (e.g., fully modified, modified throughout the entire sequence) for a particular modification. For example, a polynucleotide can be uniformly modified with the same type of base modification, e.g., 5-methyl-cytidine (m5C), meaning that all cytosine residues in the polynucleotide sequence are replaced with 5-methyl-cytidine (m5C). Similarly, a polynucleotide can be uniformly modified for any type of nucleoside residue present in the sequence by replacement with a modified nucleoside such as any of those set forth above.

In some aspects, the polynucleotide of the present disclosure (e.g., a miR-485-3p inhibitor) includes a combination of at least two (e.g., 2, 3, 4 or more) of modified nucleobases. In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of a type of nucleobases in a polynucleotide of the present disclosure (e.g., a miR-485-3p inhibitor) are modified nucleobases.

(ii) Backbone Modifications

In some aspects, the polynucleotide of the present disclosure (i.e., miR-485-3p inhibitor) can include any useful linkage between the nucleosides. Such linkages, including backbone modifications, that are useful in the composition of the present disclosure include, but are not limited to the following: 3-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

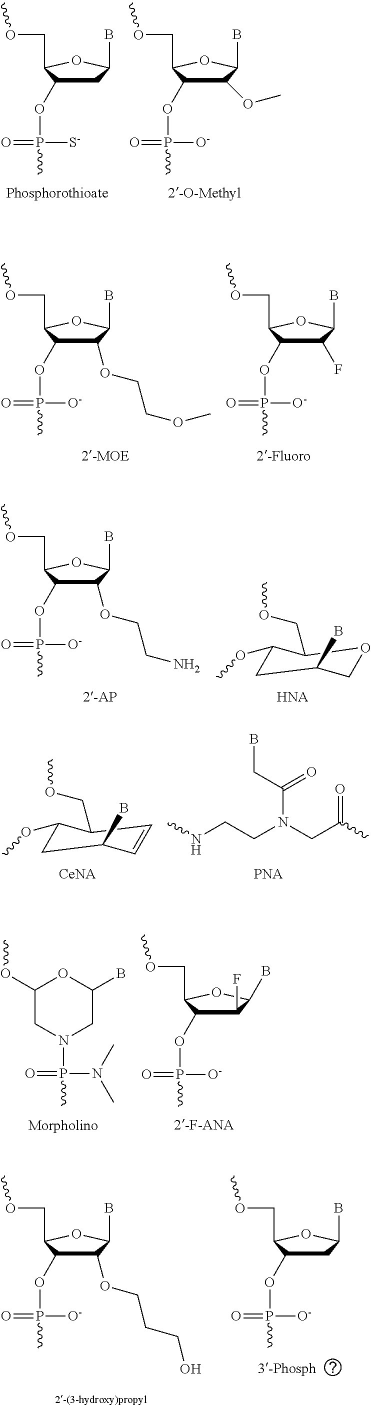

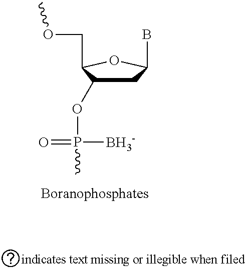

In some aspects, the presence of a backbone linkage disclosed above increase the stability and resistance to degradation of a polynucleotide of the present disclosure (i.e., miR-485-3p inhibitor).

In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the backbone linkages in a polynucleotide of the present disclosure (i.e., miR-485-3p inhibitor) are modified (e.g., all of them are phosphorothioate).

In some aspects, a backbone modification that can be included in a polynucleotide of the present disclosure (i.e., miR-485-3p inhibitor) comprises phosphorodiamidate morpholino oligomer (PMO) and/or phosphorothioate (PS) modification.

(iii) Sugar Modifications

The modified nucleosides and nucleotides which can be incorporated into a polynucleotide of the present disclosure (i.e., miR-485-3p inhibitor) can be modified on the sugar of the nucleic acid. In some aspects, the sugar modification increases the affinity of the binding of a miR-485-3p inhibitor to miR-485-3p nucleic acid sequence. Incorporating affinity-enhancing nucleotide analogues in the miR-485-3p inhibitor, such as LNA or 2′-substituted sugars, can allow the length and/or the size of the miR-485-3p inhibitor to be reduced.

In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the nucleotides in a polynucleotide of the present disclosure (i.e., miR-485-3p inhibitor) contain sugar modifications (e.g., LNA).

In some aspects, 1, 2, 3, 4, 5, 6, 7, 8, 9, 10, 11, 12, 13, 14, 15, 16, 17, 18, 19, 20, 21, or 22 nucleotide units in a polynucleotide of the present disclosure are sugar modified (e.g., LNA).

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The 2′ hydroxyl group (OH) of ribose can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈cycloalkyl; optionally substituted C₃₋₈cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, amino bridges, aminoalkyl, aminoalkoxy, amino, and amino acid.

In some aspects, nucleotide analogues present in a polynucleotide of the present disclosure (i.e., miR-485-3p inhibitor) comprise, e.g., 2′-O-alkyl-RNA units, 2′-OMe-RNA units, 2′-O-alkyl-SNA, 2′-amino-DNA units, 2′-fluoro-DNA units, LNA units, arabino nucleic acid (ANA) units, 2′-fluoro-ANA units, HNA units, INA (intercalating nucleic acid) units, 2′MOE units, or any combination thereof. In some aspects, the LNA is, e.g., oxy-LNA (such as beta-D-oxy-LNA, or alpha-L-oxy-LNA), amino-LNA (such as beta-D-amino-LNA or alpha-L-amino-LNA), thio-LNA (such as beta-D-thio-LNA or alpha-L-thio-LNA), ENA (such a beta-D-ENA or alpha-L-ENA), or any combination thereof. In further aspects, nucleotide analogues that can be included in a polynucleotide of the present disclosure (i.e., miR-485-3p inhibitor) comprises a locked nucleic acid (LNA), an unlocked nucleic acid (UNA), an arabino nucleic acid (ABA), a bridged nucleic acid (BNA), and/or a peptide nucleic acid (PNA).

In some aspects, a polynucleotide of the present disclosure (i.e., miR-485-3p inhibitor) can comprise both modified RNA nucleotide analogues (e.g., LNA) and DNA units. In some aspects, a miR-485-3p inhibitor is a gapmer. See, e.g., U.S. Pat. Nos. 8,404,649; 8,580,756; 8,163,708; 9,034,837; all of which are herein incorporated by reference in their entireties. In some aspects, a miR-485-3p inhibitor is a micromir. See U.S. Pat. Appl. Publ. No. US20180201928, which is herein incorporated by reference in its entirety.

In some aspects, a polynucleotide of the present disclosure (i.e., miR-485-3p inhibitor) can include modifications to prevent rapid degradation by endo- and exo-nucleases. Modifications include, but are not limited to, for example, (a) end modifications, e.g., 5′ end modifications (phosphorylation, dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

### V. Vectors and Delivery Systems

In some aspects, the miR-485-3p inhibitors disclosed herein can be administered (e.g., identified as being afflicted with a cognitive disorder) using any relevant delivery system known in the art. In certain aspects, the delivery system is a vector. Accordingly, in some aspects, the present disclosure provides a vector comprising a miR-485-3p inhibitor of the present disclosure.

In some aspects, the vector is viral vector. In some aspects, the viral vector is an adenoviral vector or an adeno-associated viral vector. In certain aspects, the viral vector is an AAV that has a serotype of AAV2, AAV3, AAV4, AAV5, AAV6, AAV7, AAV8, AAV9, AAV10, or any combination thereof. In some aspects, the adenoviral vector is a third generation adenoviral vector. ADEASY™ is by far the most popular method for cTeating adenoviral vector constructs. The system consists of two types of plasmids: shuttle (or transfer) vectors and adenoviral vectors. The transgene of interest is cloned into the shuttle vector, verified, and linearized with the restriction enzyme PmeI. This construct is then transformed into ADEASIER-1 cells, which are BJ5183 E. coli cells containing PADEASY™ PADEASY™ is a ˜33 Kb adenoviral plasmnid containing the adenoviral genes necessary for virus production. The shuttle vector and the adenoviral plasmid have matching left and right homology arms which facilitate homologous recombination of the transgene into the adenoviral plasmid. One can also co-transform standard BJ5183 with supercoiled PADEASY™ and the shuttle vector, but this method results in a higher background of non-recombinant adenoviral plasmids. Recombinant adenoviral plasmids are then verified for size and proper restriction digest patterns to determine that the transgene has been inserted into the adenoviral plasmid, and that other patterns of recombination have not occurred. Once verified, the recombinant plasmid is linearized with Pac1 to create a linear dsDNA construct flanked by ITRs. 293 or 911 cells are transfected with the linearized construct, and virus can be harvested about 7-10 days later. In addition to this method, other methods for creating adenoviral vector constructs known in the art at the time the present application was filed can be used to practice the methods disclosed herein.

In some aspects, the viral vector is a retroviral vector, e.g., a lentiviral vector (e.g., a third or fourth generation lentiviral vector). Lentiviral vectors are usually created in a transient transfection system in which a cell line is transfected with three separate plasmid expression systems. These include the transfer vector plasmid (portions of the HIV provirus), the packaging plasmid or construct, and a plasmid with the heterologous envelop gene (env) of a different virus. The three plasmid components of the vector are put into a packaging cell which is then inserted into the HIV shell. The virus portions of the vector contain insert sequences so that the virus cannot replicate inside the cell system. Current third generation lentiviral vectors encode only three of the nine HIV-1 proteins (Gag, Pol, Rev), which are expressed from separate plasmids to avoid recombination-mediated generation of a replication-competent virus. In fourth generation lentiviral vectors, the retroviral genome has been further reduced (see, e.g., TAKARA® LENTI-X™ fourth-generation packaging systems).

Any AAV vector known in the art can be used in the methods disclosed herein. The AAV vector can comprise a known vector or can comprise a variant, fragment, or fusion thereof. In some aspects, the AAV vector is selected from the group consisting of AAV type 1 (AAV1), AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, bovine AAV, shrimp AVV, snake AVV, and any combination thereof.

In some aspects, the AAV vector is derived from an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In some aspects, the AAV vector is a chimeric vector derived from at least two AAV vectors selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In certain aspects, the AAV vector comprises regions of at least two different AAV vectors known in the art.

In some aspects, the AAV vector comprises an inverted terminal repeat from a first AAV (e.g., AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, or any derivative thereof) and a second inverted terminal repeat from a second AAV (e.g., AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, or any derivative thereof).

In some aspects, the AVV vector comprises a portion of an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof. In some aspects, the AAV vector comprises AAV2.

In some aspects, the AVV vector comprises a splice acceptor site. In some aspects, the AVV vector comprises a promoter. Any promoter known in the art can be used in the AAV vector of the present disclosure. In some aspects, the promoter is an RNA Pol III promoter. In some aspects, the RNA Pol III promoter is selected from the group consisting of the U6 promoter, the H1 promoter, the 7SK promoter, the 5S promoter, the adenovirus 2 (Ad2) VAI promoter, and any combination thereof. In some aspects, the promoter is a cytomegalovirus immediate-early gene (CMV) promoter, an EF1a promoter, an SV40 promoter, a PGK1 promoter, a Ubc promoter, a human beta actin promoter, a CAG promoter, a TRE promoter, a UAS promoter, a Ac5 promoter, a polyhedrin promoter, a CaMKIIa promoter, a GAL1 promoter, a GAL10 promoter, a TEF promoter, a GDS promoter, a ADH1 promoter, a CaMV35S promoter, or a Ubi promoter. In a specific aspect, the promoter comprises the U6 promoter.

In some aspects, the AAV vector comprises a constitutively active promoter (constitutive promoter). In some aspects, the constitutive promoter is selected from the group consisting of hypoxanthine phosphoribosyl transferase (HPRT), adenosine deaminase, pyruvate kinase, beta-actin promoter, cytomegalovirus (CMV), simian virus (e.g., SV40), papilloma virus, adenovirus, human immunodeficiency virus (HIV), Rous sarcoma virus, a retrovirus long terminal repeat (LTR), Murine stem cell virus (MSCV) and the thymidine kinase promoter of herpes simplex virus.

In some aspects, the promoter is an inducible promoter. In some aspects, the inducible promoter is a tissue specific promoter. In certain aspects, the tissue specific promoter drives transcription of the coding region of the AVV vector in a neuron, a glial cell, or in both a neuron and a glial cell.

In some aspects, the AVV vector comprises one or more enhancers. In some aspects, the one or more enhancer are present in the AAV alone or together with a promoter disclosed herein. In some aspects, the AAV vector comprises a 3′UTR poly(A) tail sequence. In some aspects, the 3′UTR poly(A) tail sequence is selected from the group consisting of bGH poly(A), actin poly(A), hemoglobin poly(A), and any combination thereof. In some aspects, the 3′UTR poly(A) tail sequence comprises bGH poly(A).

In some aspects, a miR-485-3p inhibitor disclosed herein is administered with a delivery agent. Non-limiting examples of delivery agents that can be used include a lipidoid, a liposome, a lipoplex, a lipid nanoparticle, a polymeric compound, a peptide, a protein, a cell, a nanoparticle mimic, a nanotube, a micelle, or a conjugate.

Thus, in some aspects, the present disclosure also provides a composition comprising a miRNA inhibitor of the present disclosure (i.e., miR-485-3p inhibitor) and a delivery agent. In some aspects, the delivery agent comprises a cationic carrier unit comprising

[WP]-L1-[CC]-L2-[AM] (formula I)

or

[WP]-L1-[AM]-L2-[CC] (formula II)

wherein
WP is a water-soluble biopolymer moiety;
CC is a positively charged (i.e., cationic) carrier moiety;
AM is an adjuvant moiety; and,
L1 and L2 are independently optional linkers, and
wherein when mixed with a nucleic acid at an ionic ratio of about 1:1, the cationic carrier unit forms a micelle. Accordingly, in some aspects, the miRNA inhibitor and the cationic carrier unit are capable of associating with each other (e.g., via a covalent bond or a non-valent bond) to form a micelle when mixed together.

In some aspects, composition comprising a miRNA inhibitor of the present disclosure (i.e., miR-485-3p inhibitor) interacts with the cationic carrier unit via an ionic bond.

In some aspects, the water-soluble polymer comprises poly(alkylene glycols), poly(oxyethylated polyol), poly(oletinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof. In some aspects, the water-soluble polymer comprises polyethylene glycol (“PEG”), polyglycerol, or poly(propylene glycol) (“PPG”). In some aspects, the water-soluble polymer comprises:

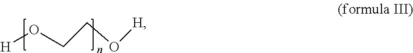

wherein n is 1-1000.

In some aspects, the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In some aspects, the n is about 80 to about 90, about 90 to about 100, about 100 to about 110, about 110 to about 120, about 120 to about 130, about 140 to about 150, about 150 to about 160.

In some aspects, the water-soluble polymer is linear, branched, or dendritic. In some aspects, the cationic carrier moiety comprises one or more basic amino acids. In some aspects, the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids. In some aspects, the cationic carrier moiety comprises about 30 to about 50 basic amino acids. In some aspects, the basic amino acid comprises arginine, lysine, histidine, or any combination thereof. In some aspects, the cationic carrier moiety comprises about 40 lysine monomers.

In some aspects, the adjuvant moiety is capable of modulating an immune response, an inflammatory response, and/or a tissue microenvironment. In some aspects, the adjuvant moiety comprises an imidazole derivative, an amino acid, a vitamin, or any combination thereof. In some aspects, the adjuvant moiety comprises:

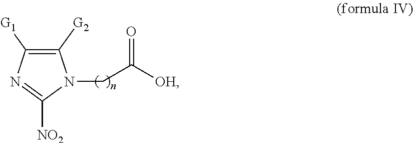

wherein each of G1 and G2 is H, an aromatic ring, or 1-10 alkyl, or G1 and G2 together form an aromatic ring, and wherein n is 1-10.

In some aspects, the adjuvant moiety comprises nitroimidazole. In some aspects, the adjuvant moiety comprises metronidazole, tinidazole, nimorazole, dimetridazole, pretomanid, ornidazole, megazol, azanidazole, benznidazole, or any combination thereof. In some aspects, the adjuvant moiety comprises an amino acid.

In some aspects, the adjuvant moiety comprises

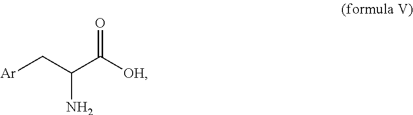

wherein Ar is

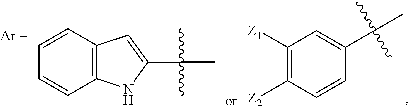

and
wherein each of Z1 and Z2 is H or OH.

In some aspects, the adjuvant moiety comprises a vitamin. In some aspects, the vitamin comprises a cyclic ring or cyclic hetero atom ring and a carboxyl group or hydroxyl group. In some aspects, the vitamin comprises:

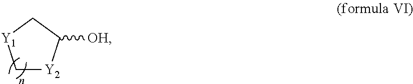

wherein each of Y1 and Y2 is C, N, O, or S, and wherein n is 1 or 2.

In some aspects, the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof. In some aspects, the vitamin is vitamin B3.

In some aspects, the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3. In some aspects, the adjuvant moiety comprises about 10 vitamin B3.

In some aspects, the composition comprises a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

In some aspects, the composition comprises (i) a water-soluble biopolymer moiety with about 100 to about 200 PEG units, (ii) about 30 to about 40 lysines with an amine group (e.g., about 32 lysines), (iii) about 15 to 20 lysines, each having a thiol group (e.g., about 16 lysines, each with a thiol group), and (iv) about 30 to 40 lysines fused to vitamin B3 (e.g., about 32 lysines, each fused to vitamin B3). In some aspects, the composition further comprises a targeting moiety, e.g., a LAT1 targeting ligand, e.g., phenyl alanine, linked to the water soluble polymer. In some aspects, the thiol groups in the composition form disulfide bonds.

In some aspects, the composition comprises (1) a micelle comprising (i) about 100 to about 200 PEG units, (ii) about 30 to about 40 lysines with an amine group (e.g., about 32 lysines), (iii) about 15 to 20 lysines, each having a thiol group (e.g., about 16 lysines, each with a thiol group), and (iv) about 30 to 40 lysines fused to vitamin B3 (e.g., about 32 lysines, each fused to vitamin B3), and (2) a miR485 inhibitor (e.g., SEQ ID NO: 30), wherein the miR485 inhibitor is encapsulated within the micelle. In some aspects, the composition further comprises a targeting moiety, e.g., a LAT1 targeting ligand, e.g., phenyl alanine, linked to the PEG units. In some aspects, the thiol groups in the micelle form disulfide bonds.

The present disclosure also provides a micelle comprising a miRNA inhibitor of the present disclosure (i.e., miR-485-3p inhibitor) wherein the miRNA inhibitor and the delivery agent are associated with each other.

In some aspects, the association is a covalent bond, a non-covalent bond, or an ionic bond. In some aspects, the positive charge of the cationic carrier moiety of the cationic carrier unit is sufficient to form a micelle when mixed with the miR-485-3p inhibitor disclosed herein in a solution, wherein the overall ionic ratio of the positive charges of the cationic carrier moiety of the cationic carrier unit and the negative charges of the miR-485-3p inhibitor (or vector comprising the inhibitor) in the solution is about 1:1.

In some aspects, the cationic carrier unit is capable of protecting the miRNA inhibitor of the present disclosure (i.e., miR-485-3p inhibitor) from enzymatic degradation. See U.S. PCT Publication No. WO2020/261227, which is herein incorporated by reference in its entirety.

### VI. Pharmaceutical Compositions

In some aspects, the present disclosure also provides pharmaceutical compositions comprising a miR-485-3p inhibitor disclosed herein (e.g., a polynucleotide or a vector comprising the miR-485-3p inhibitor) that are suitable for administration to a subject. The pharmaceutical compositions generally comprise a miR-485-3p inhibitor described herein (e.g., a polynucleotide or a vector) and a pharmaceutically-acceptable excipient or carrier in a form suitable for administration to a subject. Pharmaceutically acceptable excipients or carriers are determined in part by the particular composition being administered, as well as by the particular method used to administer the composition.

Accordingly, there is a wide variety of suitable formulations of pharmaceutical compositions comprising a miR-485-3p inhibitor of the present disclosure. (See, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration.

### VII. Kits

The present disclosure also provides kits or products of manufacture, comprising a miRNA inhibitor of the present disclosure (e.g., a polynucleotide, vector, or pharmaceutical composition disclosed herein) and optionally instructions for use, e.g., instructions for use according to the methods disclosed herein. In some aspects, the kit or product of manufacture comprises a miR-485-3p inhibitor (e.g., vector, e.g., an AAV vector, a polynucleotide, or a pharmaceutical composition of the present disclosure) in one or more containers. In some aspects, the kit or product of manufacture comprises miR-485-3p inhibitor (e.g., a vector, e.g., an AAV vector, a polynucleotide, or a pharmaceutical composition of the present disclosure) and a brochure. One skilled in the art will readily recognize that miR-485-3p inhibitors disclosed herein (e.g., vectors, polynucleotides, and pharmaceutical compositions of the present disclosure, or combinations thereof) can be readily incorporated into one of the established kit formats which are well known in the art.

The following examples are offered by way of illustration and not by way of limitation.

### EXAMPLES

### Example 1: Materials and Methods

The Examples described below use one or more of the following materials and methods.

### Clinical Sample and Information

All human-derived samples were from patients recruited based in International Review Board (IRB) approved at Konyang University Hospital, Boramae Medical Center, Eulji Medical Center, and Gyeongsang National University Hospital in South Korea. In total, 21 amyloid PET negative patients and 26 amyloid PET positive patients were recruited. Table 3A (below) provides clinical information for one or more of these patients.

TABLE 3A
Clinical Information for Recruited Patients

| | | | | | | ³Amyloid | | | Education | ⁶APOE |
|---|---|---|---|---|---|---|---|---|---|---|
| ¹Site | Tissue Type | ID | Age | Gender | ²DX | PET result | ⁴MMSE | ⁵CDR | year | genotype |
| Site1 | Swab | Site1-1 | 66 | Male | NC | Negative | 27 | 0 | 16 | E3/E4 |
| Site1 | Swab | Site1-2 | 80 | Male | NC | Negative | 23 | 0 | 16 | E3/E3 |
| Site1 | Swab | Site1-3 | 73 | Female | NC | Negative | 27 | 0 | 6 | E3/E3 |
| Site1 | Swab | Site1-4 | 78 | Female | NC | Negative | 26 | 0 | 0 | E2/E3 |
| Site1 | Swab | Site1-5 | 68 | Female | NC | Negative | 26 | 0 | 0 | E2/E3 |
| Site1 | Swab | Site1-6 | 73 | Female | NC | Negative | 25 | 0 | 6 | E3/E4 |
| Site1 | Swab | Site1-7 | 76 | Female | NC | Negative | 27 | 0 | 10 | E3/E3 |
| Site1 | Swab | Site1-8 | 81 | Female | NC | Negative | 23 | 0 | 7 | E3/E3 |
| Site1 | Swab | Site1-9 | 69 | Female | MCI | Negative | 25 | 0.5 | 2 | E3/E3 |
| Site1 | Swab | Site1-10 | 58 | Male | NC | Positive | 29 | 0 | 16 | E3/E3 |
| Site1 | Swab | Site1-11 | 78 | Male | MCI | Positive | 26 | 0.5 | 12 | E3/E3 |
| Site1 | Swab | Site1-12 | 86 | Female | MCI | Positive | 22 | 0.5 | 7 | E3/E3 |
| Site1 | Swab | Site1-13 | 70 | Female | MCI | Positive | 22 | 0.5 | 7 | E3/E4 |
| Site1 | Swab | Site1-14 | 76 | Male | MCI | Positive | 22 | 0.5 | 0 | E3/E4 |
| Site1 | Swab | Site1-15 | 65 | Male | MCI | Positive | 30 | 0.5 | 12 | E3/E4 |
| Site1 | Swab | Site1-16 | 70 | Female | MCI | Positive | 24 | 0.5 | 6 | E4/E4 |
| Site1 | Swab | Site1-17 | 85 | Male | AD | Positive | 18 | 0.5 | 6 | E3/E3 |
| Site1 | Swab | Site1-18 | 74 | Female | AD | Positive | 17 | 0.5 | 4 | E3/E3 |
| Site1 | Swab | Site1-19 | 85 | Female | AD | Positive | 16 | 1 | 6 | E2/E4 |
| Site1 | Swab | Site1-20 | 73 | Female | AD | Positive | 24 | 0.5 | 16 | E4/E4 |
| Site2 | Swab, Plasma | Site2-1 | 75 | Female | NC | Negative | 29 | 0 | 9 | E3/E3 |
| Site2 | Swab, Plasma | Site2-2 | 81 | Female | NC | Negative | 23 | 0 | 6 | E3/E3 |
| Site2 | Swab, Plasma | Site2-3 | 89 | Female | NC | Negative | 28 | 0 | 6 | E2/E3 |
| Site2 | Swab, Plasma | Site2-4 | 75 | Female | NC | Negative | 27 | 0 | 9 | E2/E3 |
| Site2 | Swab, Plasma | Site2-5 | 74 | Male | AD | Positive | 23 | 0.5 | 6 | E4/E4 |
| Site3 | Swab | Site3-1 | 60 | Female | NC | Negative | 30 | 0.5 | 15 | E2/E4 |
| Site3 | Swab | Site3-2 | 58 | Female | NC | Negative | 27 | 0.5 | 5 | E3/E4 |
| Site3 | Swab, Plasma | Site3-3 | 66 | Female | NC | Negative | 27 | 0.5 | 6 | E2/E3 |
| Site3 | Swab, Plasma | Site3-4 | 67 | Male | MCI | Negative | 26 | 0.5 | 6 | E3/E3 |
| Site3 | Swab, Plasma | Site3-5 | 72 | Female | MCI | Negative | 25 | 0.5 | 6 | E3/E3 |
| Site3 | Swab | Site3-6 | 57 | Male | NC | Positive | 29 | 0.5 | 16 | E3/E3 |
| Site3 | Swab, Plasma | Site3-7 | 66 | Female | NC | Positive | 27 | 0 | 9 | E3/E3 |
| Site3 | Swab, Plasma | Site3-8 | 70 | Male | MCI | Positive | 27 | 0.5 | 16 | E3/E4 |
| Site3 | Swab, Plasma | Site3-9 | 71 | Male | MCI | Positive | 26 | 0.5 | 18 | E3/E4 |
| Site3 | Swab, Plasma | Site3-10 | 64 | Male | MCI | Positive | 27 | 0.5 | 12 | E3/E3 |
| Site3 | Swab, Plasma | Site3-11 | 80 | Male | AD | Positive | 20 | 0.5 | 6 | E3/E3 |
| Site3 | Swab | Site3-12 | 61 | Male | AD | Positive | 20 | 0.5 | 12 | E3/E3 |
| Site3 | Swab, Plasma | Site3-13 | 78 | Male | AD | Positive | 23 | 1 | 12 | E3/E4 |
| Site3 | Swab | Site3-14 | 53 | Male | AD | Positive | 16 | 1 | 12 | E3/E4 |
| Site3 | Swab | Site3-15 | 68 | Female | AD | Positive | 27 | 1 | 6 | E4/E4 |
| Site3 | Swab, Plasma | Site3-16 | 71 | Female | AD | Positive | 16 | 0.5 | 1 | E3/E3 |
| Site4 | Plasma | Site4-1 | 59 | Female | NC | Negative | | | | |
| Site4 | Plasma | Site4-2 | 61 | Male | NC | Negative | | | | |
| Site4 | Plasma | Site4-3 | 64 | Female | NC | Negative | | | | |
| Site4 | Plasma | Site4-4 | 84 | Female | AD | Positive | | | | |
| Site4 | Plasma | Site4-5 | 79 | Female | AD | Positive | | | | |
| Site4 | Plasma | Site4-6 | 88 | Male | AD | Positive | | | | |

Clinical information of 47 patients.
¹Site means the hospital where the sample was taken. Site1 is Seoul National University Boramae Medical Center. Site2 is Eulji Medical Center. Site3 is Gyeongsang National University Hospital.
²DX was a diagnosis result using cognitive function and various clinical results by a clinical specialist.
³Amyloid PET results were the results of Amyloid PET CT imaging, as determined by a nuclear medicine specialist. Negative means the result with little or no accumulation of amyloid beta, and positive means the result with amyloid beta accumulation.
⁴MMSE was an abbreviation for mini mental state examination.
⁵CDR was an abbreviation for clinical dementia rate.
⁶APOE genotype was a result derived from genotyping of the innate allele type of APOE gene.

For the real-time PCR assay experiments, three amyloid PET negative patients and eight amyloid PET positive patients were recruited. Table 3B (below) provides the clinical information for the samples obtained from these patients.

TABLE 3B
Clinical samples and Information used in Real-time PCR experiments

| ¹Site | ID | Age | Gender | ²DX | ³PET | ⁴MMSE | ⁵CDR | ^{e}EDU | ⁷APOE |
|---|---|---|---|---|---|---|---|---|---|
| EMC | EMC016 | 75 | Female | NC | Negative | 29 | 0 | 9 | E3/E3 |
| EMC | EMC019 | 74 | Male | AD | Positive | 23 | 0.5 | 6 | E4/E4 |
| EMC | EMC020 | 81 | Female | NC | Negative | 23 | 0 | 6 | E3/E3 |
| EMC | EMC021 | 89 | Female | NC | Negative | 28 | 0 | 6 | E2/E3 |
| EMC | EMC022 | 82 | Male | AD | Negative | 21 | 0.5 | 9 | E3/E4 |
| EMC | EMC023 | 75 | Female | NC | Negative | 27 | 0 | 9 | E2/E3 |
| GNUH | GNUH001 | 60 | Female | NC | Negative | 30 | 0.5 | 15 | E2/E4 |
| GNUH | GNUH002 | 70 | Male | MCI | Positive | 27 | 0.5 | 16 | E3/E4 |
| GNUH | GNUH003 | 67 | Male | MCI | Negative | 26 | 0.5 | 6 | E3/E3 |
| GNUH | GNUH004 | 72 | Female | MCI | Negative | 25 | 0.5 | 6 | E3/E3 |
| GNUH | GNUH005 | 68 | Female | AD | Positive | 27 | 1 | 6 | E4/E4 |
| GNUH | GNUH007 | 71 | Male | MCI | Positive | 26 | 0.5 | 18 | E3/E4 |
| GNUH | GNUH008 | 57 | Male | NC | Positive | 29 | 0.5 | 16 | E3/E3 |
| GNUH | GNUH010 | 71 | Female | AD | Positive | 16 | 0.5 | 1 | E3/E3 |
| GNUH | GNUH011 | 58 | Female | NC | Negative | 27 | 0.5 | 5 | E3/E4 |
| GNUH | GNUH012 | 66 | Female | NC | Negative | 27 | 0.5 | 6 | E2/E3 |
| GNUH | GNUH013 | 66 | Female | NC | Positive | 27 | 0 | 9 | E3/E3 |
| GNUH | GNUH014 | 64 | Male | MCI | Positive | 27 | 0.5 | 12 | E3/E3 |
| GNUH | GNUH015 | 80 | Male | AD | Positive | 20 | 0.5 | 6 | E3/E3 |
| GNUH | GNUH016 | 61 | Male | AD | Positive | 20 | 0.5 | 12 | E3/E3 |
| GNUH | GNUH017 | 78 | Male | AD | Positive | 23 | 1 | 12 | E3/E4 |
| GNUH | GNUH018 | 53 | Male | AD | Positive | 16 | 1 | 12 | E3/E4 |

¹SITE: Institution or hospital from which the sample was obtained. “EMC” is Eulji Medical Center. “GNUH” is Gyeongsang National University Hospital;
²DX: Results of specialist diagnosis for (i) normal cognitive (NC), (ii) mild cognitive impairment (MCI), or (iii) Alzheimer's disease (AD);
³PET: After amyloid-β PET (Positron Emission Tomography) CT (Computed Tomography) imaging, diagnosed by nuclear medicine specialists and related specialists;
⁴MMSE: mini mental state examination score;
⁵CDR: clinical dementia rating;
⁶EDU: education year;
⁷APOE: APOE genotype.

### Diagnosis of Alzheimer's Disease and Amyloid-β PET CT Imaging

Amyloid-β was measured using amyloid-β PET CT imaging at a medical institution. Using the imaging results, amyloid-β PET positive and negative judgment was made by nuclear medicine specialists and neurologists. Patients with amyloid-β accumulation were classified as “amyloid PET positive.” Otherwise, patients were classified as “amyloid PET negative.” Alzheimer's disease diagnosis was carried out by a medical specialist. Diagnosis was divided into one of the following categories: (i) Normal Cognitive (NC), (ii) Mild Cognitive Impairment (MCI), and (iii) Alzheimer's Disease (AD).

### Patient's Oral Epithelial Cell Collection (Swab Samples)

To collect the oral epithelial cells, a single cotton swab was used to wipe the inside of a patient's mouth (about 5-10 times). From each patient, a total of 10 different swab samples were collected. Each of the swab samples were collected in a separate e-tube. Then, the tubes were labeled with the patient ID and stored at −20° C. until further analysis.

### Oligonucleotides for PCR Amplification

As described herein, miR-485-3p expression was quantified using real-time PCR. The different primers and probes used are provided in Table 4 (below).

TABLE 4
Primers and probes for PCR amplification of miR-485-3p

| Oligonucleotide | | PCR |
|---|---|---|
| Name | Sequence | Method |
| miR-485-3p_FW1 | 5′-GTCATACACGGCTCTCCTCTCT-3′ (SEQ ID NO: 94) | Real-time |
| miR-485-3p_FW7 | 5′-GTCATACACGGCTCTCCTCTCTAA-3′ (SEQ ID NO: 100) | Real-time |
| miR-485-3p_FW9 | 5′-CATACACGGCTCTCCTCTCTAAA-3′ (SEQ ID NO: 52) | Real-time |
| Reverse | 5′-GAATCGAGCACCAGTTACG-3′ (SEQ ID NO: 60) | Real-time |
| Fluorescent probe | FAM-CGAGGTCGACTTCCTAGA-NFQ (SEQ ID NO: 108) | Real-time |

### Micro-RNA Preparation

The micro-RNAs were extracted using miRNeasy serum/plasma Kit (Qiagen, Germany) according to the manufacturer's instructions. Micro-RNAs from exosomes of oral epithelium and plasma were extracted using exoRNeasy Serum/Plasma Midi Kits (Qiagen, Germany) according to the manufacturer's instructions. Then, 1 μg of the extracted micro-RNA was used for cDNA synthesis using miScript II RT Kit (Qiagen, Hilden, Germany).

### Preparation of Standard Materials

miR-485-3p's mimic (sequence: GUCAUACACGGCUCUCCUCUCU (SEQ ID NO: 1); human microRNA sequence from miRDB; mirdb.org/index.html) was synthesized by ssDNA using miScript II RT kit (Qiagen, Cat. 218161). The concentration of the synthesized ssDNA was measured using a Quantus™ Fluorometer (Promega, E6150) instrument. Thereafter, ssDNA was purified using MEGAquick-spin™ Plus Total Fragment DNA Purification Kit (Intron, 17290).

### Real-Time PCR

To analyze miRNA expression, TaqMan miRNA analysis was performed using TOPreal™ qPCR 2× PreMIX (Enzynomics, Korea) on CFX connect system (Bio-Rad). The real-time PCR measurement of individual cDNAs was performed using TaqMan probe to measure duplex DNA formation with the Bio-Rad real-time PCR system. Real-time PCR was performed by diluting the cDNA samples to varying concentrations. Then, 10 ng/μL of cDNA were used for general micro-RNA preparation, and 2 ng/μL of cDNA were used for exosomal micro-RNA preparation. Real-time PCR was performed as follows: 95° C. for 15 min, 40 cycles (95° C. for 10 s, 55° C. for 1 min, 72° C. for 10 s). The experiments were carried out in duplicate for each data point. Data were exported from BioRad software and imported into analysis program.

Quantification of microRNA

Real-time PCR results using the miR-485-3p primers described herein (see Table 4 above) were output as cycle threshold (Ct) value. Then, the Ct value was substituted with the expression value determined using the following formula: Expression value=2^{-cycle threshold}×10¹⁰.

Next, the standard material was pre-weighed in the following amounts: (1) 0.01 pg; (2) 0.03 pg; (3) 0.05 pg; (4) 0.07 pg; (5) 0.09 pg; and (6) 0.20 pg. The Ct value for the standard material was also substituted with the expression value determined using the formula provided above. The regression equation was obtained for each batch using the pre-measured quantity of the standard material and the associated expression value. To quantify the miRNA expression level, the patient's miR-485-3p expression value (described earlier) was plugged into the above obtained regression equation.

### APOE Genotyping

APOE genotyping was performed as described in Zhong et al., Mol Neurodegener 11:2-4 (2016), which is incorporated herein by reference in its entirety. Briefly, approximately 1 mL of DPBS was added to tubes containing the oral clinical swab samples, and vortexed. Then, the cotton swabs were removed, and the tube centrifuged at 13,000 rpm for three minutes. The supernatant was discarded to prepare a precipitate. Next, the genomic DNA was extracted from the precipitate using a Higene genomic DNA prep kit (Biofact, GD141-100). The primers, probes, and qPCR mix were prepared as provided in Tables 5 and 6 (below). Mixtures of E2, E3, and E4 were prepared and 1 μL of the extracted genomic DNA was added to the mixture. Then, real-time PCR amplification was performed using BioRad CFX 96.

TABLE 5
PCR reaction mixture for APOE genotyping

| PCR reaction mix | Volume (ul) |
|---|---|
| 2× master mix(2 QPP) | 10 |
| E2/E3/E4 primer(10p/F + R) | 2 |
| ApoE probe(10p) | 1 |
| Template (10-30 ng) | 1 |
| Rnase-free water | 6 |
| Total mixture | 20 |

TABLE 6
Primer information for APOE genotyping

| Name | Sequence (5′-3′) | nt |
|---|---|---|
| E2-Forward | GCGGACATGGAGGACGTGT | 19 |
| | (SEQ ID NO: 109) | |
| E2-Reverse | CCTGGTACACTGCCAGGCA | 19 |
| | (SEQ ID NO: 110) | |
| E3-Forward | CGGACATGGAGGACGTGT | 18 |
| | (SEQ ID NO: 111) | |
| E3-Reverse | CTGGTACACTGCCAGGCG | 18 |
| | (SEQ ID NO: 112) | |
| E4-Forward | CGGACATGGAGGACGTGC | 18 |
| | (SEQ ID NO: 113) | |
| E4-Reverse | CTGGTACACTGCCAGGCG | 18 |
| | (SEQ ID NO: 112) | |
| ApoE Probe | [FAM] CAGCTCCTCGGTGCTCTGGC [BHQ1] | 20 |
| | (SEQ ID NO: 114) | |

### Western Blotting for Verification of Exosomal RNA

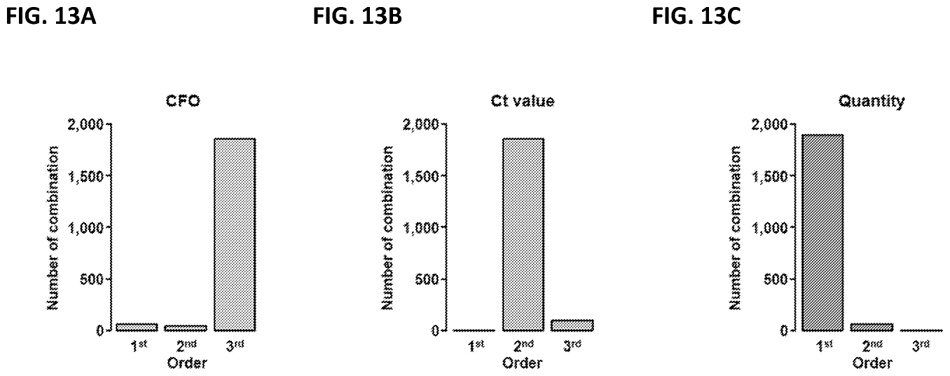

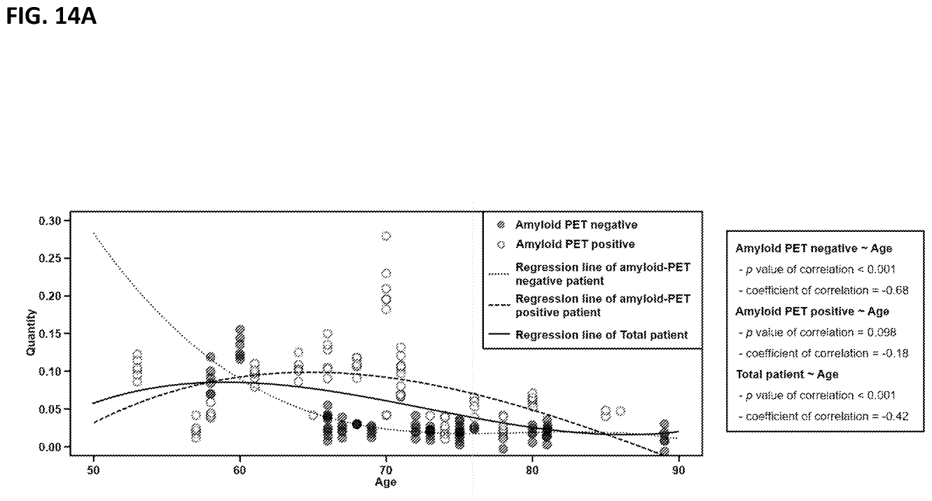

The tubes containing the oral clinical swab samples were processed as described herein to obtain pellets and supernatant. The supernatant was used for further extraction of exosomes using exoRNeasy Serum/Plasma Midi kit (Qiagen, Cat. 77144). The cell pellet and HOCFE were fractionated by SDS-PAGE and transferred to a polyvinylidene difluoride membrane using a transfer apparatus according to the manufacturer's protocols. The purity of the fractionation can be seen in FIG. 13A. After incubation with 10% nonfat milk in TBST (10 mM Tris, pH 8.0, 150 mM NaCl, 0.5% Tween 20) for 60 min, the membrane was washed once with TBST and incubated with antibodies against CD81 (1:100) and Calnexin (1:200) at 4° C. for 12 h. Membranes were washed three times for 10 min and incubated with a 1:1000 dilution of IgG light chain binding protein conjugated to horseradish peroxidase for 1 h. Blots were washed with TBST three times and developed with the ECL system (Amersham Biosciences) according to the manufacturer's protocols.

### Preparation of Amyloid-β and Cell Treatment

A3 (amyloid-beta) 1-42 hexafluoroisopropanal (HFIP) peptide (#AS-64129) was obtained from AnaSpec (Fremont, Calif., USA). To form the amyloid-β monomer, HFIP peptide was dissolved in DMSO to a stock concentration of 5 mM. Stocks were then diluted to 100 μM in serum free DMEM. To form the amyloid-β oligomer, the monomers were incubated at 4° C. for 24 hours.

Human primary oral epithelial cells (Cat #36063-01) were purchased from Celprogen (Torrance, Calif.). The cells (5×10⁵ cells/well) were plated onto 6-well plates overnight. Cells were then treated with varying concentrations (0.1, 0.5, 1 μM) of the amyloid-β monomer or oligomer, and allowed to incubate for 6 hours. After the incubation, both the supernatant and cells were harvested for analysis.

### Prediction Model Construction Using Clinical Information

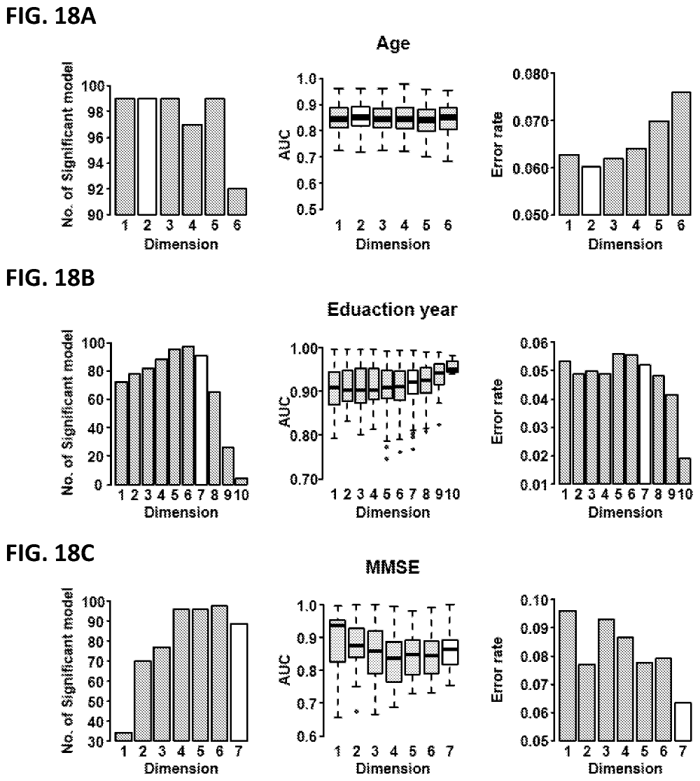

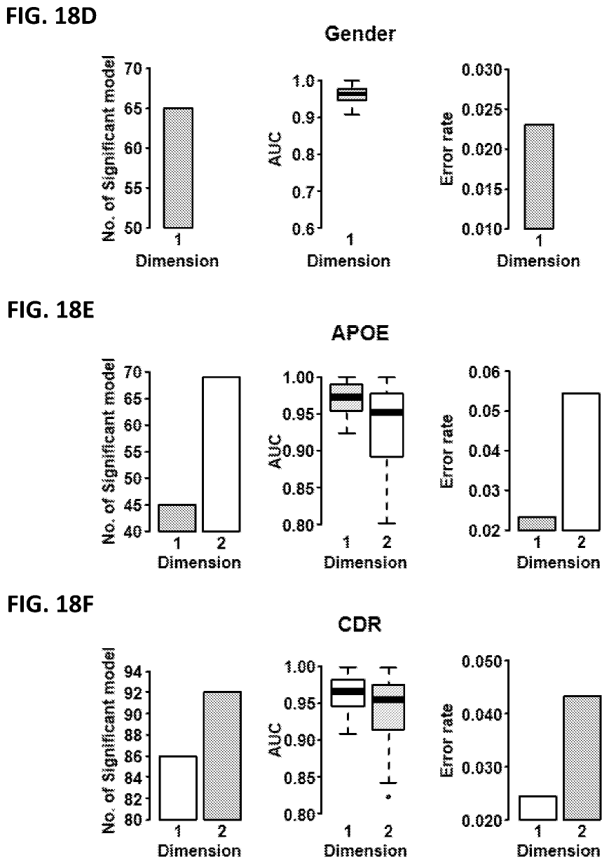

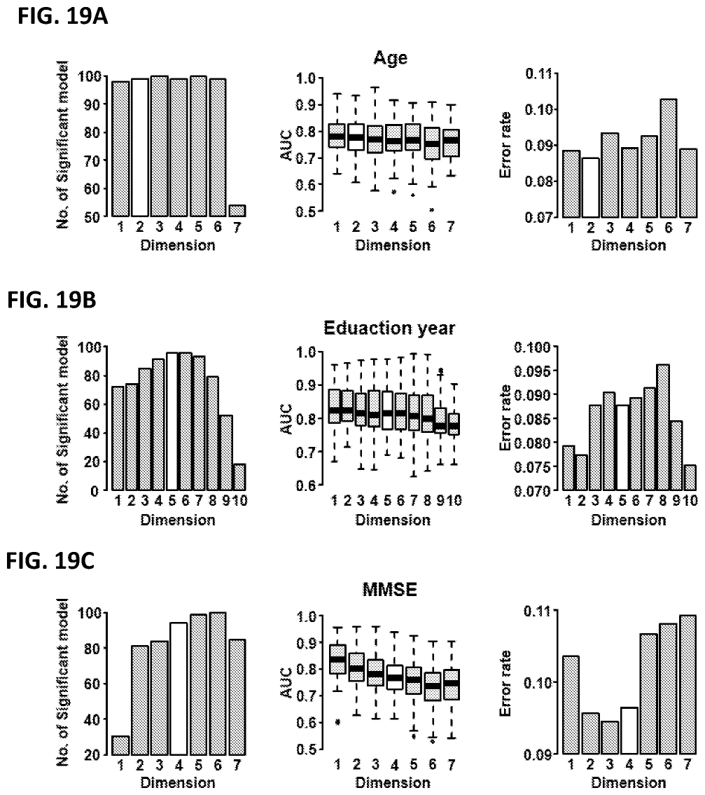

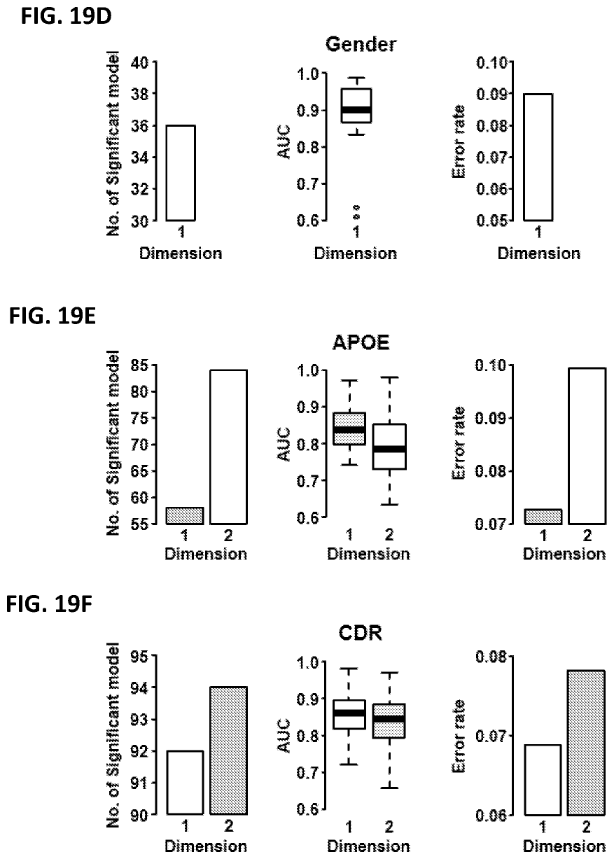

To calculate the relevant values described herein, various clinical information were assigned a value: (i) gender (male=1; female=2); and (ii) APOE genotype (E2/E3=1; E3/E3=1; E2/E4=2; E3/E4=2; and E4/E4=4). Age, educational year (0 to 18), MMSE score and CDR were used as they were. In order to determine the dimension with the highest AUC and lowest error rate, simulation was repeated 100 times using random sampling up to 11^{th} dimensions for each clinical information. The dimension was applied until all coefficients of the model were normally output. Accordingly, the number of dimensions for which a value could be determined varied among the different clinical information (see FIGS. 18A-18F and 19A-19F). After random sampling, the number of tests with a p value of ANOVA test of 0.05 or less was counted for each test. Once the optimal dimension was determined, clinical information was used to construct the algorithm with the quantity value of miR-485-3p. Clinical information was applied to the number of cases that do not overlap in any order.

In order to assess how each clinical information affects the change in accuracy, the following formula was used to calculate the “accuracy correction rate”: Accuracy correction rate (%)=[(ACC1/ACC2)−1]×100, wherein “ACC1” refers to the accuracy of algorithms containing specific clinical information, and “ACC2” refers to the algorithm accuracy without specific clinical information.

### Cross Validation and Computational Random Sampling

To validate and/or modify the algorithms disclosed herein, a K-fold cross validation method was used. In patient group with 41 subjects, the group was divided into three groups with 10 patients and one group with 11 patients. In patient group with 36 subjects (>60 years in age), the group was divided into four groups with nine patients. Models were created by merging the three groups and validated with the remaining one group. The same process was performed 4 times while replacing the validation set. Random sampling was performed by repeating 20 non-overlapping samples 100 times at random from all samples.

### Calculation of Gray Zone

The first cut-off value was derived as the simulated result value by dividing the value between the maximum value and the minimum value of the quantity or score value into 500 values. The most accurate cut-off value of the simulation results were the first cut-off value. Then, the mean of the standard deviation across all samples was calculated and divided in half. The gray zone was determined by adding and subtracting the first cut-off value to the half of the mean of the standard deviation calculated earlier. Upper of gray zone=1^{st} cut-off value+half of mean of SD for sample's quantity or score. Lower of gray zone=1^{st} cut-off value−half of mean of SD for sample's quantity or score

### Statistical Test

All statistical analyses were done through R (version 3.5.2). The statistical significance test between the two groups was performed by unpaired t-test. The ROC (Receiver Operating Characteristic) analysis was used to measure the AUC (area under curve) value, sensitivity and specificity. ROC analysis was performed using “ROCR” and “pROC”, a type of R package. The regression modeling was analyzed using R's “lm” command.

### Example 2: Selection of Target MicroRNAs

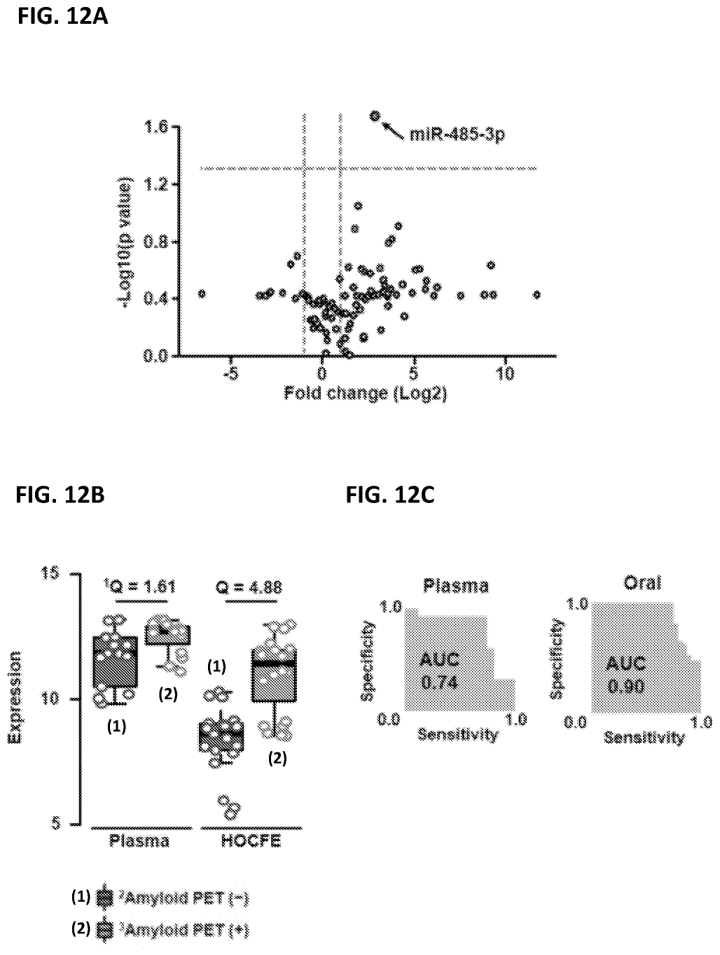

To identify specific miRNAs that could be used in diagnosing a cognitive disorder disclosed herein (e.g., Alzheimer's disease), the expression of different miRNAs was determined using qPCR in plasma samples from patients diagnosed with Alzheimer's disease (AD) and normal control subjects (i.e., normal cognitive). As shown in FIG. 12A, there was a statistically significant increased expression of miR-485-3p in plasma samples from the AD patients compared to the corresponding expression in plasma samples from the control subjects. Of the miRNAs tested, no other miRNAs exhibited such significant difference in expression.

To confirm the results described above, both plasma samples and oral clinical swab samples were collected from additional patients. Again, as shown in FIGS. 12B and 12C, miR-485-3p expression was significantly higher in the plasma samples from AD patients (i.e., amyloid PET positive) compared to the control subjects (i.e., amyloid PET negative). The difference in miR-385-3p expression was even more dramatic in the human oral-derived cell free exosomes, which were prepared from the oral clinical swab samples.

The above results demonstrate the increased miR-485-3p expression in patients with certain cognitive disorders (e.g., Alzheimer's disease), suggesting that miR-485-3p is a suitable biomarker candidate.

### Example 3: Analysis of Potential Bias in Clinical Information

To assess whether patient's clinical information can suggest any potential bias as to amyloid-β accumulation in a patient, the following clinical information were gathered and assigned a value where appropriate: (i) age at diagnosis, (ii) gender (male=1; female=2), (iii) education year (0-16), (iv) APOE genotype (E2/E3=1; E3/E3=1; E2/E4=2; E3/E4=2; and E4/E4=4), (v) mini mental state examination (MMSE) score; (vi) cognitive impairment (i.e., normal cognitive (“NC”); mild cognitive impairment (“MCI); and Alzheimer's disease (“AD”)), and (vii) clinical dementia rating (CDR) score. Then, each of the clinical information was assessed in patients confirmed to have amyloid-β accumulation (i.e., amyloid PET positive) and patients confirmed to not have amyloid-β accumulation (i.e., amyloid PET negative).

As shown in Table 7 (below), among patient groups diagnosed as having normal cognitive (NC) or with a CDR value of 0, there were significantly less amyloid PET positive subjects compared to amyloid PET negative subject. There were no statistically significant difference for the other clinical information between amyloid PET positive and negative subjects.

TABLE 7
Statistical Results for Clinical Information

| | | Number of | Number of | | | |
|---|---|---|---|---|---|---|
| | | amyloid | amyloid | | | 95% |
| Clinical | | PET | PET | | Odd | confidence |
| information | Value | negative | positive | p value | ratio | interval |
| Diagnosis by | NC | 15 | 3 | 0.005 | 0.17 | 0.03-0.61 |
| cognitive | | | | | | |
| impairment | MCI | 3 | 9 | 0.239 | 2.25 | 0.56-11.95 |
| Diagnosis by | | | | | | |
| cognitive | | | | | | |
| impairment | AD | 0 | 11 | — | — | — |
| Diagnosis by | | | | | | |
| cognitive | | | | | | |
| impairment | 0 | 12 | 2 | 0.007 | 0.14 | 0.02-0.62 |
| CDR value | | | | | | |
| CDR value | 0.5 | 6 | 17 | 0.158 | 2.17 | 0.72-7.18 |
| CDR value | 1 | 0 | 4 | — | — | — |
| Gender | Male | 3 | 14 | 0.058 | 3.47 | 0.93-17.65 |
| Gender | Female | 15 | 9 | 0.148 | 0.48 | 0.16-1.34 |
| Age at the | 51-60 | 2 | 3 | 0.868 | 1.15 | 0.16-10.73 |
| diagnosis | | | | | | |
| Age at the | 61-70 | 5 | 8 | 0.730 | 1.24 | 0.34-4.85 |
| diagnosis | | | | | | |
| Age at the | 71-80 | 8 | 9 | 0.826 | 0.88 | 0.28-2.84 |
| diagnosis | | | | | | |
| Age at the | 81-90 | 3 | 3 | 0.779 | 0.79 | 0.12-5.07 |
| diagnosis | | | | | | |
| Education year | 0 | 2 | 1 | 0.445 | 0.42 | 0.01-5.61 |
| Education year | 1-6 | 9 | 8 | 0.530 | 0.70 | 0.22-2.23 |
| Education year | 7-9 | 3 | 3 | 0.779 | 0.79 | 0.12-5.07 |
| Education year | 10-12 | 1 | 6 | 0.139 | 4.14 | 0.60-114.33 |
| Education year | 13-18 | 3 | 5 | 0.738 | 1.28 | 0.26-7.37 |
| MMSE score | 15-19 | 0 | 5 | — | — | — |
| MMSE score | 20-25 | 6 | 9 | 0.794 | 1.16 | 0.35-4.14 |
| MMSE score | 26-30 | 12 | 9 | 0.323 | 0.59 | 0.20-1.73 |
| APOE genotype | E2/E2 | 0 | 0 | — | — | — |
| APOE genotype | E2/E3 | 5 | 0 | — | — | — |
| APOE genotype | E3/E3 | 9 | 11 | 0.935 | 0.96 | 0.32-2.89 |
| APOE genotype | E2/E4 | 1 | 1 | 0.865 | 0.79 | 0.02-32.14 |
| APOE genotype | E3/E4 | 3 | 7 | 0.423 | 1.76 | 0.41-9.66 |
| APOE genotype | E4/E4 | 0 | 4 | — | — | — |

The above results suggest that the use of clinical information alone is not an effective predictor of cognitive disorders, such as those associated with amyloid-β accumulation (e.g., Alzheimer's disease).

### Example 4: Real-Time PCR Analysis of miR-485-3p Expression in Human Clinical Swab Samples

To begin assessing the use of miR-485-3p expression as a diagnostic marker for cognitive disorders (e.g., Alzheimer's disease), a real-time PCR assay was used to compare miR-485-3p expression in human clinical swab samples from patients with or without amyloid-βaccumulation. As described herein, amyloid-β accumulation is associated with many cognitive disorders. To amplify the miR-485-3p present in the clinical swab samples, the following primer was used: 5′-GTCATACACGGCTCTCCTCTCTAA-3′ (referred to herein as “miR-485-3p_FW7”; SEQ ID NO: 100). As shown above, the RNAs prepared for real time PCR were exosomal RNAs.

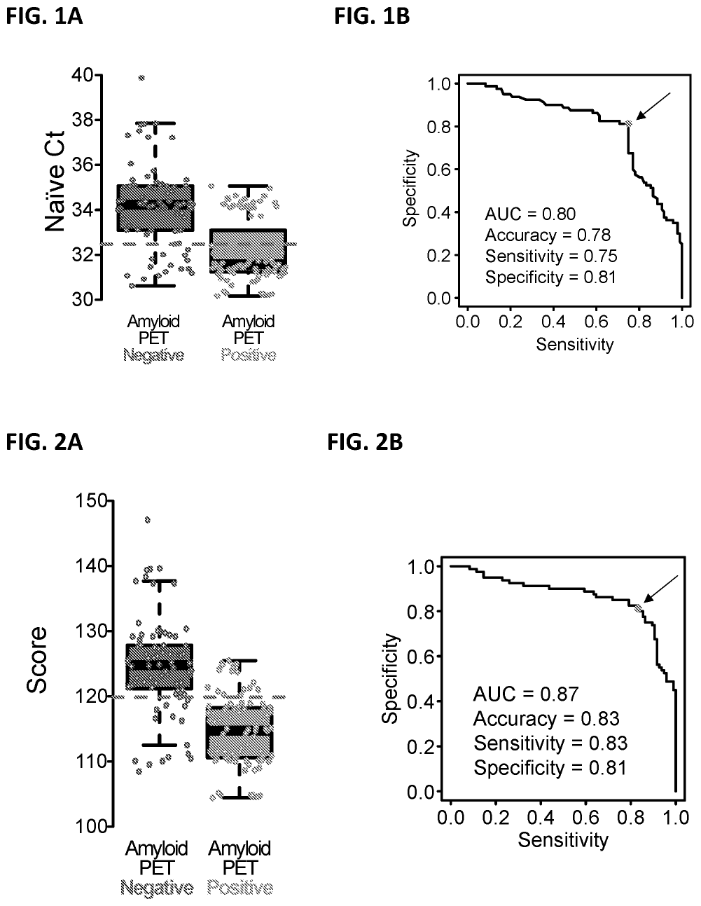

FIG. 1A provides the naïve cycle threshold (Ct) value of the miR-485-3p expression in the clinical swab samples. Naïve Ct value is inversely related to the expression value (i.e., higher expression results in lower naïve Ct). As shown, the miR-485-3p expression was statistically higher in the amyloid-β positive swab samples (i.e., from patients with amyloid-β accumulation) compared to the amyloid-β negative swab samples (i.e., from patients without amyloid-β accumulation). The AUC, accuracy, sensitivity, and specificity of the difference in miR-485-3p expression in the swab samples from the two groups were as follows: (i) 0.80, (ii) 0.78, (iii) 0.75, and (iv) 0.81, respectively (see FIG. 1).

The above result suggests that miR-485-3p expression could be used in distinguishing clinical swab samples from patients with or without amyloid-β accumulation.

### Example 5: Comparison of the Diagnostic Capability of Considering Clinical Information Alone or in Combination with miR-485-3p Expression

Several studies have created models that predict the presence or absence of amyloid beta or AD diagnosis using only patient clinical information. See Kim et al., J Alzheimer's Dis 66:681-691 (2018), which is incorporated herein by reference in its entirety. To compare such a model to the methods described herein (i.e., combining one or more clinical information with miR-485-3p expression level), three different statistical methods were used: (i) clinical information only (CFO), (ii) microRNA's (i.e., miR-485-3p) Ct value, and (iii) microRNA's (i.e., miR-485-3p) quantity. For each of the methods, algorithms of a combination of clinical information in all cases were generated. In the CFO method, algorithms were generated using only the clinical information of the patient without microRNA information. For the Ct value and quantity method, a combination of Ct value and quantity of microRNA was added to the CFO method, respectively. Then, the AUCs of the resulting algorithms were compared and ranked.

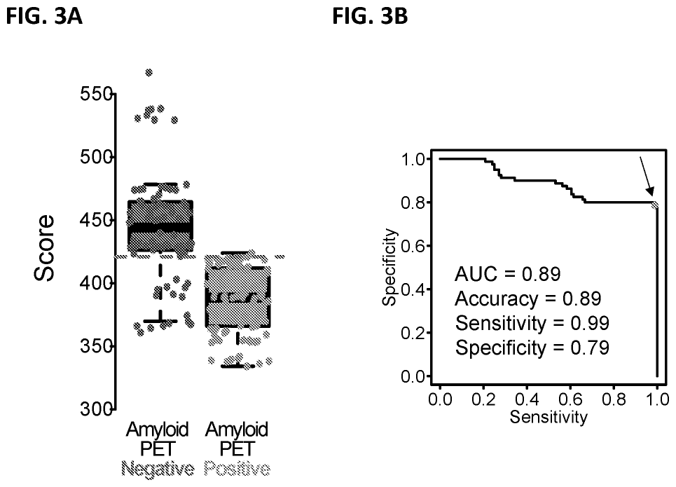

As shown in FIG. 3A, only 58 algorithms out of 1,956 possible algorithms ranked 1^{st} when only combinations of clinical information were considered. Bulk of the algorithms ranked 3^{rd}. In contrast, using the quantity statistical method, all algorithms except 60 (1,896 algorithms) won 1^{st} place. As for the method using Ct value, all algorithms except 1100 (1,856 algorithms) were ranked 2^{nd}. In addition, when the overall AUC values were considered, methods involving the combination of the clinical information with Ct value or quantity value were significantly better compared to methods using clinical information only. The results shown here demonstrate the superiority of the diagnostic methods disclosed in the present disclosure compared to those that exist in the art using clinical information alone.

### Example 6: Analysis of the Effect of Age on Using miR-485-3p Expression to Detect Amyloid-β Accumulation in Human Clinical Swab Samples

To better assess the effect that different clinical information has on the ability of using miR-485-3p expression to diagnose cognitive disorders disclosed herein (e.g., Alzheimer's disease), the relationship between patient's age and miR-485-3p expression in human oral-derived cell free exosomes (HOCFE) was assessed.

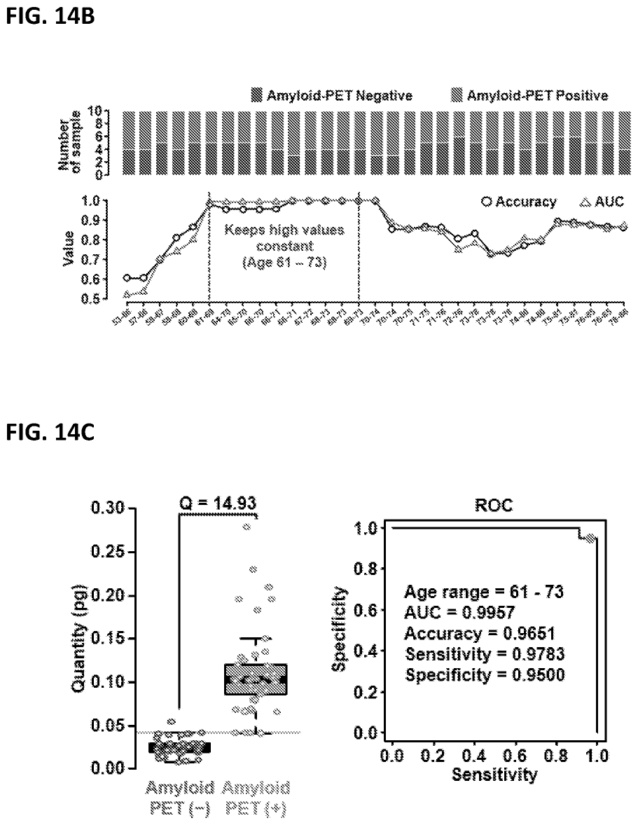

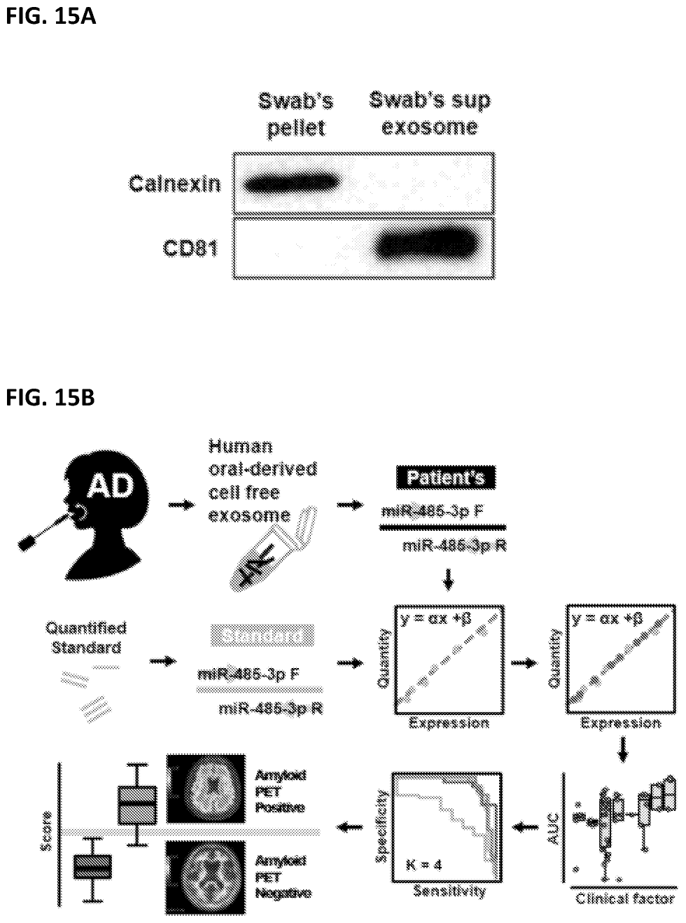

As shown in FIG. 14A, the expression of miR-485-3p in HOCFE decreased as the patient's age increased. This inverse relationship remained statistically significant, even when the HOCFE samples were divided into amyloid PET negative and positive patients. Moreover, the inverse relationship progressed more steeply among amyloid PET positive patients. But among amyloid PET negative patients, miR-485-3p expression showed greater statistical significance with age.

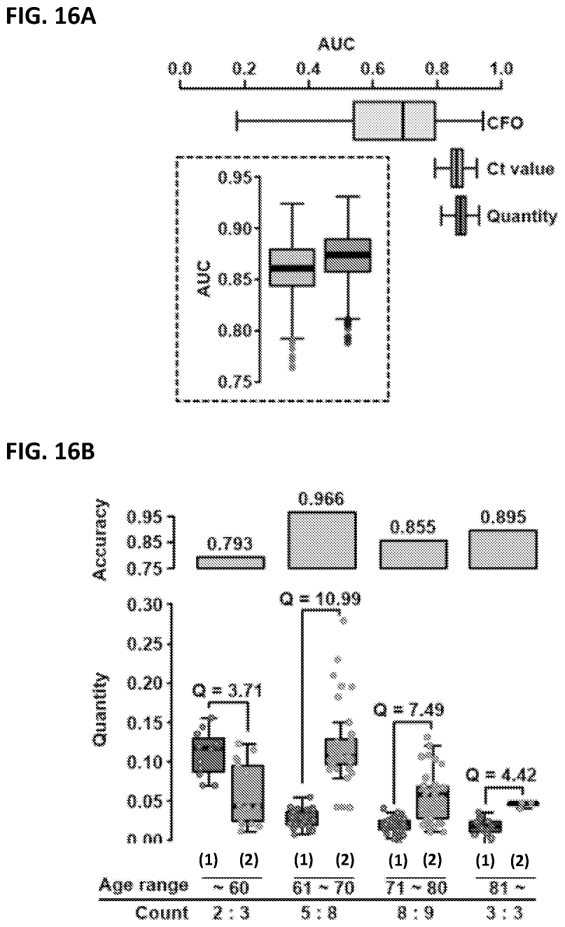

Next, based on the above results, the ability of using miR-485-3p expression to predict amyloid-O accumulation in patients of different age groups was assessed. The patients were divided into the following groups: (i) 60 years old or less (“˜60”); (ii) 61-70 years old (“61˜70”); (iii) 71-80 years old (“71˜80”); and (iv) 81 years old or greater (“81˜”). As shown in FIG. 16B, in all age groups, there was a statistically significant difference in miR-485-3p expression between patients with confirmed amyloid-β accumulation (i.e., amyloid PET positive) and patients confirmed not to have amyloid-β accumulation (i.e., amyloid PET negative). Interestingly, within the less than 61 years old group, there was an increased expression of miR-485-3p among the amyloid PET negative patients compared to amyloid PET positive patients. The relationship was reversed for all other age groups (i.e., greater miR-485-3p expression in the amyloid PET positive subjects). Not to be bound by any one theory, this phenomenon could be due to the sharp increase in the expression of miR-485-3p in patients with amyloid beta accumulation over a certain age. The phenomenon appeared to begin developing in patients over 60 years old and decreased with increasing age. In contrast, in patients without amyloid beta accumulation (i.e., amyloid PET negative), miR-485-3p expression rapidly decreased from the age of 60 or older, and maintained the reduced level with age. The imbalance of miR-485-3p expression with age affected the accuracy of amyloid-β accumulation prediction and had the highest statistical significance in patients in their 60 s (see FIG. 16B). Table 8 (below) provides the specificity and sensitivity values for each of the age groups, which was used to determine

TABLE 8
Formula for Various Algorithms

| Age Range | Specificity | Sensitivity |
|---|---|---|
| 50 s | 0.9167 | 0.6471 |
| 60 s | 0.8462 | 1.0000 |
| 70 s | 0.9744 | 0.6486 |
| 80 s | 0.8750 | 1.0000 |

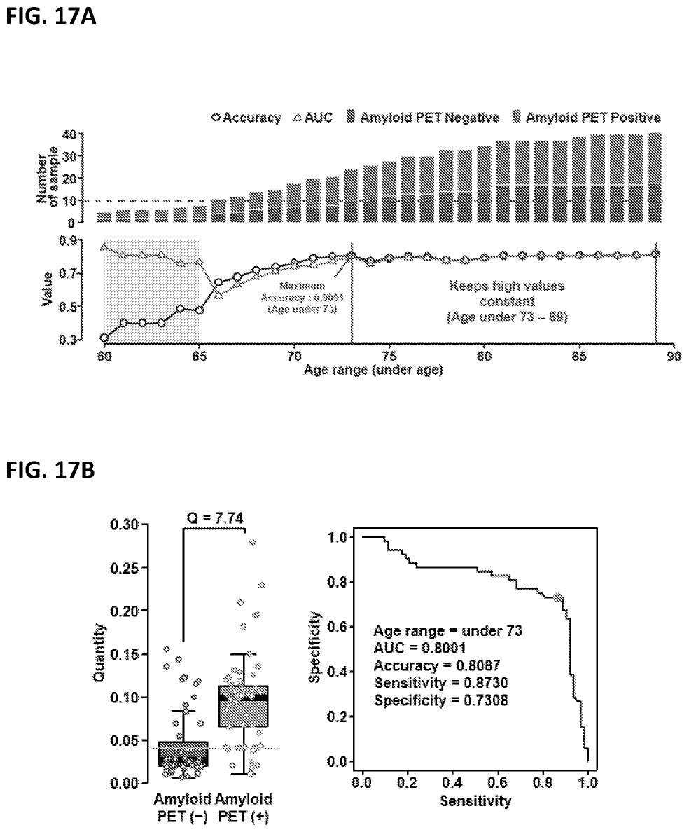

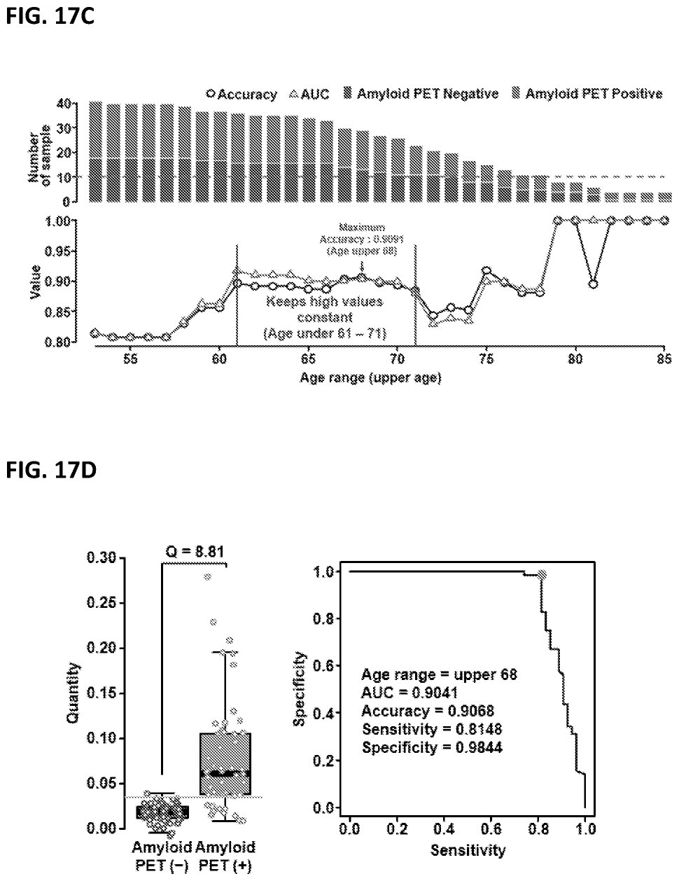

To assess whether the use of miR-485-3p expression to diagnose amyloid-O accumulation was most accurate within certain age groups, the criteria for age were set above or below a certain age group. As shown in FIGS. 17A and 17B, the highest accuracy and AUC were observed in the 73 years or less age group. Within the 65 years or less age group, the expression of miR-485-3p was reversed between groups with or without amyloid beta accumulation, and had low accuracy. The above trend was confirmed in a separate independent experiment with higher age criteria, which showed that the high accuracy and AUC were consistently maintained in patients over 71 years in age, and showed a pattern of rapidly decreasing in the patient group over 72 years old. In groups over 79, the number of samples decreased to 10 or less, and the accuracy fluctuated significantly (see FIG. 17C). Comprehensive test results of two criteria (above or below a certain age) showed that the results of the criteria above a certain age were higher in AUC and accuracy than those under a certain age (see FIGS. 17B and 17D).

As further demonstration of the diagnostic accuracy of miR-485-3p expression within specific age groups, patients were sorted based on age (low to high), and then ten patients were chosen by sliding window to measure AUC and accuracy (see FIG. 14C). Methods of using slide window for such analysis are known in the art (see, e.g., coleoguy.blogspot.com/2014/04/sliding-window-analysis.html, which is incorporated herein by reference in its entirety). Such an approach reduced any variations resulting from sample size for the different age groups. As shown in FIG. 14C, there was greater accuracy and AUC among patients 61 years and younger compared to patients that were 73 years old. Within the 61-73 age group, the accuracy of miR-485-3p expression to predict amyloid-β accumulation was close to 100%.

### Example 7: Analysis of the Effect of Other Clinical Information on Using miR-485-3p Expression to Detect Amyloid-β Accumulation in Human Clinical Swab Samples

To improve the diagnostic ability of using miR-485-3p expression to detect amyloid-β accumulation, the following clinical information were gathered from the patients associated with the clinical swab samples and assigned a value: (i) age, (ii) gender (male=1; female=2), (iii) education year (0-16), (iv) APOE genotype (E2/E3=1; E3/E3=1; E2/E4=2; E3/E4=2; and E4/E4=4), and (v) mini mental state examination (MMSE) score (see Table 3, above). Then, varying combinations of the miR-485-3p expression (i.e., naïve cycle Ct value) and one or more of the above clinical information were created, and the diagnostic accuracy of each of the combinations was determined.

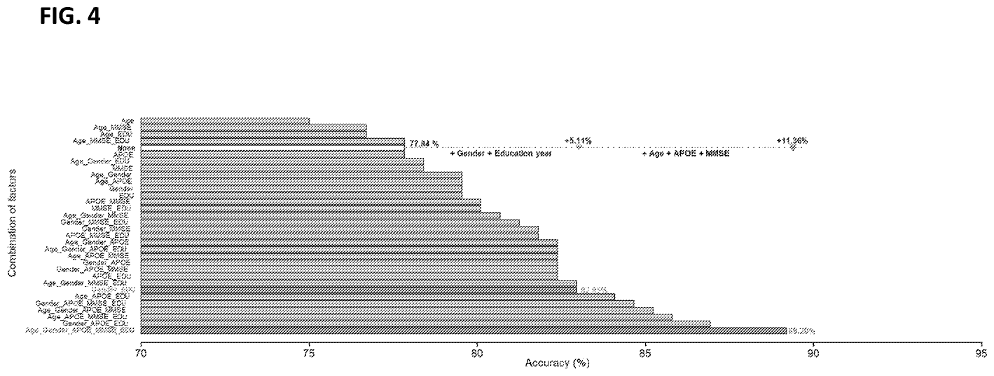

FIG. 4 provides the diagnostic accuracy for the different possible combinations. As shown, the greatest accuracy was observed when miR-485-3p expression in the clinical swab samples was assessed in combination with all the additional clinical information tested (i.e., age, gender, education year, APOE genotype, and MMSE score). When all of the factors were combined, the accuracy was 89.20%, 11.11% higher than when miR-485-3p expression alone was used (compare FIGS. 3A and 3B to FIGS. 1A and 1).

To further assess the ability of using the combination of all the clinical information tested in detecting amyloid-β accumulation, the following formula was used to establish a score for the different clinical swab samples: (Naïve CT×(Age×V1_{Age}+V2_{Age}))×(Gender×V1_{Gender}+V2_{Gender})×(APOE×V1_{APOE}+V2_{APOE})×(MMSE×V1_{MMSE}+V2_{MMSE})×(Education year×V1_{EDU}+V2_{EDU}), wherein V1 and V2 are regression coefficient values associated with the specific additional clinical information. As shown in FIG. 3A, there was a statistical difference in the score of clinical swab samples from patients without amyloid-β accumulation (left) and from patients with amyloid-β accumulation (right).

While the above results suggest the importance of using all of the clinical information tested, additional costs are required to determine a patient's APOE genotype and MMSE score. Therefore, in the interest of providing a less costly means of diagnosing cognitive disorders, the diagnostic accuracy of the different combinations that exclude both APOE genotype and MMSE score were compared. As shown in FIG. 4, the combination of miR-485-3p expression, gender, and education year had the greatest accuracy among combinations that excluded APOE genotype and MMSE score. The accuracy of this combination was 82.95%, 5.11% higher than when no additional clinical information was considered (compare FIGS. 2A and 2B to FIGS. 1A and 1).

To assess the diagnostic ability of this specific combination (i.e., miR-485-3p expression, gender, and education level), the following formula was used to establish a score for the different clinical swab samples: (Naïve Ct×(Gender×V1_{Gender}+V2_{Gender}))×(Education year×V1_{EDU}+V2_{EDU}), wherein V1 and V2 are regression coefficient values associated with the specific additional clinical information. As shown in FIG. 2A, the score for the amyloid PET positive swab samples (i.e., from patients with amyloid-β accumulation) was significantly lower compared to clinical swab samples from patients without amyloid-β accumulation.

The above results collectively demonstrate that the combination of additional clinical information (e.g., gender and education level) can improve the ability of using miR-485-3p expression to distinguish clinical swab samples from patients with or without amyloid-R accumulation.

### Example 8: Addition Analysis of the Diagnostic Capability of Combining miR-485-3p Expression and Clinical Information

Further to the results provided in Example 7 above, additional algorithms or formulae were constructed to confirm the ability of combining miR-485-3p expression with one or more clinical information in diagnosing cognitive disorders (e.g., Alzheimer's disease). In particular, the first algorithm combined age, gender, and education with miR-485-3p expression (referred to herein as “pre-DX”; see Table 9). The second algorithm combined age, gender, education, APOE genotype, and MMSE score with miR-485-3p expression (referred to herein as “pro-DX1”; see Table 9). The third algorithm combined age, gender, education, APOE genotype, MMSE score, and clinical dementia rating (CDR) score with miR-485-3p expression (referred to herein as “pro-DX2”; see Table 9).

TABLE 9
Formula for Various Algorithms

| Algorithm Type | Calculation formula |
|---|---|
| Quantity | Score = (Quantity of miR-485-3p + 0.007) × 10⁴ |
| Pre-DX | Score = ((Quantity of miR-485-3p × (3.5e−02 × Education year⁵ + |
| | 5.2e−02 × Education year⁴ −2e−02 × Education year³ + 2.8e−03 × |
| | Education year² − 1.6e−04 × Education year + 3.1e−06) × |
| | (−0.0027 × Gender + 0.008)) + 7e−0.7) × 10⁸ |
| Pro-DX1 | Score = ((Quantity of miR-485-3p × (3.5e−02 × Education year⁵ + |
| | 5.2e−02 × Education year⁴ −2e−02 × Education year³ + 2.8e−03 × |
| | Education year² − 1.6e−04 × Education year + 3.1e−06) × (0.001 × |
| | Numerical APOE genotype + 0.002) × (−4e−07 × MMSE + 2.1e−05) × |
| | (−3.27 × Numerical Gender + 1.77e−10)) + 8e−17) × 10¹⁸ |
| Pro-DX2 | Score = ((Quantity of miR-485-3p × (0.034 × CDR + 0.06) × (0.0015 × |
| | Numerical APOE genotype + 0.0016) × (−7e−07 × MMSE + 3.48e− |
| | 05) × (1.85e−l l × Education year + 1.51e−10) × (−7.6e−20 × |
| | Numerical Gender + 2.2e−19)) + 7e−31) × 10³³ |
| Quantity | Score = (Quantity of miR-485-3p + 0.007) × 10⁴ |
| (Age > 60) | |
| Pre-DX | Score = ((Quantity of miR-485-3p × (−0.0188 × Numerical Gender + |
| (Age > 60) | 0.08)) + 0.001) × 10⁴ |
| Pro-DXl | Score = ((Quantity of miR-485-3p × (−0.0188 × Numerical Gender + |
| (Age > 60) | 0.08) × (−6.45e−05 × MMSE + 0.004) × (1.345e−06 × Numerical |
| | APOE genotype + 5.86e−06)) + 4e−6) × 10¹³ |
| Pro-DX2 | Score = ((Quantity of miR-485-3p × (0.033 × CDR + 0.055) × |
| (Age > 60) | (−7.65e−05 × MMSE + 0.005) × (−2.87e−06 × Numerical Gender + |
| | 1.456e−05) × (2.77e−5 × APOE + 6e−11)) + 9e−23) × 10²⁴ |

Before constructing the above algorithms, an analysis was performed to identify a suitable dimensional level when applying the regression modeling method to the quantitative value of miR-485-3p for each clinical information. Quantitative value of miR-485-3p and regression modeling were performed up to 11 dimensions for each clinical information, and reproducibility was confirmed by random sampling 100 times for each dimension. In 100 random sampling experiments, the number of experiments (simulations) for which p value was significant was counted, AUC was measured for each experiment, and the error rate was calculated by dividing the deviation value of AUC by the average value of AUC. The dimension with the following properties was selected: (i) statistically significant experimental results (i.e., number of simulations out of total 100 simulations with a p-value<0.05), (ii) relatively high AUC, and (iii) relatively low error rate (see FIGS. 18A-18F and 19A-19F).

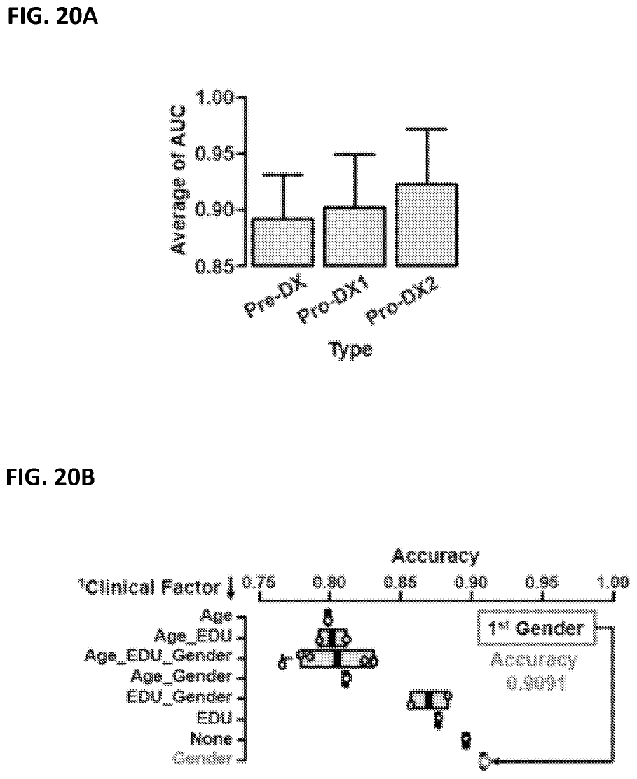

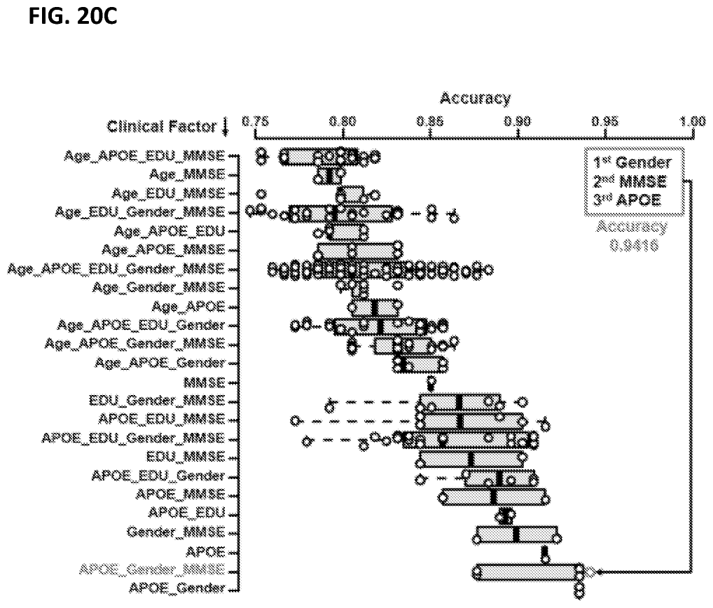

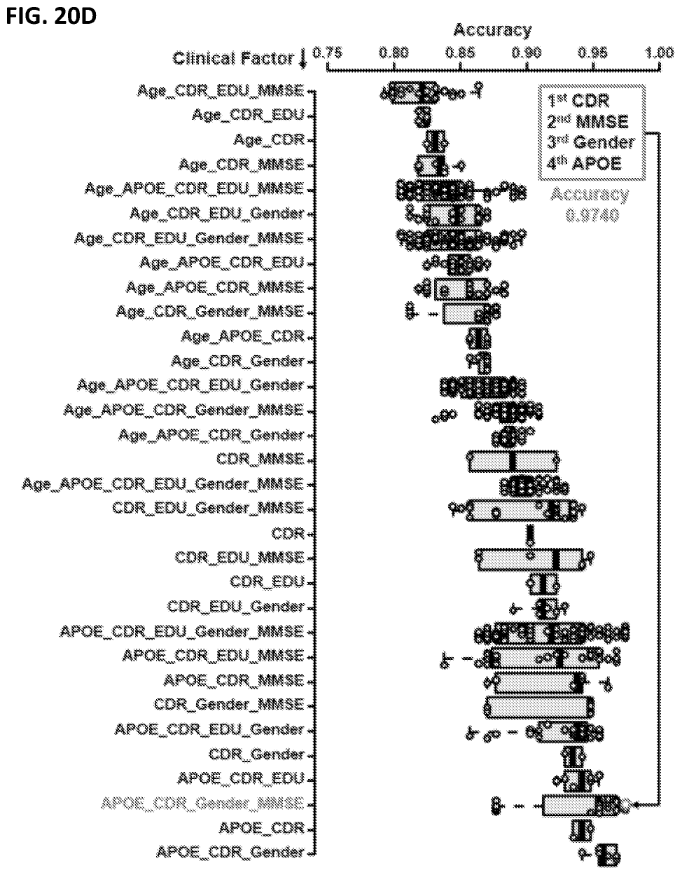

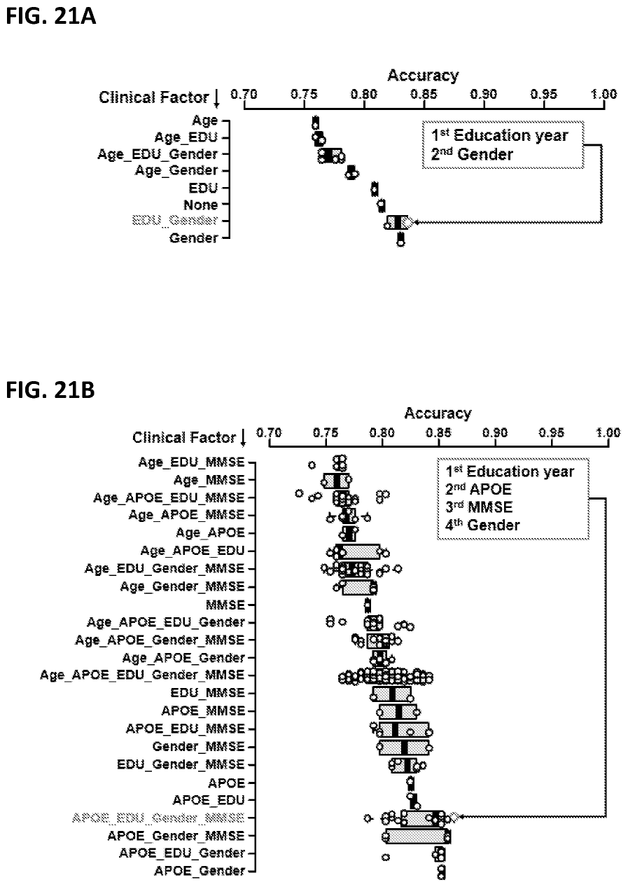

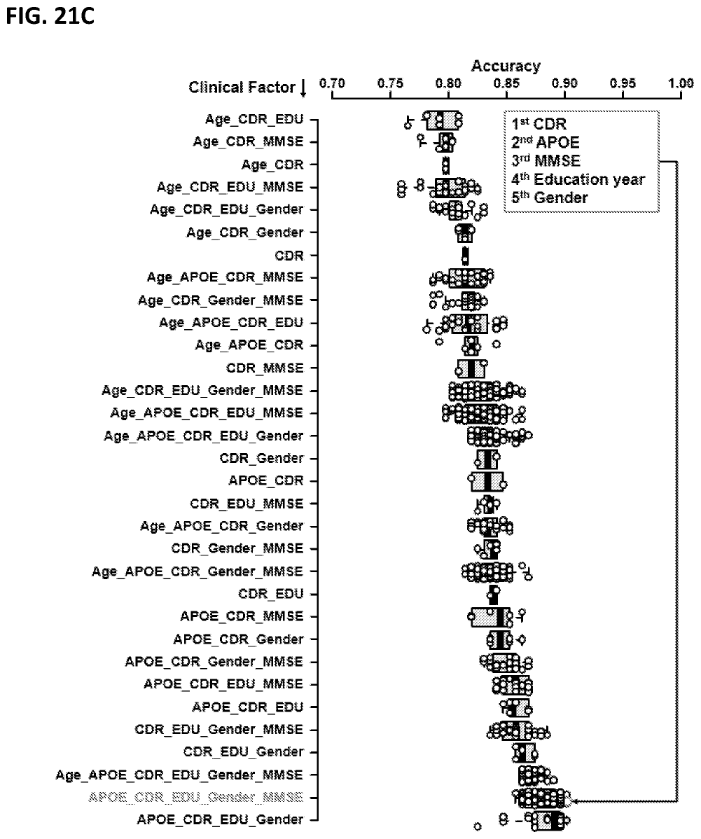

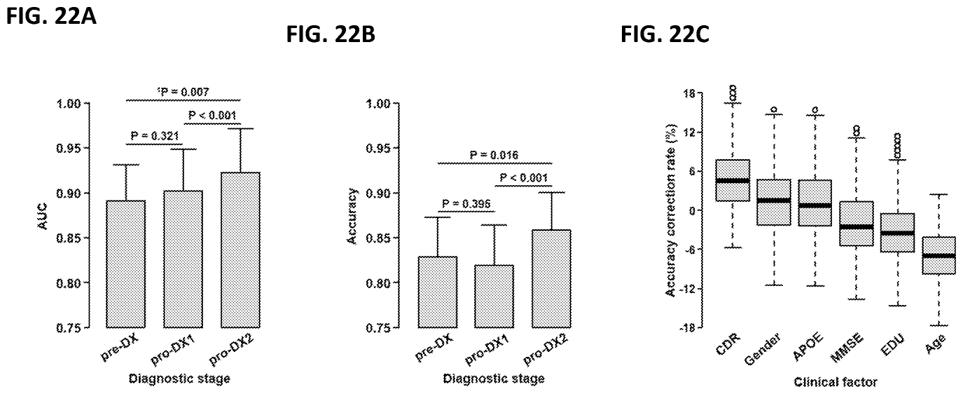

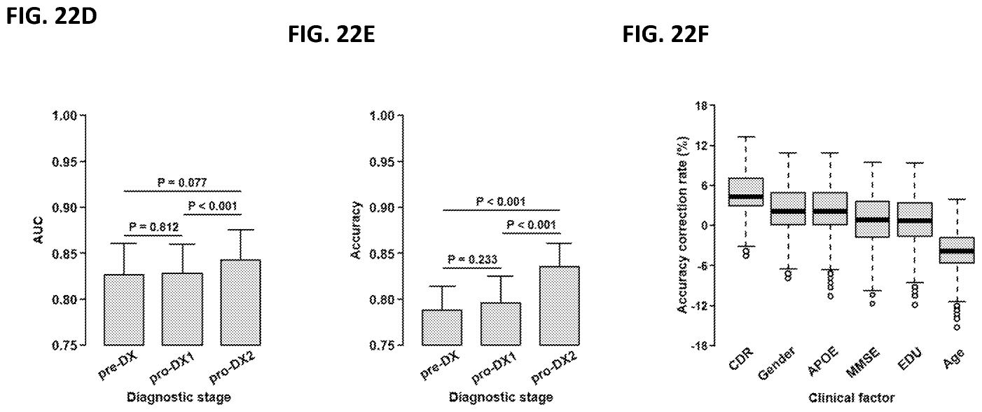

Once the dimensional values were selected, the different clinical information were applied to the above algorithms, and then, accuracy determined for the different combinations. As shown in FIGS. 20B, 20C, and 20D, while the particular clinical information were the same for a given algorithm, the order in which the clinical information was applied to have an effect. For the pre-DX algorithm, the highest accuracy was observed when gender alone was combined with miR-485-3p expression (see FIG. 20B). For the pro-DX1 algorithm, the highest accuracy was observed when gender, MMSE score, and APOE genotype (in the recited order) was applied to the algorithm in combination with miR-485-3p expression (see FIG. 20C). For the pro-DX2 algorithm, the highest accuracy was observed when CDR score, MMSE score, gender, and APOE genotype (in the recited order) was applied to the algorithm in combination with miR-485-3p expression (see FIG. 20D). Considering the overall AUC value for each of the above algorithms, the pro-DX2 algorithm, which applied the most clinical information types, recorded the highest average AUC value (see FIG. 20A). Similarly, the algorithm with the highest accuracy (0.9740) was also measured using the pro-DX2 algorithm (see FIG. 20D). Similar results were observed using samples from patients under 61 years old only (see FIGS. 21A-21C) or over 60 years old only (see FIGS. 22A-22F).

In addition to the above, the effect of individual clinical information on accuracy was assessed for the above algorithms. To do so, the accuracy correction rate was determined as described in Example 1. As shown in FIGS. 22C and 22F, the CDR score was associated with the great accuracy. Interestingly, age did not improve accuracy, and in fact, appeared to lower the accuracy. Not to be bound by any one theory, and as described earlier, such result could be due to the rapid rise and fall of miR-485-3p expression within the amyloid-PET positive patient group.

### Example 9: Combining Clinical Information with miR-485-3p Expression for Diagnosing Cognitive Disorders

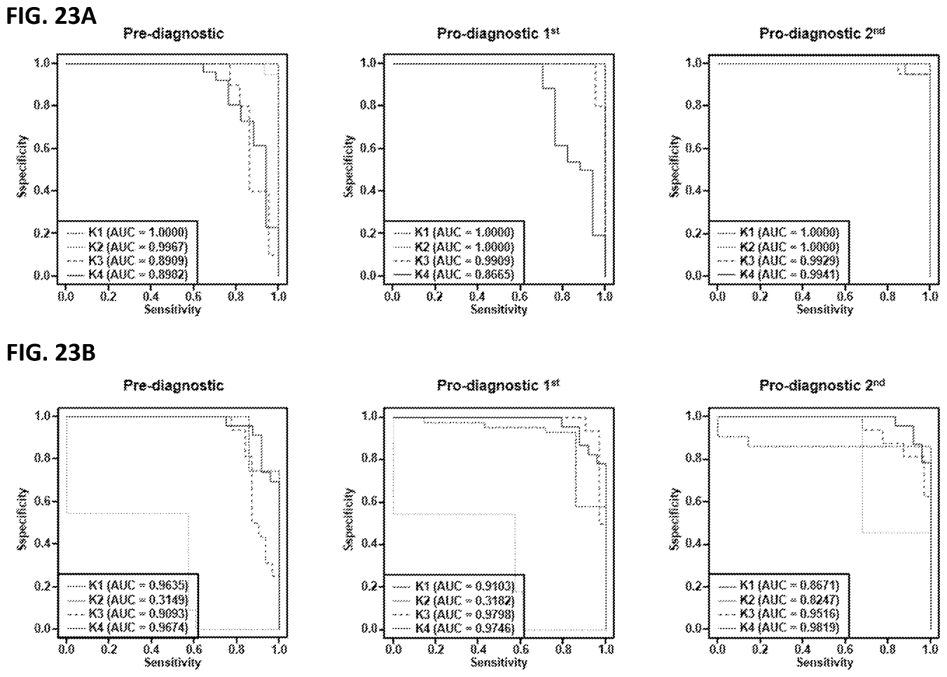

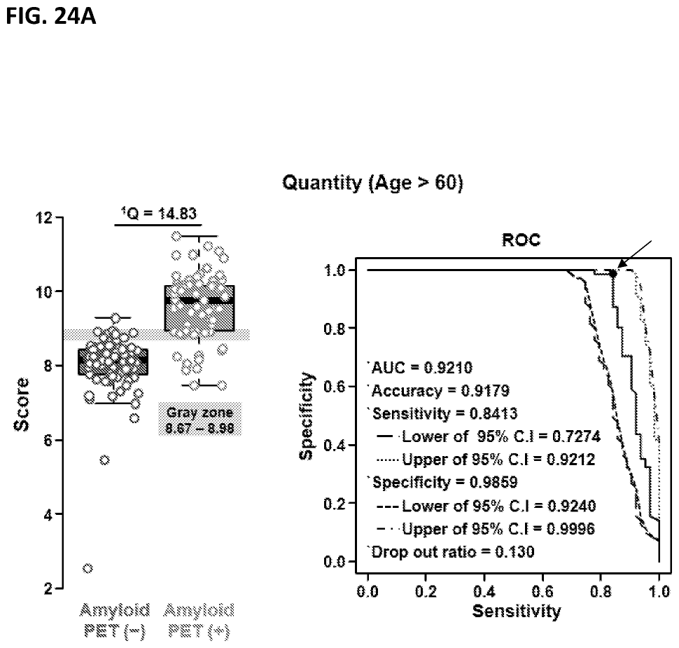

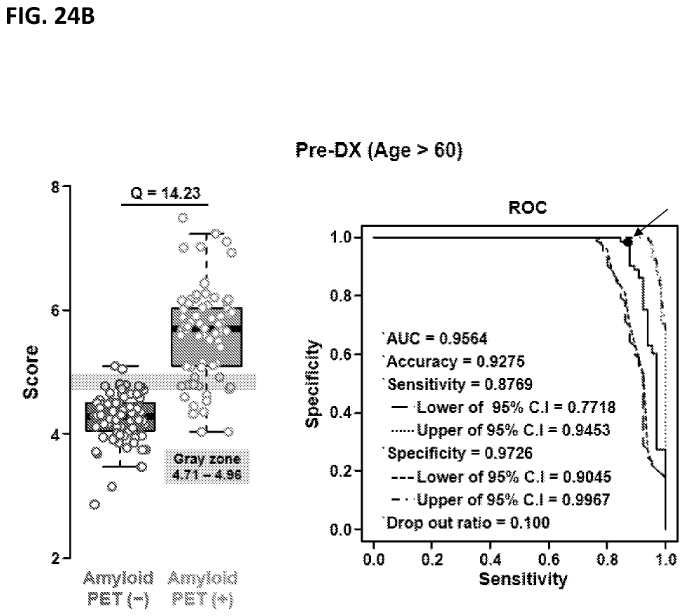

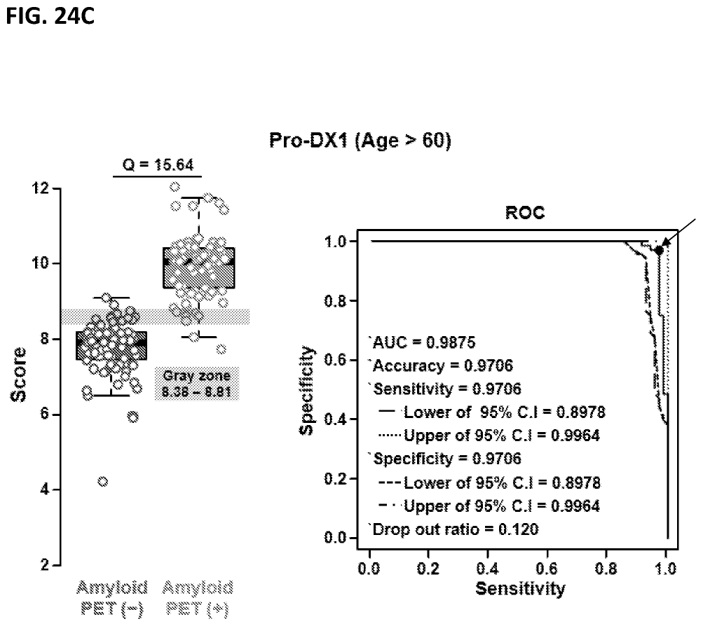

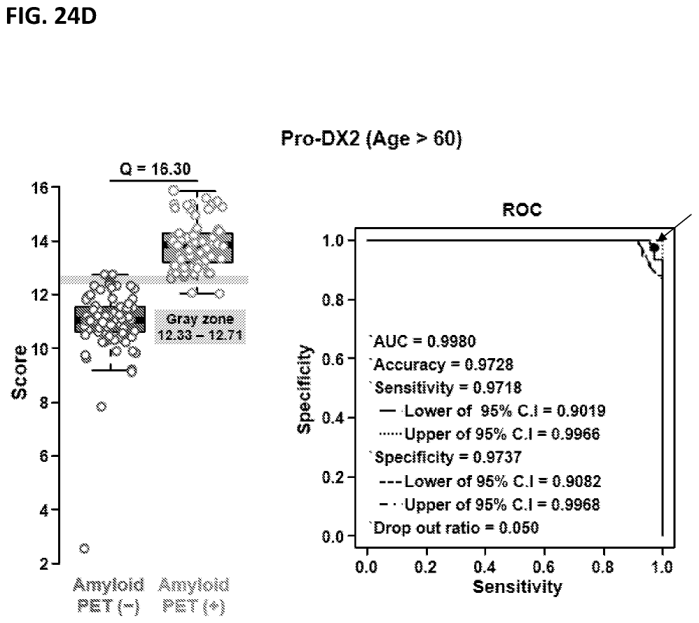

To verify and identify the algorithms with the highest accuracy, the K-fold cross validation method was used. See, e.g., Jung et al., J Nonparametr Stat 27(2):167-179 (2015), which is incorporated herein by reference in its entirety. Briefly, the patient samples were equally divided into four groups. Three of the groups were used as trial set, while one of the group was used as a validation set. Then, both identification and verification was performed for each of the groups for a total of four times. After the verification, high AUC values (up to 0.86) were achieved for all of the three algorithms described earlier (i.e., pre-DX, pro-DX1, and pro-DX2). The K-th model with the highest AUC value was selected as the final model for each algorithm (see FIGS. 23A and 23B). The calculation formula of each algorithm is shown in Table 9 (above). The results for the different algorithms are provided in FIGS. 24A-24D.

The above results suggest that the combination of different clinical information and miR-485-3p expression could be useful as a diagnostic tool for certain cognitive disorders (e.g., Alzheimer's disease).

### Example 10: Real-Time PCR Analysis of miR-485-3p Expression in Human Plasma Samples

Next, the ability of using miR-485-3p expression to detect amyloid-O accumulation in human plasma samples was assessed. miR-485-3p expression was measured using real-time PCR as described earlier, e.g., in Example 2.

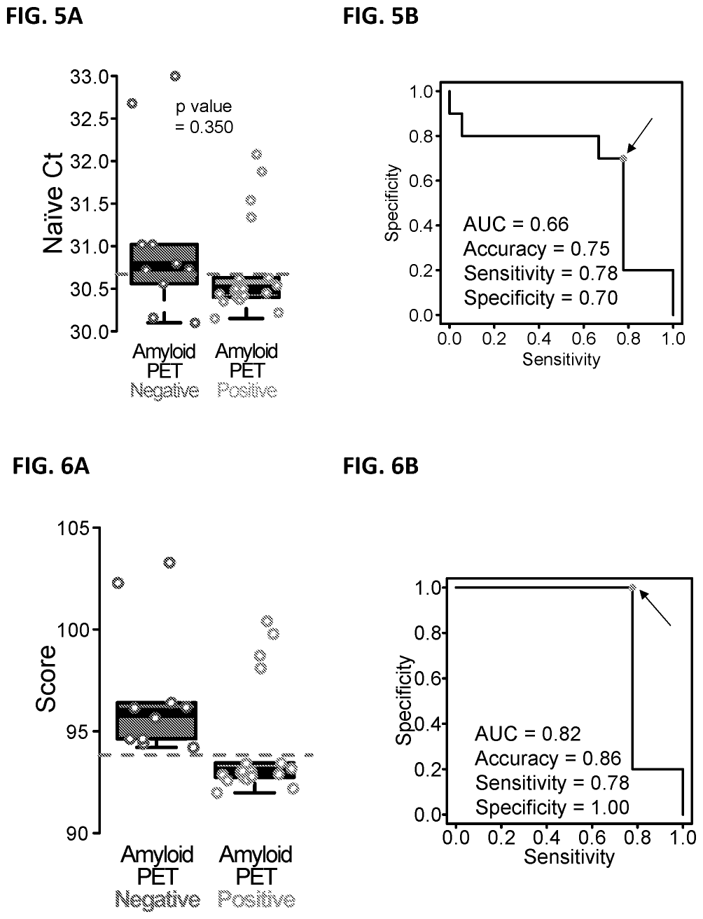

As shown in FIG. 5A, unlike the clinical swab samples, there was no significant difference in the naïve cycle threshold (Ct) values between the amyloid PET negative (i.e., from patients without amyloid-β accumulation) and amyloid PET positive (i.e., from patients with amyloid-β accumulation) plasma samples. Compared to the clinical swab samples, the AUC, accuracy, sensitivity, and specificity values were all significantly lower (compare FIG. 5B and FIG. 1).

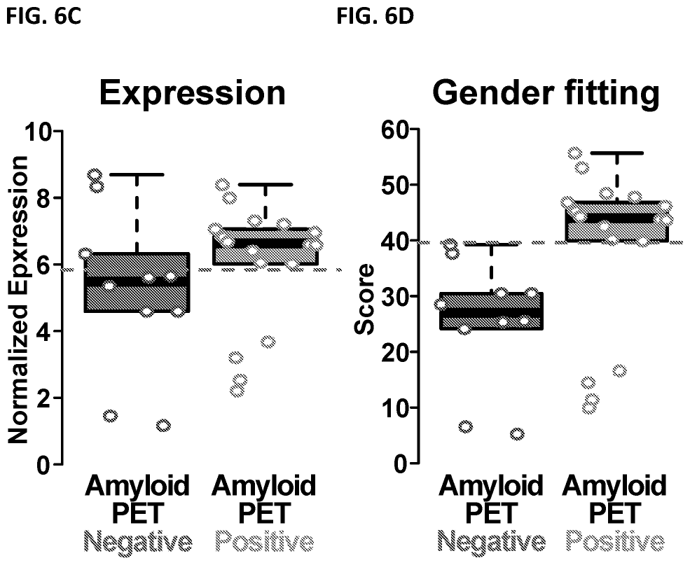

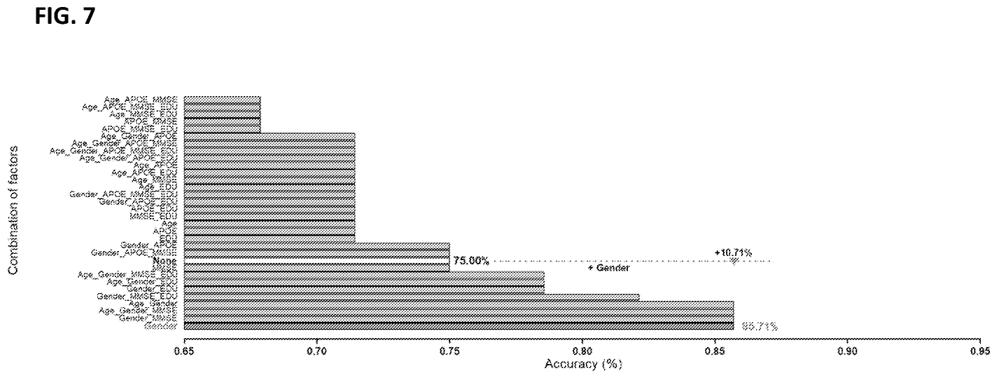

However, when miR-485-3p expression in human plasma samples was combined with patient's gender information alone, there was noticeable difference between the plasma samples from patients with and without amyloid-β accumulation. For instance, using the following formula, a score was established for the different plasma samples: (Naïve CT×(Gender×V1_{Gender}+V2_{Gender})), wherein V1 and V2 are regression coefficient values associated with the additional clinical information. As shown in FIG. 6A, a statistical difference was observed between the amyloid PET negative and amyloid PET positive plasma samples. Similarly, the values for AUC, accuracy, sensitivity, and specificity were all significantly higher compared to corresponding values when miR-485-3p expression alone was used from the plasma samples (compare FIG. 6B and FIG. 5B). The miR-485-3p expression was normalized from the assay and compared in FIG. 6C. The gender fitting scores are also calculated and plotted as shown in FIG. 6D. Unlike FIGS. 6A and 6B, which indicate lower number on the Y axis meaning higher miR-485-3p expression, FIGS. 6C and 6D are plotted to show that a higher miR-485-3p expression has a higher number on the Y axis. FIG. 7 provides the diagnostic accuracy for the varying combinations of miR-485-3p expression in the plasma samples and one or more of the additional clinical information described earlier in Example 2 (i.e., age, gender, education year, APOE genotype, and MMSE score) (see also Tables 3A and 3B, above). As shown, when using human plasma samples, the combination of miR-485-3p expression and gender alone resulted in the highest accuracy (i.e., 85.71%).

The above results suggest that miR-485-3p expression in human plasma samples could also be used as a diagnostic marker for amyloid-β accumulation when combined with other clinical information (e.g., patient's gender).

### Example 11: Analysis of the Relationship Between Amyloid-β Accumulation and miR-485-3p Expression

To further assess the relationship between amyloid-β accumulation and miR-485-3p expression, human-derived oral epithelial cells were treated with varying concentrations (i.e., 0, 0.1, 0.5, or 1 μM) either amyloid-β monomer or oligomer. Then, the expression of miR-485-3p was assessed both in the treated cells and in the supernatant of the treated cells using real-time PCR. miR-485-3p expression in the treated cells was measured using two different primers: (i) 5′-GTCATACACGGCTCTCCTCTCT-3′ (referred to herein as “miR-485-3p_FW1”; SEQ ID NO: 94); and (ii) 5′-CATACACGGCTCTCCTCTCTAAA-3′ (referred to herein as “miR-485-3p_FW9”; SEQ ID NO: 52). To measure miR-485-3p expression in the supernatant, the miR-485-3p_FW9 primer was used.

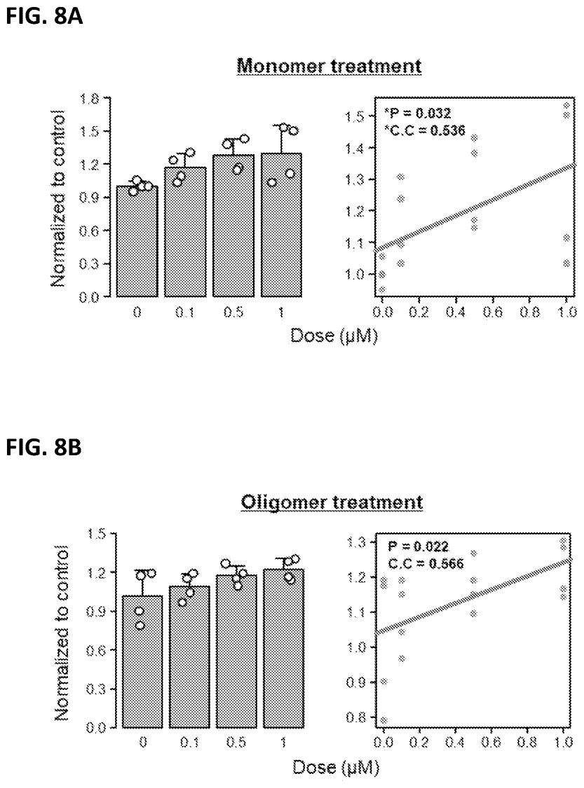

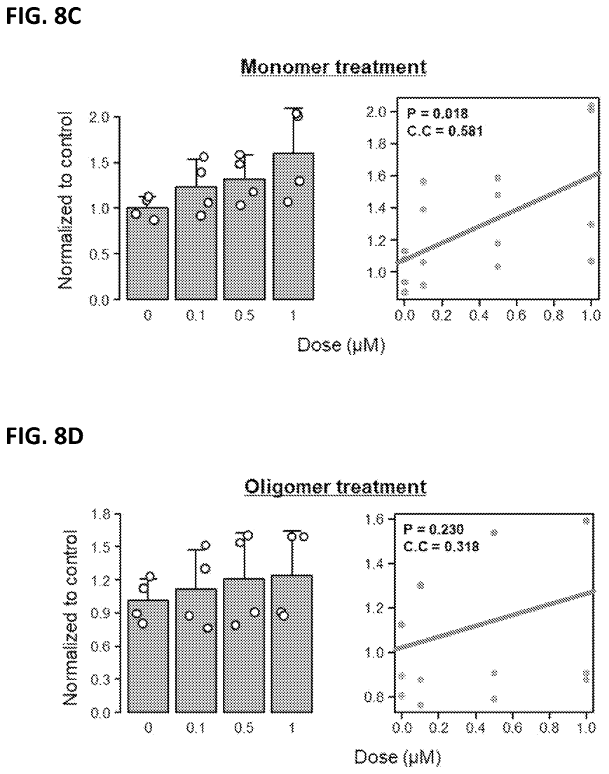

As shown in FIGS. 8A and 8B, with the miR-485-3p_FW1 primer, there was a statistically significant positive correlation between amyloid-β concentration and miR-485-3p expression in cells treated with either the amyloid-β monomer or oligomer. However, with the miR-485-3p_FW9, a statistically significant positive correlation was only observed in the amyloid-β monomer treated cells (see FIG. 8C). In the cells treated with the amyloid-β oligomer, there was a tendency for the miR-485-3p expression to increase with an increase in amyloid-β concentration, but the correlation was not statistically significant (see FIG. 8D).

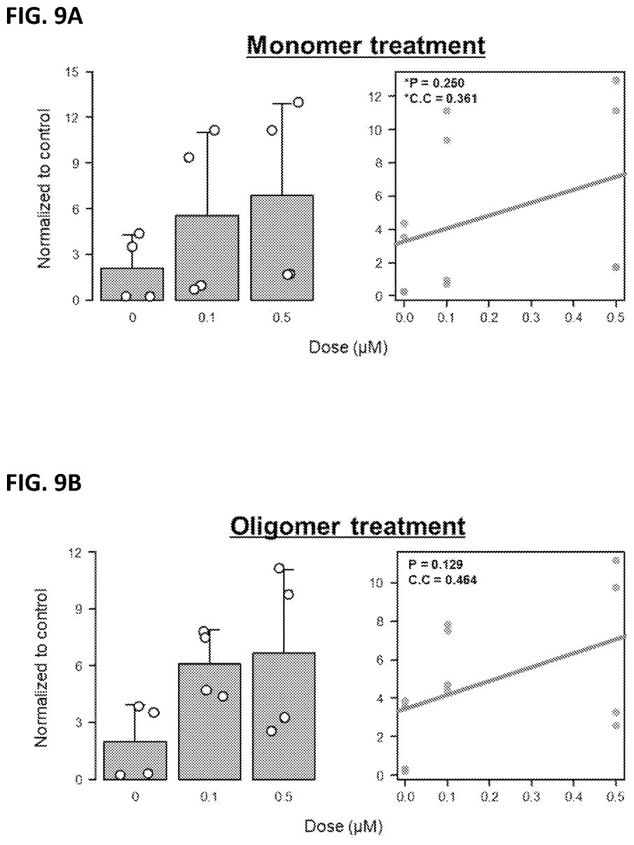

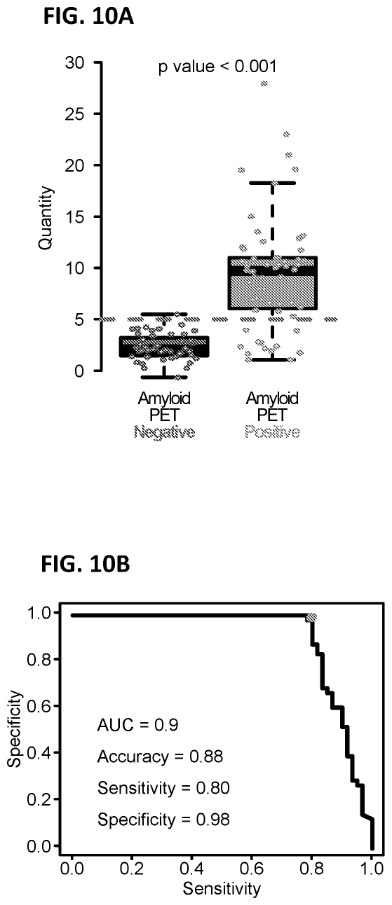

In contrast to the treated cells, there was no significant correlation (only a positive tendency) between amyloid-β concentration and miR-485-3p expression in the supernatant. This was true for both the amyloid-β monomer and oligomer (see FIGS. 9A and 9B).

The above results further demonstrate the relationship between amyloid-β accumulation and miR-485-3p expression, at least in cells, such as oral epithelial cells (e.g., swab samples), confirming that miR-485-3p can be used as a diagnostic marker to identify patients with certain cognitive disorders, such as those associated with amyloid-β accumulation (e.g., Alzheimer's disease).

### Example 12: Diagnosis of Amyloid-β Accumulation Using miR-485-3p Expression

Further to the examples provided above (see, e.g., Example 10), the ability of miR-485-3p expression to diagnose amyloid-β accumulation was further assessed. In particular, using the algorithms described herein (i.e., quantitation, pre-DX, pro-DX1, and pro-DX2; see Table 9, above) and the miR-485-3p expression values in the human oral-derived cell free exosome isolated from oral clinical swab samples, diagnostic scores were generated. Then, using the methods described herein (see, e.g., Example 1), an optimal cut-off value having the highest accuracy was determined, and a gray zone established.

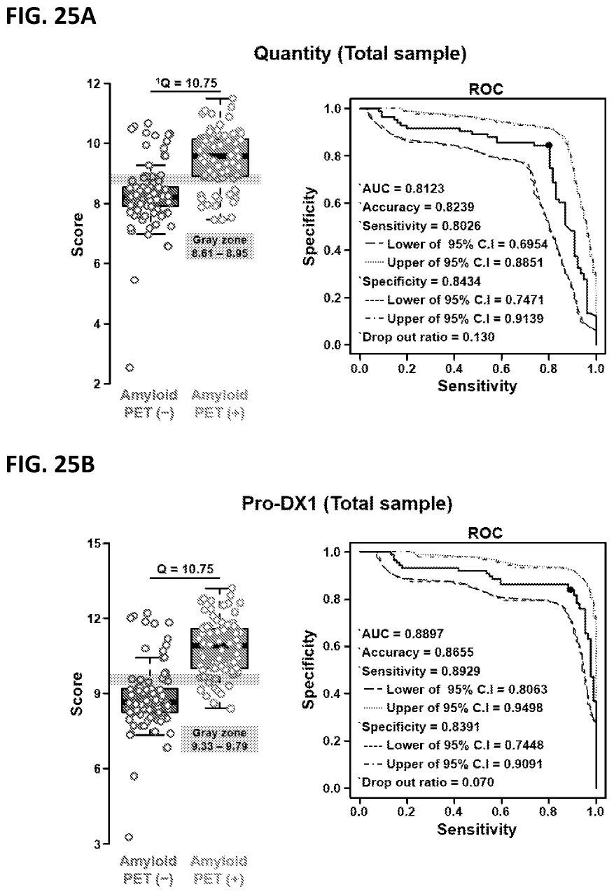

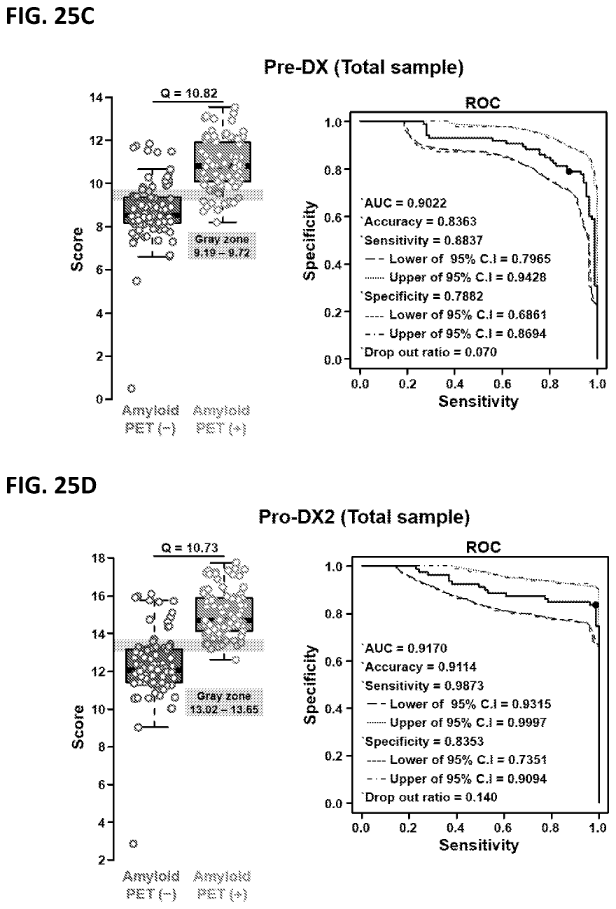

As shown in FIGS. 24A-24D, both high AUC values (0.92 or higher) and high accuracy (0.91 or higher) was achieved using the different algorithms provided. In agreement with earlier data, as the number of different clinical information increased, higher AUC values were observed. Similar results were observed in a separate independent experiment when age was not restricted (see FIGS. 25A-25D). However, when age was considered, relatively high AUC and accuracy were observed among patients over 61 years old (see FIGS. 24A-24D). There was an increase in accuracy of approximately 9% in the quantity diagnostic method (see Table 3A), approximately 9% in the pre-DX method, approximately 11% in the pro-DX1 method, and approximately 6% in the pro-DX2 method. AUC values were also higher in the patient group over the age of 60. In addition, when age was limited to 61 years or older, the sensitivity increased by an average of 2%, and the specificity increased by an average of 15% (see Table 3A).

The above results confirm the diagnostic capability of combining different clinical information and miR-485-3p expression described herein.

### Example 13: Diagnosis of Cognitive Impairment Using miR-485-3p Expression

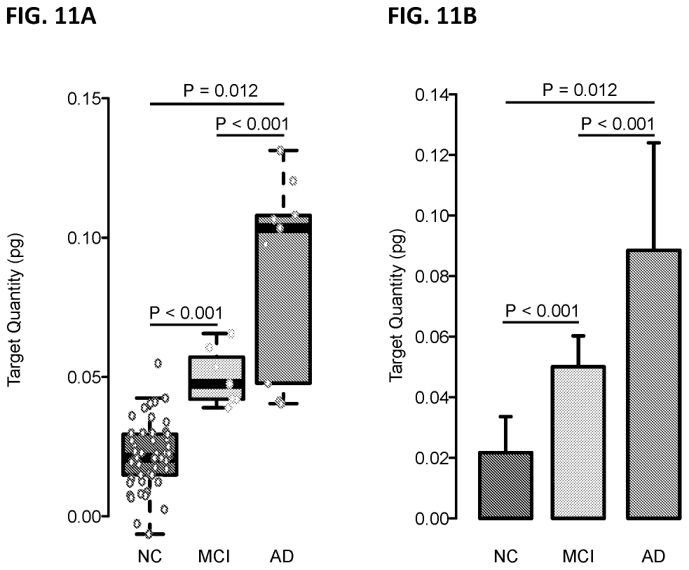

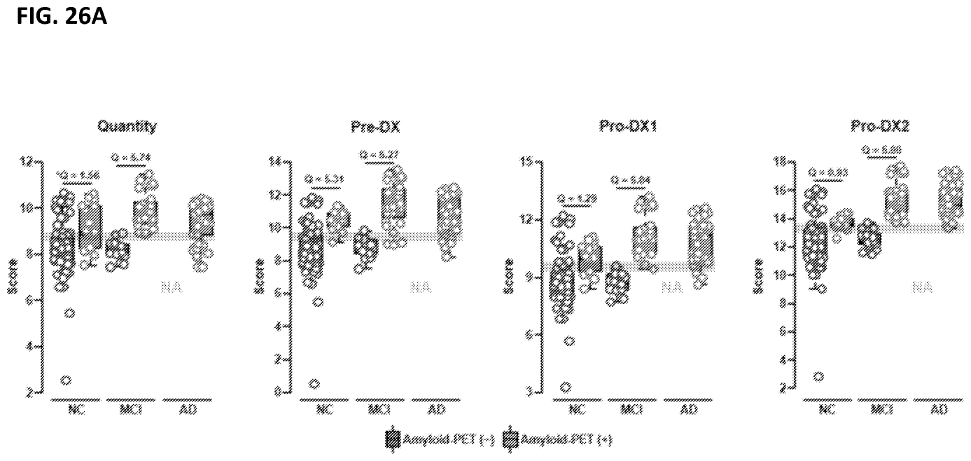

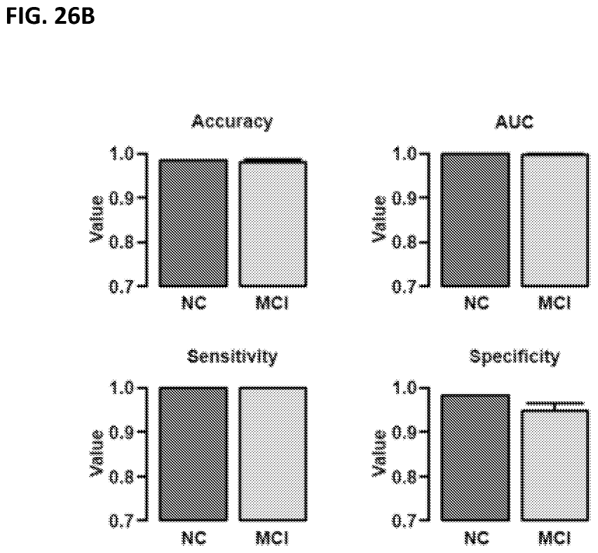

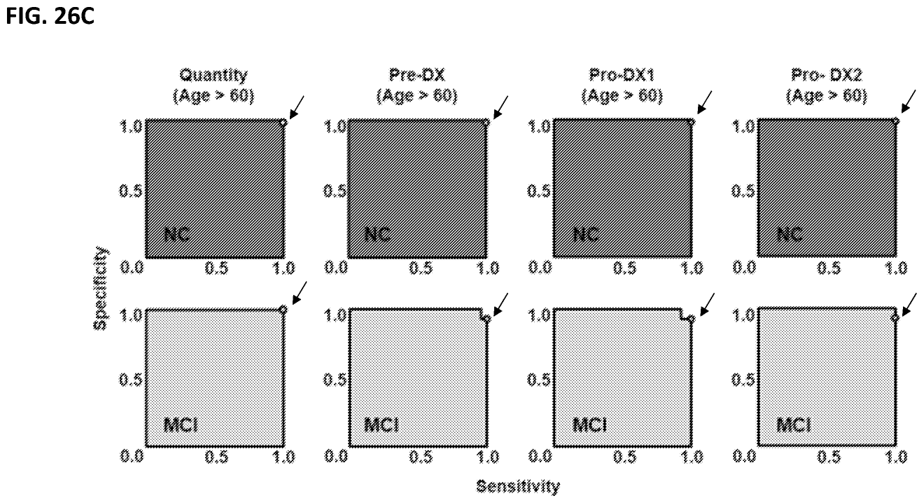

To assess the ability of miR-485-3p expression to predict cognitive impairment, patients were divided based on their Alzheimer's disease diagnosis according to the degree of cognitive impairment (i.e., normal cognitive (NC); mild cognitive impairment (MCI), and Alzheimer's disease (AD)). Although there was some bias between positive and negative amyloid PET patients from the different groups, all of the diagnostic methods shown (i.e., using one of the following algorithms: quantity, pre-DX, pro-DX1, pro-DX2; see Table 9) achieved strong statistical significance (see FIGS. 11A, 111B, and 26A-26C). In particular, the NC and MCI patient groups showed very high statistical accuracy (see FIGS. 26A-26C). The NC patient group showed higher results across all statistical values than the other patient groups. In addition, the group of NC patients, including those under the age of 61, showed relatively low predictive power. The NC patient group included most patients under the age of 61.

The above results demonstrate that the high predictive power of amyloid-β accumulation in the NC and MCI patient groups can be used as a very useful diagnostic criterion to identify patients within the groups that are transitioning to AD onset.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that can be cited throughout this application are hereby expressly incorporated by reference in their entirety for any purpose, as are the references cited therein.

What is claimed is:

1. A method of identifying a human subject afflicted with a cognitive disorder comprising measuring a level of miR-485-3p in a biological sample derived from an epithelial cell or serum of the subject.

2. The method of claim 1, wherein the biological sample is an extracellular vesicle.

3. A method of identifying a subject afflicted with a cognitive disorder comprising measuring a level of miR-485-3p in a biological sample obtained from the subject, wherein the biological sample comprises an extracellular vesicle.

4. The method of claim 3, wherein the extracellular vesicle is obtained from an epithelial cell of the subject.

5. The method of claim 4, wherein the epithelial cell is an oral mucosal epithelial cell.

6. The method of claim 3, wherein the extracellular vesicle is obtained from serum of the subject.

7. The method of any one of claims 2 to 6, wherein the extracellular vesicle comprises a microvesicle.

8. The method of any one of claims 2 to 6, wherein the extracellular vesicle comprises an exosome.

9. The method of any one of claims 1 to 8, wherein the level of miR-485-3p is increased in the subject compared to a reference level (e.g., a miR-485-3p expression level in a subject without a cognitive disorder or a miR-485-3p level prior to having a cognitive disorder in the subject).

10. The method of claim 9, wherein the level of miR-485-3p is increased in the subject by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more, compared to the reference level.

11. The method of any one of claims 1 to 10, further comprising administering a therapy to treat the cognitive disorder.

12. A method of treating a cognitive disorder in a human subject in need thereof comprising administering a therapy to treat the cognitive disorder to a human subject identified as having an increased level of miR-485-3p in a biological sample derived from an epithelial cell or serum of the subject, compared to a reference level (e.g., a miR-485-3p expression level in a subject without a cognitive disorder or a miR-485-3p level prior to having a cognitive disorder in the subject).

13. The method of claim 12, wherein the biological sample is an extracellular vesicle.

14. The method of claim 13, wherein the extracellular vesicle is obtained from an epithelial cell of the subject.

15. The method of claim 14, wherein the epithelial cell is an oral mucosal epithelial cell.

16. The method of claim 13, wherein the extracellular vesicle is obtained from serum of the subject.

17. The method of any one of claims 13 to 16, wherein the extracellular vesicle comprises a microvesicle.

18. The method of any one of claims 13 to 16, wherein the extracellular vesicle comprises an exosome.

19. The method of any one of claims 1 to 18, wherein the level of miR-485-3p in the biological sample is measured using a polymerase chain reaction (PCR) assay.

20. The method of claim 19, wherein the PCR assay comprises a real time PCR.

21. The method of claim 19 or 20, wherein the measuring comprises determining a cycle threshold (Ct) value of miR-485-3p.

22. The method of any one of claims 1 to 21, further comprises measuring an additional factor regarding the subject, wherein the additional factor is selected from age, gender, education year (EDU), apolipoprotein E (APOE) genotype, Mini Mental State Examination (MMSE) score, or any combination thereof.

23. The method of claim 22, wherein the additional factors are gender and education year.

24. The method of claim 22, wherein the additional factor is gender.

25. The method of any one of claims 22 to 24, wherein the gender comprises male or female, and wherein male is associated with a value of 1 and female is associated with a value of 2.

26. The method of any one of claims 22 to 25, wherein the APOE genotype comprises (i) E2/E3, which is associated with a value of 1, (ii) E3/E3, which is associated with a value of 1, (iii) E2/E4, which is associated with a value of 2, (iv) E3/E4, which is associated with a value of 2, or (v) E4/E4.

27. The method of any one of claims 22 to 26, wherein the education year comprises a value between 0 and 16.

28. The method of any one of claims 22 to 27, further comprising calculating a diagnostic score of the subject using the following formula:
(Naïve Ct×(Gender×V1_{Gender}+V2_{Gender}))×(Education year×V1_{EDU}+V2_{EDU}),
wherein V1 and V2 are regression coefficient values associated with the specific additional factor.

29. The method of any one of claims 22 to 27, further comprising calculating a diagnostic score of the subject using the following formula:
(Naïve CT×(Age×V1_{Age}+V2_{Age}))×(Gender×V1_{Gender}+V2_{Gender})×(APOE×V1_{APOE}+V2_{APOE})×(MMSE×V1_{MMSE}+V2_{MMSE})×(Education year×V1_{EDU}+V2_{EDU}),
wherein V1 and V2 are regression coefficient values associated with the specific additional factor.

30. The method of any one of claims 22 to 27, further comprising calculating a diagnostic score of the subject using the following formula:
(Naïve CT×(Gender×V1_{Gender}+V2_{Gender})),
wherein V1 and V2 are regression coefficient values associated with the specific additional factor.

31. The method of any one of claims 1 to 30, wherein the measuring comprises using one or more miR-485-3p primers to amplify the miR-485-3p present in the biological sample.

32. A method of determining a level of miR-485-3p in a subject afflicted with a cognitive disorder, comprising detecting whether the level of miR-485-3p in a biological sample obtained from the subject is increased compared to a reference level (e.g., a miR-485-3p expression level in a subject without a cognitive disorder or a miR-485-3p level prior to having a cognitive disorder in the subject) by amplifying the miR-485-3p present in the biological sample with one or more miR-485-3p primers.

33. The method of claim 32, wherein the level of miR-485-3p is increased in the subject by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more, compared to the reference level.

34. The method of claim 32 or 33, wherein the biological sample comprises a tissue, cell, blood, serum, saliva, or combinations thereof.

35. The method of any one of claims 32 to 34, wherein the biological sample comprises an extracellular vesicle.

36. The method of claim 35, wherein the extracellular vesicle is obtained from an epithelial cell of the subject.

37. The method of claim 36, wherein the epithelial cell is an oral mucosal epithelial cell.

38. The method of claim 35, wherein the extracellular vesicle is obtained from serum of the subject.

39. The method of any one of claims 35 to 38, wherein the extracellular vesicle comprises a microvesicle.

40. The method of any one of claims 35 to 38, wherein the extracellular vesicle comprises an exosome.

41. The method of any one of claims 31 to 40, wherein the miR-485-3p primers comprise miR-485-3p_FW1 (GTCATACACGGCTCTCCTCTCT) (SEQ ID NO: 94), miR-485-3p_FW2 (TCATACACGGCTCTCCTCTC) (SEQ ID NO: 95), miR-485-3p_FW3 (CATACACGGCTCTCCTCTC) (SEQ ID NO: 96), miR-485-3p_FW4 (CATACACGGCTCTCCTCTCTA) (SEQ ID NO: 97), miR-485-3p_FW5 (CATACACGGCTCTCGTCTC) (SEQ ID NO: 98), miR-485-3p_FW6 (CATACACGGCTCTCGTCTCTAA) (SEQ ID NO: 99), miR-485-3p_FW7 (GTCATACACGGCTCTCCTCTCTAA) (SEQ ID NO: 100), miR-485-3p_FW8 (GTCATACACGGCTCTCCTC) (SEQ ID NO: 101), miR-485-3p_FW9 (CATACACGGCTCTCCTCTCTAAA) (SEQ ID NO: 52), miR-485-3p_FW10 (GTCATACACGGCTCTCCTCTG) (SEQ ID NO: 102), miR-485-3p_FW11 (TCATACACGGCTCTCCTCTCT) (SEQ ID NO: 103), miR-485-3p_FW12 (TCATACACGGCTCTCCTC) (SEQ ID NO: 104), miR-485-3p_FW13 (TCATACACGGCTCTCCTCTCTAA) (SEQ ID NO: 105), miR-485-3p_FW14 (CATACACGGCTCTCCTCTCTAA) (SEQ ID NO: 106), miR-485-3p_FW15 (ATACACGGCTCTCCTCTCTAA) (SEQ ID NO: 107), or any combination thereof.

42. The method of claim 41, wherein the miR-485-3p primers comprise miR-485-3p_FW7.

43. The method of claim 41, wherein the miR-485-3p primers comprise miR-485-3p_FW2.

44. The method of claim 41, wherein the miR-485-3p primers comprise miR-485-3p_FW1.

45. The method of claim 41, wherein the miR-485-3p primers comprise miR-485-3p_FW9.

46. The method of any one of claims 32 to 45, further comprising administering a therapy capable of treating the cognitive disorder.

47. The method of any one of claims 11 to 31 and 46, wherein the therapy comprises a miR-485-3p inhibitor.

48. The method of claim 47, wherein the miR-485-3p inhibitor comprises a nucleotide sequence comprising 5′-UGUAUGA-3′ (SEQ ID NO: 2) and wherein the miR-485-3p inhibitor comprises about 6 to about 30 nucleotides in length.

49. The method of claim 47 or 48, wherein the miR-485-3p inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides at the ′5 of the nucleotide sequence; and/or wherein the miR-485-3p inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 3′ of the nucleotide sequence.

50. The method of any one of claims 47 to 49, wherein the miR-485-3p inhibitor comprises a nucleotide sequence selected from the group consisting of: 5′-UGUAUGA-3′ (SEQ ID NO: 2), 5′-GUGUAUGA-3′ (SEQ ID NO: 3), 5′-CGUGUAUGA-3′ (SEQ ID NO: 4), 5′-CCGUGUAUGA-3′ (SEQ ID NO: 5), 5′-GCCGUGUAUGA-3′ (SEQ ID NO: 6), 5′-AGCCGUGUAUGA-3′ (SEQ ID NO: 7), 5′-GAGCCGUGUAUGA-3′ (SEQ ID NO: 8), 5′-AGAGCCGUGUAUGA-3′ (SEQ ID NO: 9), 5′-GAGAGCCGUGUAUGA-3′ (SEQ ID NO: 10), 5′-GGAGAGCCGUGUAUGA-3′ (SEQ ID NO: 11), 5′-AGGAGAGCCGUGUAUGA-3′ (SEQ ID NO: 12), 5′-GAGGAGAGCCGUGUAUGA-3′ (SEQ ID NO: 13), 5′-AGAGGAGAGCCGUGUAUGA-3′ (SEQ ID NO: 14), 5′-GAGAGGAGAGCCGUGUAUGA-3′ (SEQ ID NO: 15); 5′-UGUAUGAC-3′ (SEQ ID NO: 16), 5′-GUGUAUGAC-3′ (SEQ ID NO: 17), 5′-CGUGUAUGAC-3′ (SEQ ID NO: 18), 5′-CCGUGUAUGAC-3′ (SEQ ID NO: 19), 5′-GCCGUGUAUGAC-3′ (SEQ ID NO: 20), 5′-AGCCGUGUAUGAC-3′ (SEQ ID NO: 21), 5′-GAGCCGUGUAUGAC-3′ (SEQ ID NO: 22), 5′-AGAGCCGUGUAUGAC-3′ (SEQ ID NO: 23), 5′-GAGAGCCGUGUAUGAC-3′ (SEQ ID NO: 24), 5′-GGAGAGCCGUGUAUGAC-3′ (SEQ ID NO: 25), 5′-AGGAGAGCCGUGUAUGAC-3′ (SEQ ID NO: 26), 5′-GAGGAGAGCCGUGUAUGAC-3′ (SEQ ID NO: 27), 5′-AGAGGAGAGCCGUGUAUGAC-3′ (SEQ ID NO: 28), 5′-GAGAGGAGAGCCGUGUAUGAC-3′ (SEQ ID NO: 29), and AGAGAGGAGAGCCGUGUAUGAC (SEQ ID NO: 30).

51. The method of any one of claims 47 to 49, wherein the miRNA inhibitor has a sequence selected from the group consisting of: 5′-TGTATGA-3′ (SEQ ID NO: 62), 5′-GTGTATGA-3′ (SEQ ID NO: 63), 5′-CGTGTATGA-3′ (SEQ ID NO: 64), 5′-CCGTGTATGA-3′ (SEQ ID NO: 65), 5′-GCCGTGTATGA-3′ (SEQ ID NO: 66), 5′-AGCCGTGTATGA-3′ (SEQ ID NO: 67), 5′-GAGCCGTGTATGA-3′ (SEQ ID NO: 68), 5′-AGAGCCGTGTATGA-3′ (SEQ ID NO: 69), 5′-GAGAGCCGTGTATGA-3′ (SEQ ID NO: 70), 5′-GGAGAGCCGTGTATGA-3′ (SEQ ID NO: 71), 5′-AGGAGAGCCGTGTATGA-3′ (SEQ ID NO: 72), 5′-GAGGAGAGCCGTGTATGA-3′ (SEQ ID NO: 73), 5′-AGAGGAGAGCCGTGTATGA-3′ (SEQ ID NO: 74), 5′-GAGAGGAGAGCCGTGTATGA-3′ (SEQ ID NO: 75); 5′-TGTATGAC-3′ (SEQ ID NO: 76), 5′-GTGTATGAC-3′ (SEQ ID NO: 77), 5′-CGTGTATGAC-3′ (SEQ ID NO: 78), 5′-CCGTGTATGAC-3′ (SEQ ID NO: 79), 5′-GCCGTGTATGAC-3′ (SEQ ID NO: 80), 5′-AGCCGTGTATGAC-3′ (SEQ ID NO: 81), 5′-GAGCCGTGTATGAC-3′ (SEQ ID NO: 82), 5′-AGAGCCGTGTATGAC-3′ (SEQ ID NO: 83), 5′-GAGAGCCGTGTATGAC-3′ (SEQ ID NO: 84), 5′-GGAGAGCCGTGTATGAC-3′ (SEQ ID NO: 85), 5′-AGGAGAGCCGTGTATGAC-3′ (SEQ ID NO: 86), 5′-GAGGAGAGCCGTGTATGAC-3′ (SEQ ID NO: 87), 5′-AGAGGAGAGCCGTGTATGAC-3′ (SEQ ID NO: 88), 5′-GAGAGGAGAGCCGTGTATGAC-3′ (SEQ ID NO: 89), and 5′-AGAGAGGAGAGCCGTGTATGAC-3′ (SEQ ID NO: 90).

52. The method of any one of claims 47 to 49, wherein the miR-485-3p inhibitor comprises a nucleotide sequence that is at least about 50%, at least about 55%, at least about 60%, at least about 65%, at least about 70%, at least about 75%, at least about 80%, at least about 85%, at least about 90%, or at least about 95% identical to 5′-AGAGAGGAGAGCCGUGUAUGAC-3′ (SEQ ID NO: 30) or 5′-AGAGAGGAGAGCCGTGTATGAC-3′ (SEQ ID NO: 90).

53. The method of claim 52, wherein the miR-485-3p inhibitor comprises a nucleotide sequence that is at least 90% identical to 5′-AGAGAGGAGAGCCGUGUAUGAC-3′ (SEQ ID NO: 30) or 5′-AGAGAGGAGAGCCGTGTATGAC-3′ (SEQ ID NO: 90).

54. The method of claim 52 or 53, wherein the miR-485-3p inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90) with one substitution or two substitutions.

55. The method of claim 52 or 53, wherein the miR-485-3p inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90).

56. The method of claim 55, wherein the miR-485-3p inhibitor comprises the nucleotide sequence 5′-AGAGAGGAGAGCCGUGUAUGAC-3′ (SEQ ID NO: 30).

57. The method of any one of claims 47 to 56, wherein the miR-485-3p inhibitor comprises at least one modified nucleotide.

58. The method of claim 57, wherein the at least one modified nucleotide comprises a locked nucleic acid (LNA), unlocked nucleic acid (UNA), arabino nucleic acid (ABA), bridged nucleic acid (BNA), peptide nucleic acid (PNA), or any combination thereof.

59. The method of any one of claims 47 to 57, wherein the miR-485-3p inhibitor comprises a backbone modification.

60. The method of claim 59, wherein the backbone modification comprises a phosphorodiamidate morpholino oligomer (PMO) and/or phosphorothioate (PS) modification.

61. The method of any one of claims 47 to 60, wherein the miR-485-3p inhibitor is delivered by a viral vector.

62. The method of claim 61, wherein the viral vector is an AAV, an adenovirus, a retrovirus, or a lentivirus.

63. The method of claim 62, wherein the viral vector is an AAV that has a serotype of AAV2, AAV3, AAV4, AAV5, AAV6, AAV7, AAV8, AAV9, AAV10, or any combination thereof.

64. The method of any one of claims 47 to 60, wherein the miR-485-3p inhibitor is delivered with a delivery agent.

65. The method of claim 64, wherein the delivery agent comprises a micelle, exosome, lipid nanoparticle, extracellular vesicle, synthetic vesicle, lipidoid, liposome, lipoplex, polymeric compound, peptide, protein, cell, nanoparticle mimic, nanotube, conjugate, or any combination thereof.

66. The method of claim 64 or 65, wherein the delivery agent comprises a cationic carrier unit comprising
[WP]-L1-[CC]-L2-[AM] (formula I)
or
[WP]-L1-[AM]-L2-[CC] (formula II)
wherein
WP is a water-soluble polymer moiety;
CC is a cationic carrier moiety;
AM is an adjuvant moiety; and,
L1 and L2 are independently optional linkers.

67. The method of claim 66, wherein the miRNA inhibitor and the cationic carrier unit are capable of associating with each other to form a micelle when mixed together.

68. The method of claim 67, wherein the association is via a covalent bond.

69. The method of claim 67, wherein the association is via a non-covalent bond.

70. The method of claim 69, wherein the non-covalent bond comprises an ionic bond.

71. The method of any one of claims 66 to 70, wherein the water-soluble polymer moiety comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof.

72. The method of any one of claims 66 to 71, wherein the water-soluble polymer moiety comprises polyethylene glycol (“PEG”), polyglycerol, or poly(propylene glycol) (“PPG”).

73. The method of any one of claims 66 to 72, wherein the water-soluble polymer moiety comprises: 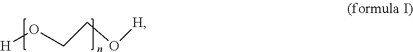
wherein n is 1-1000.

74. The method of claim 73, wherein the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141.

75. The method of claim 73, wherein the n is about 80 to about 90, about 90 to about 100, about 100 to about 110, about 110 to about 120, about 120 to about 130, about 140 to about 150, about 150 to about 160.

76. The method of any one of claims 66 to 75, wherein the water-soluble polymer moiety is linear, branched, or dendritic.

77. The method of any one of claims 66 to 76, wherein the cationic carrier moiety comprises one or more basic amino acids.

78. The method of claim 77, wherein the cationic carrier moiety comprises at least about three, at least about four, at least about five, at least about six, at least about seven, at least about eight, at least about nine, at least about ten, at least about 11, at least about 12, at least about 13, at least about 14, at last about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, or at least about 50 basic amino acids.

79. The method of claim 78, wherein the cationic carrier moiety comprises about 30 to about 50 basic amino acids.

80. The method of any one of claims 77 to 79, wherein the basic amino acid comprises arginine, lysine, histidine, or any combination thereof.

81. The method of any one of claims 77 to 80, wherein the cationic carrier moiety comprises about 40 lysine monomers.

82. The method of any one of claims 66 to 81, wherein the adjuvant moiety is capable of modulating an immune response, an inflammatory response, and/or a tissue microenvironment.

83. The method of any one of claims 66 to 82, wherein the adjuvant moiety comprises an imidazole derivative, an amino acid, a vitamin, or any combination thereof.

84. The method of claim 83, wherein the adjuvant moiety comprises: 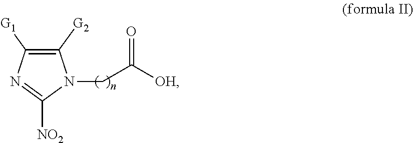
wherein each of G1 and G2 is H, an aromatic ring, or 1-10 alkyl, or G1 and G2 together form an aromatic ring, and wherein n is 1-10

85. The method of claim 83, wherein the adjuvant moiety comprises nitroimidazole.

86. The method of claim 83, wherein the adjuvant moiety comprises metronidazole, tinidazole, nimorazole, dimetridazole, pretomanid, ornidazole, megazol, azanidazole, benznidazole, or any combination thereof.

87. The method of any one of claims 66 to 83, wherein the adjuvant moiety comprises an amino acid.

88. The method of claim 87, wherein the adjuvant moiety comprises 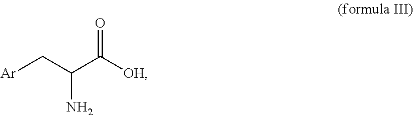
wherein Ar is 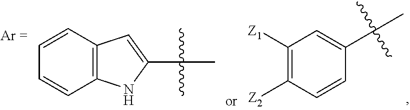
and
wherein each of Z1 and Z2 is H or OH.

89. The method of any one of claims 66 to 88, wherein the adjuvant moiety comprises a vitamin.

90. The method of claim 89, wherein the vitamin comprises a cyclic ring or cyclic hetero atom ring and a carboxyl group or hydroxyl group.

91. The method of claim 89 or 90, wherein the vitamin comprises: 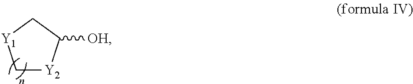
wherein each of Y1 and Y2 is C, N, O, or S, and wherein n is 1 or 2.

92. The method of any one of claims 89 to 91, wherein the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof.

93. The method of claim 92, wherein the vitamin is vitamin B3.

94. The method of claim 93, wherein the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3.

95. The method of claim 95, wherein the adjuvant moiety comprises about 10 vitamin B3.

96. The method of any one of claims 64 to 95, wherein the delivery agent comprises about a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

97. The method of any one of claims 64 to 96, wherein the delivery agent is associated with the miR-485-3p inhibitor, thereby forming a micelle.

98. The method of claim 97, wherein the association is a covalent bond, a non-covalent bond, or an ionic bond.

99. The method of claim 97 or 98, wherein the cationic carrier unit and the miR-485-3p inhibitor in the micelle is mixed in a solution so that the ionic ratio of the positive charges of the cationic carrier unit and the negative charges of the miR-485-3p inhibitor is about 1:1.

100. The method of any one of claims 66 to 99, wherein the cationic carrier unit is capable of protecting the miR-485-3p inhibitor from enzymatic degradation.

101. The method of any one of claims 1 to 100, wherein the cognitive disorder is associated with an increase in amyloid-beta accumulation within a region of the central nervous system (CNS) of the subject.

102. The method of claim 101, wherein the region of the CNS comprises a brain.

103. The method of any one of claims 1 to 102, wherein the cognitive disorder comprises an Alzheimer's Disease.

104. A composition comprising a miR-485-3p primer which comprises miR485-3p_FW1 (GTCATACACGGCTCTCCTCTCT) (SEQ ID NO: 94), miR485-3p_FW2 (TCATACACGGCTCTCCTCTC) (SEQ ID NO: 95), miR485-3p_FW3 (CATACACGGCTCTCCTCTC) (SEQ ID NO: 96), miR485-3p_FW4 (CATACACGGCTCTCCTCTCTA) (SEQ ID NO: 97), miR485-3p_FW5 (CATACACGGCTCTCGTCTC) (SEQ ID NO: 98), miR485-3p_FW6 (CATACACGGCTCTCGTCTCTAA) (SEQ ID NO: 99), miR485-3p_FW7 (GTCATACACGGCTCTCCTCTCTAA) (SEQ ID NO: 100), miR-485-3p_FW8 (GTCATACACGGCTCTCCTC) (SEQ ID NO: 101), miR-485-3p_FW9 (CATACACGGCTCTCCTCTCTAAA) (SEQ ID NO: 52), miR-485-3p_FW10 (GTCATACACGGCTCTCCTCTG) (SEQ ID NO: 102), miR-485-3p_FW11 (TCATACACGGCTCTCCTCTCT) (SEQ ID NO: 103), miR-485-3p_FW12 (TCATACACGGCTCTCCTC) (SEQ ID NO: 104), miR-485-3p_FW13 (TCATACACGGCTCTCCTCTCTAA) (SEQ ID NO: 105), miR-485-3p_FW14 (CATACACGGCTCTCCTCTCTAA) (SEQ ID NO: 106), miR-485-3p_FW15 (ATACACGGCTCTCCTCTCTAA) (SEQ ID NO: 107), or any combination thereof.

105. The composition of claim 104, wherein the miR-485-3p primer comprises miR-485-3p_FW7.

106. The composition of claim 104, wherein the miR-485-3p primer comprises miR-485-3p_FW2.

107. The composition of claim 104, wherein the miR-485-3p primer comprises miR-485-3p_FW1.

108. The composition of claim 104, wherein the miR-485-3p primer comprises miR-485-3p_FW9.
